(19) United States
(12) Patent Application Publication
(10) Pub. No.: US 2025/0319036 A1
(43) Pub. Date: Oct. 16, 2025

(54) BISPHOSPHONATE LIPIDS, LIPID NANOPARTICLE COMPOSITIONS COMPRISING THE SAME, AND METHODS OF USE THEREOF FOR TARGETED DELIVERY

(71) Applicant: The Trustees of the University of Pennsylvania, Philadelphia, PA (US)

(72) Inventors: Michael J. Mitchell, Philadelphia, PA (US); Lulu Xue, Philadelphia, PA (US); Drew Weissman, Philadelphia, PA (US); Mohamad-Gabriel Alameh, Philadelphia, PA (US)

(21) Appl. No.: 18/865,098

(22) PCT Filed: May 12, 2023
(86) PCT No.: PCT/US2023/066956

§ 371 Date: Nov. 12, 2024

Related U.S. Application Data
(60) Provisional application No. 63/341,753, filed on May 13, 2022.

(51) Int. Cl. A61K 9/51; A61K 31/7088; A61K 31/7105; A61K 31/711; A61K 38/46; A61K 47/10; A61K 47/24; A61K 47/28

(52) U.S. Cl. CPC A61K 9/5123; A61K 31/7088; A61K 31/7105; A61K 31/711; A61K 38/465; A61K 47/10; A61K 47/24; A61K 47/28

(57) ABSTRACT

Described herein, in some aspects, are bisphosphonate lipid compounds, lipid nanoparticles (LNPs) thereof, and methods of use thereof. In various embodiments, the LNP selectively targets a cell of interest (e.g., a bone cell and/or bone marrow cell, such as a stem cell, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasizes to bone, blood cancer cell, and multiple myeloma cell, inter alia). In other aspects, the present disclosure relates to methods for in vivo delivery of therapeutic agents to prevent or treat diseases, disorders, or conditions using the LNP compositions of the disclosure.

### CROSS-REFERENCE TO RELATED APPLICATIONS

This application claims priority under 35 U.S.C. § 119(e) to U.S. Provisional Patent Application No. 63/341,753, filed May 13, 2022, which is incorporated herein by reference in its entirety.

### STATEMENT REGARDING FEDERALLY SPONSORED RESEARCH OR DEVELOPMENT

This invention was made with government support under TR002776 awarded by the National Institutes of Health. The government has certain rights in the invention.

### BACKGROUND

The bone microenvironment is a distinct, highly dynamic region that comprises bone cells, cells of the hematopoietic and immune system, fibroblasts, stromal cells, and endothelial cells, as well as extracellular matrix (ECM) with abundant growth and/or signaling factors. These various cell types and ECM proteins enable orchestrated bone remodeling, hematopoiesis, immune function regulation, and tissue regeneration. Recently, increased incidences of various skeletal disease and age-related bone abnormalities, including osteoporosis, osteoarthritis, osteomyelitis and bone cancer, has inspired the investigation of novel biomaterials for bone microenvironment targeting.

However, various characteristics of the bone microenvironment, including reduced blood flow and vascularization compared to other organs, the blood-bone marrow barrier, poorly perfused bone sections, highly dense hierarchic structures, and low affinity between drugs and bone minerals, has generated several challenges for successful delivery of therapeutics into bone. Currently, organic/inorganic nanocomposites, and hydrogels have been developed for targeted and local delivery of pharmacological-based therapeutics, such as antibiotics, growth factors, anti-inflammatory molecules, anticancer agents, and hormones. However, low delivery efficiency of these materials necessitated high dosing to achieve ideal therapeutic dosage at diseased sites and long-term bioavailability, increasing the incidence of severe side effects. These limitations indicated the need for efficient and safe therapeutic approaches for targeted drug delivery to the bone microenvironment.

Thus, there is a need in the art for LNP delivery systems with high delivery efficacy and low toxicity to deliver RNA therapeutics, such as mRNA, gene therapy, gene editing technologies, etc., in a precise and efficient way to a bone cell and/or bone marrow cell. The present disclosure satisfies this unmet need.

### BRIEF SUMMARY

The present disclosure provides, in one aspect, bisphosphonate lipid compounds, lipid nanoparticles comprising the same, and methods of use thereof for targeted delivery.

In certain embodiments, the present disclosure provides a compound of Formula (I), or a or a racemate, enantiomer, diastereomer, pharmaceutically acceptable salt, solvate, or derivative thereof.

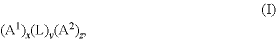

wherein each occurrence of A¹ is independently

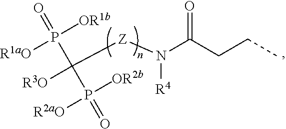

and each occurrence of A² is independently

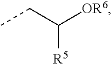

and each of L, R^{1a}, R^{1b}, R^{2a}, R^{2b}, R³, R⁴, R⁵, R⁶, Z, n, x, y, and z are defined elsewhere herein.

In certain embodiments, the present disclosure provide a lipid nanoparticle (LNP). In certain embodiments, the LNP comprises (a) at least one compound of Formula (I), or a racemate, enantiomer, diastereomer, pharmaceutically acceptable salt, solvate, or a derivative thereof. In certain embodiments, the LNP comprises (b) at least one neutral phospholipid, wherein the neutral phospholipid is present in a concentration range of about 5 mol % to about 45 mol %. In certain embodiments, the LNP comprises (c) at least one cholesterol lipid, wherein the total cholesterol lipid is in a concentration range of about 5 mol % to about 55 mol %. In certain embodiments, the LNP comprises (d) at least one polymer conjugated lipid (e.g., polyethylene glycol (PEG)-conjugated lipid). In certain embodiments, the total polymer conjugated lipid is present in a concentration range of about 0.5 mol % to about 12.5 mol %.

In certain embodiments, the present disclosure provides a composition comprising at least one LNP composition of the present disclosure.

In certain embodiments, the present disclosure provides a method of delivering an agent to a subject in need thereof. In certain embodiments, the method comprises administering a therapeutically effectively amount of at least one LNP of the present disclosure and/or a pharmaceutical composition of the present disclosure the same to the subject.

In certain embodiments, the present disclosure provides a method of delivering an agent to a bone in a subject, bone marrow, or a combination thereof. In certain embodiments, the method comprises administering a therapeutically effectively amount of at least one LNP of the present disclosure and/or a pharmaceutical composition of the present disclosure the same to the subject.

In certain embodiments, the present disclosure provides a method of treating, ameliorating, and/or preventing at least one disease, disorder, or condition in a subject in need thereof. In certain embodiments, the method comprises administering a therapeutically effectively amount of at least one LNP of the present disclosure and/or a pharmaceutical composition of the present disclosure the same to the subject.

In certain embodiments, the present disclosure provides a method of inducing a bone regeneration in a subject in need thereof. In certain embodiments, the method comprises administering a therapeutically effectively amount of at least one LNP of the present disclosure and/or a pharmaceutical composition of the present disclosure the same to the subject.

In certain embodiments, the present disclosure provides a method of replacing at least one protein in a subject in need thereof. In certain embodiments, the method comprises administering a therapeutically effectively amount of at least one LNP of the present disclosure and/or a pharmaceutical composition of the present disclosure the same to the subject.

In certain embodiments, the present disclosure provides a method of gene editing in a subject in need thereof. In certain embodiments, the method comprises administering a therapeutically effectively amount of at least one LNP of the present disclosure and/or a pharmaceutical composition of the present disclosure the same to the subject.

In certain embodiments, the present disclosure provides a method of inducing an immune response in a subject in need thereof. In certain embodiments, the method comprises administering a therapeutically effectively amount of at least one LNP of the present disclosure and/or a pharmaceutical composition of the present disclosure the same to the subject.

### BRIEF DESCRIPTION OF THE DRAWINGS

The following detailed description of illustrative embodiments of the disclosure will be better understood when read in conjunction with the appended drawings. For the purpose of illustrating the disclosure, illustrative embodiments are shown in the drawings. It should be understood, however, that the disclosure is not limited to the precise arrangements and instrumentalities of the embodiments shown in the drawings.

FIGS. 1A-ID depict a schematic representation of the rational design of BP lipid-like materials for mRNA delivery to the bone microenvironment in vivo. FIG. 1A depicts a schematic representation of lipid nanoparticles (LNPs) containing BP lipid-like materials (BP-LNPs) to enable systemic delivery of mRNA into the bone microenvironment. LNPs were prepared by combining four components (BP-lipid, DOPE, cholesterol and C14PEG2000) into each formulation using a microfluidic mixing device. After systemic delivery via intravenous injection, BP-LNPs coordinated with calcium ions (Ca²⁺) in the bone microenvironment to enable specific bone-targeting. FIG. 1B depicts representative results of ex vivo luminescence and fluorescence imaging of bones (left leg, spine, and right leg) after LNP delivery of mRNA encoding for luciferase and labelled with 1,1′-dioctadecyl-3,3,3′,3′-tetramethylindotricarbocyanine iodide (DiR) at a concentration of 10 μg/mL. Luminescence signal was generated where luciferase mRNA was translated, and fluorescence signal detected DiR signal from LNP biodistribution. FIG. 1C depicts representative cryogenic transmission electron microscopy (cryo-TEM) image of the morphology of BP-LNPs. Scale bar: 100 nm. FIG. 1D depicts representative results demonstrating hydrodynamic size distribution of representative BP-LNP. I.V., intravenous.

FIGS. 2A-2C, depicts a representative combinatorial library of BP lipid-like materials for mRNA delivery. Error bars represent SD. FIG. 2A depicts schematic representations of seven exemplary alendronate-conjugated polyamine cores (top) and three exemplary epoxide terminated alkyl tails (bottom) for generating twenty-one exemplary BP-lipids used in this study. BP-LNPs were named based on their BP-conjugated ionizable lipid component consisting of a BP-functionalized polyamine core (110BP, 197BP, T3ABP, 200BP, 488BP, 490BP and 494BP) and alkyl tail length (C12, C14 and C16). FIG. 2B depicts representative results demonstrating hydrodynamic size and PDI of the twenty-one exemplary formulated BP-LNPs. FIG. 2C depicts representative characterization of zeta potential and pKa for each BP-LNP formulation.

FIG. 3 depicts a schematic representation of the synthetic route of BP-lipids used in this study.

FIG. 4 depicts a representative proton nuclear magnetic resonance (¹H NMR) spectrum of N-acryloxysuccinimide (NAS).

FIG. 5 depicts a representative ¹H NMR spectrum of alendronate acrylamide.

FIG. 6 depicts a representative ¹H NMR spectrum of alendronate amide-bearing lipid core as exemplified by 110BP.

FIG. 7 depicts a representative ¹H NMR spectrum of alendronate amide-bearing lipid core T3ABP.

FIG. 8 depicts a representative ¹H NMR spectrum of alendronate amide-bearing lipid core 197BP.

FIG. 9 depicts a representative ¹H NMR spectrum of alendronate amide-bearing lipid core 200BP.

FIG. 10 depicts a representative ¹H NMR spectrum of alendronate amide-bearing lipid core 488BP.

FIG. 11 depicts a representative ¹H NMR spectrum of alendronate amide-bearing lipid core 490BP.

FIG. 12 depicts a representative ¹H NMR spectrum of alendronate amide-bearing lipid core 494BP.

FIGS. 13A-13B depict a schematic representation of 490BP-C14 and its corresponding ¹H NMR spectrum. FIG. 13A depicts a schematic representation of 490BP-C14. FIG. 13B depicts a representative ¹H NMR spectrum of 490BP-C14.

FIGS. 14A-14B depicts schematic representations of the lipid cores and tails used as a control in the library described herein. FIG. 14A depicts schematic representations of seven exemplary polyamine cores used in preparation of the library of twenty-one exemplary non-BP-lipids. FIG. 14B depicts schematic representations of three exemplary epoxide terminal alkyl tails used in preparation of the library of twenty-one exemplary non-BP-lipids.

FIG. 15 depicts representative results demonstrating mRNA encapsulation efficiency of twenty-one exemplary BP-LNPs. n=3 biological replicates. Error bars represent SEM.

FIGS. 16A-16G depict representative results demonstrating TNS fluorescence curves for BP-LNPs. The apparent pKa of an LNP was computed as the pH at which 50% of the TNS fluorescence was measured. FIG. 16A depicts representative results demonstrating TNS fluorescence curves for BNP-lipids derived from the 110BP-polyamine core. FIG. 16B depicts representative results demonstrating TNS fluorescence curves for BNP-lipids derived from the 197BP-polyamine core. FIG. 16C depicts representative results demonstrating TNS fluorescence curves for BNP-lipids derived from the T3ABP-polyamine core. FIG. 16D depicts representative results demonstrating TNS fluorescence curves for BNP-lipids derived from the 200BP-polyamine core. FIG. 16E depicts representative results demonstrating TNS fluorescence curves for BNP-lipids derived from the 484BP-polyamine core. FIG. 16F depicts representative results demonstrating TNS fluorescence curves for BNP-lipids derived from the 490BP-polyamine core. FIG. 16G depicts representative results demonstrating TNS fluorescence curves for BNP-lipids derived from the 494BP-polyamine core.

FIGS. 17A-17C depict representative results demonstrating in vitro screening of BP-LNPs and their binding capability to HA nanoparticles and bone fragments. Error bars in FIG. 17A and FIG. 17B represent SEM. FIG. 17A depicts representative results demonstrating luciferase expression in HeLa cells after treatment with the BP-LNP library. 5000 cells were incubated with LNPs at a dose of 10 ng/well. Results were normalized to untreated cells. n=3 biological replicates. FIG. 17B depicts representative results demonstrating quantitative evaluation of HA binding kinetics of 490BP-C14 LNPs and 490-C14 LNPs in the absence of BPs (single emulsion). FIG. 17C depicts representative fluorescence images of bone fragments before and after incubation with DiO solution (left), DiO labeled 490-C14 LNPs (center), and DiO labeled targeted 490BP-C14 LNPs (right). All samples were washed by PBS before imaging. Scale bar: 200 μm.

FIGS. 18A-18B depict representative results demonstrating in vitro screening and cell viability after 24 h transfection of LNPs in the absence of BPs of HeLa cells. 5000 cells were plated per well and treated with 10 ng mRNA. Error bars represent SEM. FIG. 18A depicts representative results demonstrating in vitro screening after 24 h transfection of LNPs in the absence of BPs of HeLa cells. FIG. 18B depicts representative results demonstrating in vitro cell viability after 24 h transfection of LNPs in the absence of BPs of HeLa cells.

FIG. 19 depicts representative results demonstrating cell viability after 24 h transfection of LNPs in the presence of BPs in HeLa cells. 5000 cells were plated per well and treated by 10 ng mRNA. Error bars represent SEM.

FIGS. 20A-20B depict representative results demonstrating in vitro and mRNA dependent transfection studies comparing 490BP-C14 LNP to 490-C14 LNP. mRNA dosages were 10 ng, 20 ng, 40 ng, 80 ng and 160 ng per well to treat cells. 5000 HeLa cells per well were used. Error bars represent SEM. FIG. 20A depicts representative results demonstrating in vitro transfection of HeLa cells by 490BP-C14 LNP and 490-C14 LNP at an mRNA dosage of 10 ng mRNA. FIG. 20B depicts representative results demonstrating mRNA dependent transfection of HeLa cells by 490BP-C14 LNP.

FIGS. 21A-21G depict representative results of in vivo delivery of mRNA and biodistribution of BP-LNPs to the bone microenvironment. Statistical significance in FIG. 21D and FIG. 21G was calculated using Student's t test with unpaired design. ****P<0.0001; ***P<0.001; **P<0.01. Error bars represent SEM. FIG. 21A depicts a schematic representation of DiR labeled LNPs delivering mRNA to the bone of mice. Bone fragments were further dissected for quantifying the transfection and homing efficiency of BP-LNPs. FIG. 21B depicts representative IVIS imaging of FLuc mRNA delivery to mice by 490-C14 and 490BP-C14 LNPs. Bone fragments show high luminescence intensity after 490BP-C14 LNP treatment. n=3 mice. FIG. 21C depicts representative left leg, spine and right leg dissected for luminescence imaging. FIG. 21D depicts representative results demonstrating the total luminescence quantification of dissected left leg, spine, and right leg for 490-C14 LNP and 490BP-C14 LNP treated mice. FIG. 21E depicts representative results demonstrating in vivo biodistribution of DiR labeled BP-LNPs. After introducing BP lipid-like materials into LNPs, the whole skeleton showed increased fluorescent signal. n=3 mice. FIG. 21F depicts representative results depicting the left leg, spine, and right leg that were dissected for fluorescence imaging. FIG. 21G depicts representative results demonstrating the total fluorescence radiance of dissected left leg, spine, and right leg for 490-C14 LNP and 490BP-C14 LNP treated mice.

FIGS. 22A-22D depict representative results demonstrating ex vivo luminescence and fluorescence intensity of organs in mice after delivering FLuc-mRNA LNPs labelled by DiR. n=3 mice H: heart; Li: liver; S: spleen; Lu: lung; K: Kidney. Error bars represent SEM. FIG. 22A depicts representative luminescence imaging of the main organs from mice with 490-C14 and 490BP-C14 LNPs treatment. FIG. 22B depicts representative results demonstrating luminescence quantification of organs from FIG. 22A. FIG. 22C depicts representative fluorescence imaging of the main organs from mice with 490-C14 and 490BP-C14 LNP treatment. FIG. 22D depicts representative results demonstrating fluorescence quantification of the organs from FIG. 22C.

FIG. 23 depicts representative results demonstrating time-dependence in vivo biodistribution of 490-C14 LNP and 490BP-C14 LNP.

FIGS. 24A-24D depict representative results demonstrating bone and bone marrow cell transfection and distribution following mRNA delivery using BP-LNPs. Statistical significance in FIG. 24B and FIG. 24D was calculated using Student's t test with unpaired design. ****P<0.0001; ***P<0.001; **P<0.01. Error bars represent SEM. FIG. 24A depicts representative results demonstrating gating for EGFP mRNA transfection in different cell types by 490-C14 and 490BP-C14 LNPs. T cells, monocytic lineage cells, B lineage cells, and monocytes were labelled by their corresponding markers. FIG. 24B depicts representative results demonstrating different EGFP⁺ cell populations following treatment of LNPs that incorporated (490BP-C14) or did not incorporate (490-C14) bone-targeting BPs. FIG. 24C depicts representative results demonstrating gating for DiR fluorescence in different cell types by 490-C14 and 490BP-C14 LNPs. T cells, monocytic lineage cells, B lineage cells, and monocytes were labelled by their corresponding markers. FIG. 24D depicts representative results demonstrating different DiR-loaded cell populations among DiR cells. n=3 mice.

FIGS. 25A-25E depict representative results demonstrating EGFP mRNA transfects diverse types of cells in the bone marrow. Experiments were performed for three or more replicates. Error bars represent SEM. FIG. 25A depicts a representative results demonstrating gating strategy to identify cell types in bone marrow. FIG. 25B depicts representative results demonstrating EGFP mRNA transfection in B cells, endothelial cells, and granulocytes by 490-C14 LNPs and 490BP-C14 LNPs. FIG. 25C depicts representative results demonstrating EGFP⁺ populations of LNPs treated B cells, endothelial cells, granulocytes, and HSC cells. FIG. 25D depicts representative results demonstrating DiR signal in B cells, endothelial cells, and granulocytes after administration of 490-C14 LNPs and 490BP-C14 LNPs. FIG. 25E depicts representative results demonstrating DiR⁺ populations of LNP treated B cells, endothelial cells, granulocytes, and HSC cells.

FIGS. 26A-26B depict representative results demonstrating EGFP transfection and fluorescent biodistribution of 490BP-C14 LNPs relative to 490-C14 LNPs in various cell types. FIG. 26A depicts representative results demonstrating EGFP transfection in various cell types in bone marrow treated with 490BP-C14 LNPs, represented as fold of increase in the EGFP⁺ cell population treated with 490BP-C14 LNPs compared to those treated with 490-C14 LNPs. FIG. 26B depicts representative results demonstrating fluorescent biodistribution in various cell types in bone marrow treated with 490BP-C14 LNPs, represented as fold of increase in the DiR+ cell population treated with 490BP-C14 LNPs compared to 490-C14 LNPs.

FIGS. 27A-27B depict representative result demonstrating BMP-2 protein secretion in the bone microenvironment following delivery of BP-LNPs encapsulating mRNA encoding for BMP-2 via intravenous injection. Statistical significance in FIG. 27A and FIG. 27B was calculated using Student's t test with unpaired design. **P<0.01; *P<0.05. Error bars represent SEM. FIG. 27A depicts representative results demonstrating BMP-2 secretion from the bone surface following BP-LNP mRNA delivery. BMP-2 was extracted from the bone surface by incubating bone samples with 4M guanidine hydrochloride overnight. FIG. 27B depicts representative results demonstrating BMP-2 secretion in the bone marrow with increased BMP-2 mRNA dosing. BMP-2 was extruded out from the bone marrow. n=3 mice.

FIGS. 28A-28B depict representative results demonstrating liver toxicity assay after injection of LNPs encapsulating luciferase-encoding mRNA. C57BL/6J mice were dosed with 1.5 mg/kg luciferase mRNA LNPs, and liver enzymes were quantified 12 h after injection. Error bars represent SEM. FIG. 28A depicts representative results demonstrating alanine transaminase (ALT) quantification (±standard deviation) for controls, 490-C14 LNPs and 490BP-C14 LNPs. n=3 mice. FIG. 28B depicts representative results demonstrating aspartate transaminase (AST) quantification (±standard deviation) for controls, 490-C14 LNPs and 490BP-C14 LNPs. n=3 mice.

FIG. 29 provides representative images of EGFP expression in the femur after administration of 490BP-C14 LNPs encapsulating EGFP mRNA to the bone microenvironment by intravenous administration. The bone femur was dissected 12 h post injection. DAPI was used for nuclear staining. The mRNA dosage was 0.5 mg/kg. EGFP expression was detected in the bone marrow, especially in the endosteum, rather than on the bone surface.

FIGS. 30A-30C: representative images of BMP-2 expression in the tibia bones of mice. FIG. 30A: tibia bone from PBS treated mice. FIG. 30B: tibia bone was dissected from mice treated with 490-C14 LNPs delivering BMP-2 mRNA. FIG. 30C: tibia bond was dissected from mice treated with 490BP-C14 LNPs delivering BMP-2 mRNA. The magnified regions represent BMP-2 expression. Scale bar: 500 μm. DAPI (blue) was used for nuclear staining, and BMP2 expression is shown in green.

FIGS. 31A-31C: stability of 490BP-C14 LNPs after storage at 4° C. for 21 days. FIG. 31A: particle size quantification at different time points. FIG. 31B: mRNA encapsulation efficiency at different time points. FIG. 31C: luciferase expression in HeLa cells after treatment with 490BP-C14 LNPs at different time points. 5000 cells were incubated with LNPs at a dose of 10 ng/well. Results were normalized to untreated cells; n=3 replicates.

FIGS. 32A-32B: ex vivo imaging of bones after Fluc mRNA delivery to mice by 490-C14 LNPs and 490BP-C14 LNPs (Fluc mRNA dose: 0.25 mg/kg). Fore limbs, hind limbs, and spine were dissected for imaging. Both the luminescence signal and fluorescent signal increased in 490BP-LNP treated group compared to the 490-C14 LNP control. FIG. 32A: luminescent intensity of the bone sites. FIG. 32B: fluorescence intensity of the bone sites. DiR fluorescent dye was used to label the LNPs.

FIGS. 33A-33C: representative H&E staining of spine from PBS (FIG. 33A), 490-C14 LNP (FIG. 33B), and 490BP-C14 LNP (FIG. 33C).

FIG. 34A: EGFR transfection in various cell types in bone marrow treated with 490BP-C14 LNPs, represented as fold of increase in the EGFP⁺ cell population treated with 490BP-C14 LNPs compared to those treated with 490-C14 LNPs. FIG. 34B: fluorescent biodistribution in various cell types in bone marrow treated with 490BP-C14 LNPs, represented as fold of increase in the DiR+ cell population treated with 490BP-C14 LNPs compared to 490-C14 LNPs.

FIGS. 35A-35B: representative immunofluorescence of Smad1/5 activation in the bone microenvironment of mouse tibia before and after BMP-2 mRNA delivery by LNPs. FIG. 35A: Smad1/5 activation in metaphyseal region of mice tibia. FIG. 35B: Smad1/5 activation in endosteal region of mice tibia. Scale bar: 200 μm. DAPI (blue) was used for nuclear staining and phosphorylated Smad1/5 was shown in green.

FIGS. 36A-36B: H&H staining of representative bone sites of PBS, 490-C14 LNP, and 490BP-C14 LNP treated mice. FIG. 36A: representative images of cortical bone and endosteal bone marrow of femur bones. FIG. 36B: representative images of trabecular bone and metaphysis bone marrow of femur bones. Scale bar: 100 μm.

### DETAILED DESCRIPTION

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y. or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” as used herein when referring to a measurable value, for example numerical values and/or ranges, such as an amount, a temporal duration, and the like, is meant to encompass variations of ±20%, ±10%, ±5%, ±1%, or ±0.1% from the specified value, as such variations are appropriate to perform the disclosed methods. For example, “about 40 [units]” may mean within +25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein.

The term “abnormal” when used in the context of organisms, tissues, cells or components thereof, refers to those organisms, tissues, cells or components thereof that differ in at least one observable or detectable characteristic (e.g., age, treatment, time of day, and so forth) from those organisms, tissues, cells or components thereof that display the “normal” (expected) respective characteristic. Characteristics which are normal or expected for one cell or tissue type, might be abnormal for a different cell or tissue type.

The term “adjuvant” as used herein is defined as any molecule to enhance an antigen-specific adaptive immune response.

A disease or disorder is “alleviated” if the severity of a sign or symptom of the disease or disorder, the frequency with which such a sign or symptom is experienced by a patient, or both, is reduced.

“Alkenyl” or “alkenyl group” refers to a straight or branched hydrocarbon chain having from 2 to 20 carbon atoms, and having one or more carbon-carbon double bonds. Each alkenyl group is attached to the rest of the molecule by a single bond. Alkenyl group comprising any number of carbon atoms from 2 to 20 are included. In some embodiments, the alkenyl is a C₂-C₂₀ alkenyl, a C₂-C₁₂ alkenyl, a C₂-C₁₀ alkenyl, a C₂-C₈ alkenyl, a C₂-C₆ alkenyl, a C₂-C₄ alkenyl, or a C₂-C₃ alkenyl. An alkenyl group comprising up to 20 carbon atoms is a C₂-C₂₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₂₀ alkenyl includes all the foregoing moieties, but also includes C₁₁, C₁₂, C₁₃, C₁₄, C₁₅, C₁₆, C₁₇, C₁₈, C₁₉, and C₂₀ alkenyls. Non-limiting examples of C₂-C₂₀ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

As used herein, the term “alkoxy” employed alone or in combination with other terms means, unless otherwise stated, refers to a group of the formula —ORₐ where Rₐ is an alkyl, alkenyl or alknyl group having from 1 to 20 carbon atoms, as defined above, connected to the rest of the molecule via an oxygen atom, such as, for example, methoxy, ethoxy, 1-propoxy, 2-propoxy (isopropoxy) and the higher homologs and isomers. Unless stated otherwise specifically in the specification, an alkoxy group can be optionally substituted.

As used herein, the term “alkyl,” or “alkyl group” by itself or as part of another substituent means, unless otherwise stated, a straight or branched chain hydrocarbon having from 1 to 20 carbon atoms. In some embodiments, the alkyl is a C₁-C₂₀ alkyl, a C₁-C₁₂ alkyl, a C₁-C₁₀ alkyl, a C₁-C₈ alkyl, a C₁-C₆ alkyl, a C₁-C₄ alkyl, or a C₁-C₃ alkyl. For example, an alkyl comprising up to 20 carbon atoms is a C₁-C₂₀ alkyl, an alkyl comprising up to 12 carbon atoms is a C₁-C₁₂ alkyl, an alkyl comprising up to 10 carbon atoms is a C₁-C₁₀ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₂₀ alkyl includes all the foregoing moieties, but also includes C₁₁, C₁₂, C₁₃, C₁₄, C₁₅, C₁₆, C₁₇, C₁₈, C₁₉, and C₂₀ alkyls. Non-limiting examples include methyl, ethyl, n-propyl, isopropyl, n-butyl, isobutyl, sec-butyl, tert-butyl, n-pentyl, neopentyl, n-hexyl, n-heptyl, n-octyl, n-nonyl, n-decyl, n-undecyl, n-dodecyl, and cyclopropylmethyl. Unless stated otherwise specifically in the specification, an alkyl group can be optionally substituted.

“Alkylamino” refers to a group of the formula —NHRₐ or —NRₐRₐ where each Rₐ is, independently, an alkyl, alkenyl or alkynyl group as defined above containing 1 to 20 carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl group as defined above. A non-limiting example of an alkyl carbonyl is the methyl carbonyl (“acetal”) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₁-C₁₀ alkenyl, or C₁-C₁₀ alkynyl group as defined above. Unless stated otherwise specifically in the specification, an alkyl carbonyl group can be optionally substituted.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms.

“Alkynyl” or “alkynyl group” refers to a straight or branched hydrocarbon chain having from 2 to 20 carbon atoms, and having one or more carbon-carbon triple bonds. Each alkynyl group is attached to the rest of the molecule by a single bond. In some embodiments, the alkynyl is a C₂-C₂₀ alkynyl, a C₂-C₁₂ alkynyl, a C₂-C₁₀ alkynyl, a C₂-C₈ alkynyl, a C₂-C₆ alkynyl, a C₂-C₄ alkynyl, or a C₂-C₃ alkynyl. Alkynyl group comprising any number of carbon atoms from 2 to 20 are included. An alkynyl group comprising up to 20 carbon atoms is a C₂-C₂₀ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₂₀ alkynyl includes all the foregoing moieties, but also includes C₁₁, C₁₂, C₁₃, C₁₄, C₁₅, C₁₆, C₁₇, C₁₈, C₁₉, and C₂₀ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

The term “amino” refers to a group of the formula —NRₐRₐ, —NHRₐ, or —NH₂, where each Rₐ is, independently, an alkyl (e.g., aminoalkyl), alkenyl or alkynyl group as defined above containing 1 to 20 carbon atoms. Unless stated otherwise specifically in the specification, an alkylamino or aminoalkyl group can be optionally substituted.

As used herein, the terms “amino acid”, “amino acidic monomer”, or “amino acid residue” refer to any of the twenty naturally occurring amino acids including synthetic amino acids with unnatural side chains and including both D and L optical isomers.

The “aminoalkyl linker” or “aminoalkylenyl” as used herein refers to a bivalent, at least partially saturated, aliphatic diradical comprising at least one nitrogen atom. In certain embodiments, the nitrogen atom has a lone pair. In certain embodiments, the term may be regarded as a moiety derived from the corresponding aminoalkyl by removal of two hydrogen atoms from the same or different carbon atom and/or heteroatom(s). The terms “mono”, “di”, “tri”, “tetra”, “penta”, and the like, used in conjunction with the term “aminoalkyl linker” indicate the number of nitrogen atoms comprising the moiety (i.e., a triaminoalkyl linker comprises three nitrogen atoms).

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, prodrug, or other variant of the reference molecule.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present disclosure may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments. linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. k and l light chains refer to the two major antibody light chain isotypes.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The terms “aralkenyl” or “arylalkenyl” refer to a radical of the formula —R_{b}—R_{c} where R_{b} is an alkenylene o group as defined above and R_{c} is one or more aryl radicals as defined above. Unless stated otherwise specifically in the specification, an aralkenyl group can be optionally substituted.

The terms “aralkyl” or “arylalkyl” refer to a radical of the formula —R_{b}—R_{c} where R_{b} is an alkylene group as defined above and R_{c} is one or more aryl radicals as defined above, for example, benzyl, diphenylmethyl and the like. Unless stated otherwise specifically in the specification, an aralkyl group can be optionally substituted.

The terms “aralkynyl” or “arylalkynyl” refer to a radical of the formula —R_{b}—R_{c} where R_{b} is an alkynylene group as defined above and R_{c} is one or more aryl radicals as defined above. Unless stated otherwise specifically in the specification, an aralkynyl group can be optionally substituted.

As used herein, the term “aromatic” refers to a carbocyclyl or heterocyclyl with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a hydrocarbon ring system, comprising hydrogen, 6 to 18 carbon atoms and at least one aromatic ring. For purposes of this disclosure, the aryl can be a monocyclic, bicyclic, tricyclic or tetracyclic ring system, which can include fused or bridged ring systems. For example, aryls include, but are not limited to, a biphenyl, or may be fused, such as naphthalene. Examples of aryl groups include benzyl, indacenyl, pyrenyl, triphenyl, phenyl, anthracyl, and naphthyl. Unless stated otherwise specifically in the specification, the term “aryl” is meant to include aryl groups that are optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl, cycloalkenyl and cycloalkynyl as defined herein. Unless stated otherwise specifically in the specification, a carbocyclyl group can be optionally substituted.

The term “compound,” as used herein, unless otherwise indicated, refers to any specific chemical compound disclosed herein. In certain embodiments, the term also refers to stereoisomers and/or optical isomers (including racemic mixtures) or enantiomerically enriched mixtures of disclosed compounds.

The term “cyano” refers to a group of the formula —CN group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

Monocyclic cycloalkyls include, but are not limited to, cyclopropyl, cyclobutyl, cyclopentyl, cyclohexyl, cycloheptyl, and cyclooctyl. Dicyclic or polycyclic cycloalkyls include, but are not limited to, tetrahydronaphthyl, indanyl, and tetrahydropentalenyl, adamantyl and norbornyl. The term cycloalkyl includes “unsaturated nonaromatic carbocyclyl,” “carbocyclyl,” “carbocyclic ring,” “carbocycle,” or “nonaromatic unsaturated carbocyclyl” groups, both of which refer to a nonaromatic carbocycle as defined herein, which contains at least one carbon double bond or one carbon triple bond.

The term “cycloalkylene” or “cycloalkylenyl” as used herein refers to a bivalent saturated cycloalkyl radical

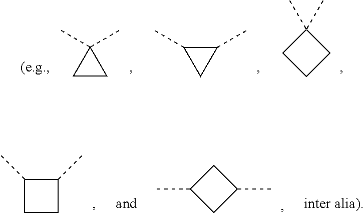

In certain embodiments, the term may be regarded as a product of removal of two hydrogen atoms from the corresponding cycloalkane (e.g., cyclobutyl) by removal of two hydrogen atoms from the same

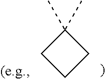

different

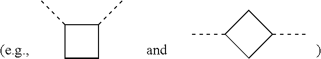

carbon atoms.

“Cycloalkenyl” refers to a stable non aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyls include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” or “therapeutically effective amount”, as used herein, means an amount which provides a therapeutic or prophylactic benefit. An “effective amount” or “therapeutically effective amount” of a compound is that amount of compound which is sufficient to provide a beneficial effect to the subject to which the compound is administered. For example, a “therapeutically effective amount” of the LNPs is the amount that is sufficient or effective to prevent or treat (delay or prevent the onset of, prevent the progression of, inhibit, decrease or reverse) a disease or condition, including alleviating symptoms of such diseases.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “halo” or “halogen” alone or as part of another substituent means, unless otherwise stated, a fluorine, chlorine, bromine, or iodine group.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

As used herein, the term “heteroalkyl” by itself or in combination with another term means, unless otherwise stated, a stable straight or branched chain alkyl group consisting of from 1 to 20 carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom can be optionally substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The term heterocycloalkyl group can also be a C₂ heterocycloalkyl, C₂-C₃ heterocycloalkyl, C₂-C₄ heterocycloalkyl, C₂-C₅ heterocycloalkyl, C₂-C₆ heterocycloalkyl, C₂-C₇ heterocycloalkyl, C₂-C₈ heterocycloalkyl, C₂-C₉ heterocycloalkyl, C₂-C₁₀ heterocycloalkyl, C₂-C₁₁ heterocycloalkyl, and the like, up to and including a C₂-14₅ heterocycloalkyl. For example, a C₂ heterocycloalkyl comprises a group which has two carbon atoms and at least one heteroatom, including, but not limited to, aziridinyl, diazetidinyl, oxiranyl, thiiranyl, and the like. Alternatively, for example, a C₅ heterocycloalkyl comprises a group which has five carbon atoms and at least one heteroatom, including, but not limited to, piperidinyl, tetrahydropyranyl, tetrahydrothiopyranyl, diazepanyl, and the like. It is understood that a heterocycloalkyl group may be bound either through a heteroatom in the ring, where chemically possible, or one of carbons comprising the heterocycloalkyl ring. The heterocycloalkyl group can be substituted or unsubstituted.

The term “heterocycloalkylene” or “heterocycloalkylenyl” as used herein refers to a bivalent saturated cycloalkyl radical

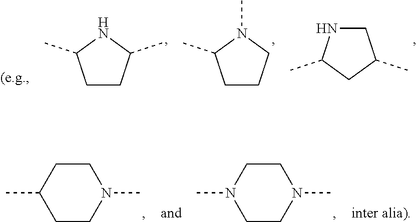

In certain embodiments, the term may be regarded as a product of removal of two hydrogen atoms from the corresponding heterocycloalkane (e.g., piperidine) by removal of two hydrogen atoms from the same

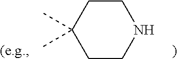

different

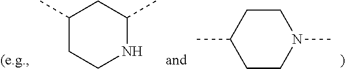

carbon atom(s) and/or heteroatom(s).

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl. oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylene” or “heteroarylenyl” as used herein refers to a bivalent heteroaryl radical (e.g., 2,4-pyridylene). In certain embodiments, the term may be regarded as a divalent radical formed by the removal of two hydrogen atoms from one or more rings of a heteroaryl moiety, wherein the hydrogen atoms may be removed from the same or different rings, preferably the same ring.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}—R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}—R_{f} where R_{b} is an alkynylene chain as defined above and R_{f} is a heteroaryl radical as defined above. Unless stated otherwise specifically in the specification, a heteroarylalkynyl group can be optionally substituted.

As used herein, the term “substituted” means any of the above groups (i.e., alkyl, alkylene, alkenyl, alkynyl, alkoxy, aryl, carbocyclyl, cycloalkyl, cycloalkenyl, cycloalkynyl, heterocyclyl, and/or heteroaryl) wherein at least hydrogen atom is replaced by a bond to a non-hydrogen atom or group of atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. The term “substituted” further refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In yet other embodiments, the substituents vary in number between one and three. In certain embodiments, the substituents vary in number between one and two. “Substituted” also means any of the above groups in which one or more hydrogen atoms are replaced by a higher-order bond (e.g., a double- or triple-bond) to a heteroatom such as oxygen in oxo, carbonyl, carboxyl, carboxylic acid and ester groups; and nitrogen in groups such as imines, oximes, hydrazones, and nitriles. For example, “substituted” includes any of the above groups in which one or more hydrogen atoms are replaced with, for example, —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with, for example, —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently selected from any of the above groups, including but not limited to: hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to any of the above groups, including but not limited to amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group.

In addition, each of the foregoing substituents can also be optionally substituted with one or more of the above substituents.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “hydroxy” or “hydroxyl” refers to a group of the formula —OH group.

“Immunogen” refers to any substance introduced into the body in order to generate an immune response. That substance can a physical molecule, such as a protein, or can be encoded by a vector, such as DNA, mRNA, or a virus.

The term “imino” refers to a group of the formula ═NH group.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “linker” as used herein refers to an organic moiety that connects two parts of a compound (e.g., a small molecule drug and an antibody). The linker can be, in non-limiting examples, a direct bond, a single atom (e.g., —O—), a peptide, or a substituted or unsubstituted alkylene or heteroalkylene moiety (e.g., polyethylene glycol). One skilled in the art would be apprised of the common linkers suitable for use in antibody drug conjugates and methods of preparation thereof.

The term “isomers” or “stereoisomers” refers to compounds, which have identical chemical constitution, but differ with regard to the arrangement of the atoms or groups in space.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The term “nitro” refers to a group of the formula —NO₂ group.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

As used herein, the term “optionally substituted” means that the referenced group may be substituted or unsubstituted. In certain embodiments, the referenced group is optionally substituted with zero substituents, i.e., the referenced group is unsubstituted. In yet other embodiments, the referenced group is optionally substituted with one or more additional group(s) individually and independently selected from groups described herein.

The term “oxo” refers to a group of the formula the ═O group.

“Parenteral” administration of a composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “pharmacological composition,” “therapeutic composition,” “therapeutic formulation” or “pharmaceutically acceptable formulation” can mean, but is in no way limited to, a composition or formulation that allows for the effective distribution of an agent provided by the disclosure, which is in a form suitable for administration to the physical location most suitable for their desired activity, e.g., systemic administration. The pharmaceutical composition facilitates administration of the compound to an organism. Multiple techniques of administering a compound exist in the art including, but not limited to, intravenous, oral, aerosol, parenteral, ophthalmic, pulmonary and topical administration.

Non-limiting examples of agents suitable for formulation with the, e.g., compounds provided by the instant disclosure include: cinnamoyl, PEG, phospholipids or lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the disclosure is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

As used herein, the term “prodrug” refers to an agent that is converted into the parent drug in vivo. For example, the term “prodrug” refers to a derivative of a known direct acting drug, which derivative has enhanced delivery characteristics and therapeutic value as compared to the drug, and is transformed into the active drug by an enzymatic or chemical process. In some embodiments, “prodrug” refers to an inactive or relatively less active form of an active agent that becomes active by undergoing a chemical conversion through one or more metabolic processes. In certain embodiments, upon in vivo administration, a prodrug is chemically converted to the biologically, pharmaceutically, or therapeutically active form of the compound. In yet other embodiments, a prodrug is enzymatically metabolized by one or more steps or processes to the biologically, pharmaceutically, or therapeutically active form of the compound. For example, the present compounds can be administered to a subject as a prodrug that includes an initiator bound to an active agent, and, by virtue of being degraded by a metabolic process, the active agent is released in its active form.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

In certain embodiments, “pseudouridine” refers, in yet other embodiments, to m¹acp³Y (1-methyl-3-(3-amino-3-carboxypropyl) pseudouridine. In yet other embodiments, the term refers to m¹Y (1-methylpseudouridine). In yet other embodiments, the term refers to Ym (2′-O-methylpseudouridine. In yet other embodiments, the term refers to m⁵D (5-methyldihydrouridine). In yet other embodiments, the term refers to m³Y (3-methylpseudouridine). In yet other embodiments, the term refers to a pseudouridine moiety that is not further modified. In yet other embodiments, the term refers to a monophosphate, diphosphate, or triphosphate of any of the above pseudouridines. In yet other embodiments, the term refers to any other pseudouridine known in the art. Each possibility represents a separate embodiment of the present disclosure.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “tautomers” are constitutional isomers of organic compounds that readily interconvert by a chemical process (tautomerization).

The term “therapeutic” as used herein means a treatment and/or prophylaxis. A therapeutic effect is obtained by suppression, diminution, remission, or eradication of at least one sign or symptom of a disease or disorder state.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, hydrophilic therapeutic agents, hydrophobic therapeutic agents, antibiotics, antibodies, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

Some examples of active ingredients suitable for use in the pharmaceutical formulations and methods of the present disclosure include: hydrophilic, lipophilic, amphiphilic or hydrophobic, and that can be solubilized, dispersed, or partially solubilized and dispersed, on or about the nanocluster. The active agent-nanocluster combination may be coated further to encapsulate the agent-nanocluster combination and may be directed to a target by functionalizing the nanocluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs and derivatives.

“Thioalkyl” refers to a formula —SRₐ where Rₐ is an alkyl, alkenyl, or alkynyl as defined above containing 1 to 20 carbon atoms. Unless stated otherwise specifically in the specification, a thioalkyl group can be optionally substituted.

The term “thioxo” refers to a group of the formula the ═S group.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

As used herein, “treating a disease or disorder” means reducing the frequency with which a symptom of the disease or disorder is experienced by a patient. Disease and disorder are used interchangeably herein.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, and so forth, as well as individual numbers within that range, for example, 1, 2, 2.7, 3, 4, 5, 5.3, and 6. This applies regardless of the breadth of the range.

### Bisphosphonate Lipid Compounds

In one aspect, the present disclosure provides a compound having the structure of Formula (I), or a racemate, enantiomer, diastereomer, pharmaceutically acceptable salt, solvate, or derivative thereof:

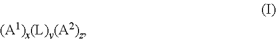

wherein:
each occurrence of A¹ is independently

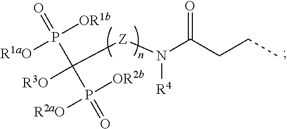

each occurrence of A² is independently

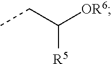

each occurrence of L is an amine linker independently selected from the group consisting of aminoalkyl linker, substituted aminoalkyl linker, diaminoalkyl linker, substituted diaminoalkyl linker, triaminoalkyl linker, substituted triaminoalkyl linker, tetraaminoalkyl linker, substituted tetraaminoalkyl linker, pentaaminoalkyl linker, substituted pentaaminoalkyl linker, poly aminoalkyl linker, substituted poly aminoalkyl linker, aminocycloalkyl linker, substituted aminocycloalkyl linker, diaminocycloalkyl linker, substituted diaminocycloalkyl linker, triaminocycloalkyl linker, substituted triaminocycloalkyl linker, tetraaminocycloalkyl linker, substituted tetraaminocycloalkyl linker, pentaaminocycloalkyl linker, substituted pentaaminocycloalkyl linker, polyaminocycloalkyl linker, substituted poly aminocycloalkyl linker, and any combination thereof;
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si;
each occurrence of z′ and z″ is independently an integer represented by 0, 1, or 2;
wherein x, y, and z are independently an integer from 0 to 20;
each occurrence of n is independently an integer from 0 to 10.

In certain embodiments, L is selected from the group consisting of

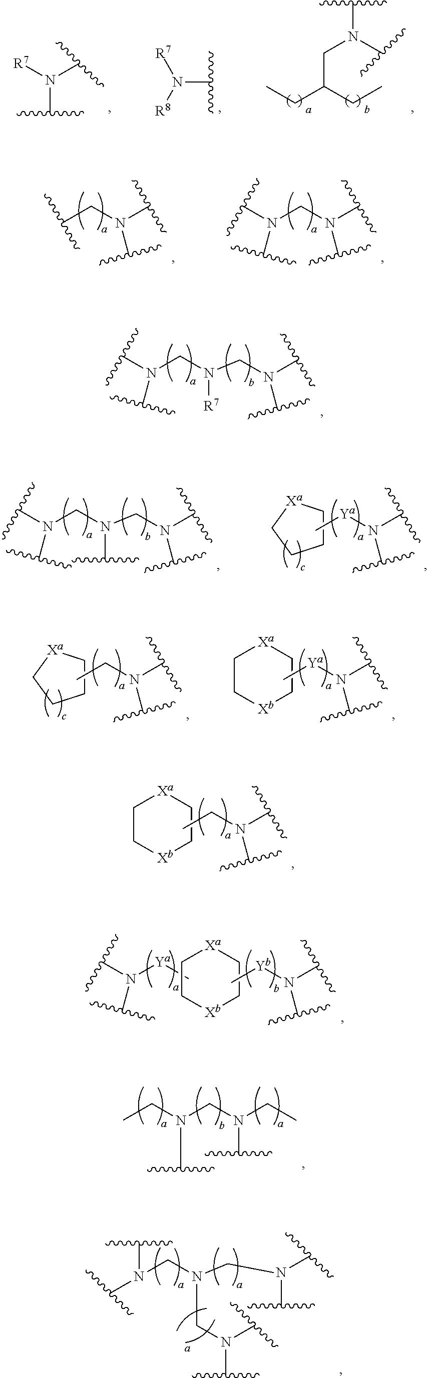

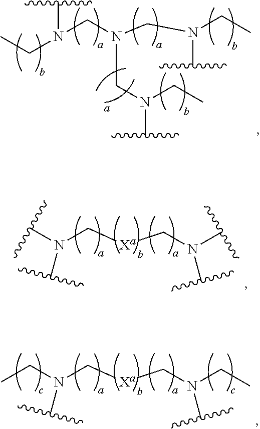

and any combination thereof,
wherein:
each occurrence of R⁷ and R⁸ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of X^{a} and X^{b} is independently selected from the group consisting of O, S, N(R⁹)_{z′}, P(R⁹)_{z′}, and any combination thereof;
each occurrence of Y^{a} and Y^{b} is independently selected from the group consisting of C₁-C₁₂ alkylenyl, substituted C₁-C₁₂ alkylenyl, C₃-C₈ cycloalkylenyl, substituted C₃-C₈ cycloalkylenyl, —Y′(R⁹)_{z′}(R¹⁰)_{z″}—(C₃-C₈ cycloalkylenyl), substituted —Y′(R⁹)_{z′}(R¹⁰)_{z″}—C₃-C₈ cycloalkylenyl, C₂-C₈ heterocycloalkylenyl, substituted C₂-C₈ heterocycloalkylenyl, —Y′(R⁹)_{z′}(R¹⁰)_{z″}—C₂-C₈ heterocycloalkylenyl, substituted-Y′(R⁹)_{z′}(R¹⁰)_{z″}—C₂-C₈ heterocycloalkylenyl, C₂-C₈ alkenylenyl, substituted C₂-C₈ alkenylenyl, C₅-C₁₀ cycloalkenylenyl, substituted C₅-C₁₀ cycloalkenylenyl, —Y′(R⁹)_{z′}(R¹⁰)_{z″}—C₅-C₁₀ cycloalkenylenyl, substituted —Y′(R⁹)_{z′}(R¹⁰)_{z″}—C₅-C₁₀ cycloalkenylenyl, C₂-C₈ alkynylenyl, substituted C₂-C₈ alkynylenyl, C₅-C₁₂ cycloalkynylenyl, substituted C₅-C₁₂ cycloalkynylenyl, —Y′(R⁹)_{z′}(R¹⁰)_{z″}—C₈-C₁₂ cycloalkynylenyl, substituted —Y′(R⁹)_{z′}(R¹⁰)_{z″}—C₈-C₁₂ cycloalkynylenyl, C₆-C₁₀ arylenyl. substituted C₆-C₁₀ arylenyl, —Y′(R⁹)_{z′}(R¹⁰)_{z″}—C₆-C₁₀ arylenyl, substituted —Y′(R⁹)_{z′}(R¹⁰)_{z″}—C₆-C₁₀ arylenyl, C₂-C₁₀ heteroarylenyl, substituted C₂-C₁₀ heteroarylenyl, —Y′(R⁹)_{z′}(R¹⁰)_{z″}—C₂-C₁₀ heteroarylenyl, and substituted —Y′(R⁹)_{z′}(R¹⁰)_{z″}—C₂-C₁₀ heteroarylenyl;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2;
wherein each occurrence of a, b, and c is independently an integer from 0 to 10;
each occurrence of

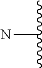 [SMILES: *N]

indicates a bond between a N atom of L and A¹ or A².

In certain embodiments, L is selected from the group consisting of

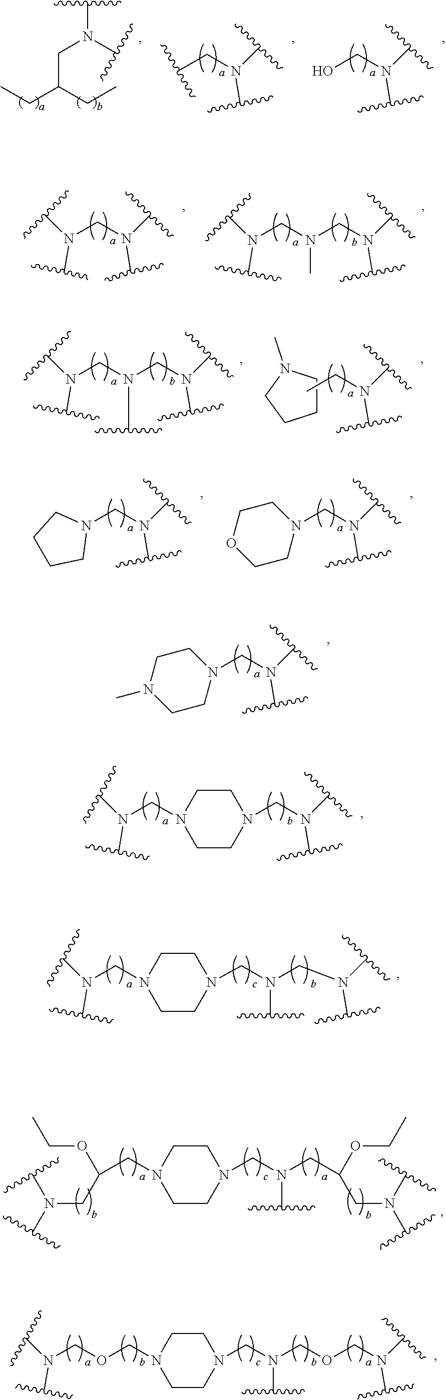

any combination thereof,
wherein:
each occurrence of a, b, and c is independently an integer from 0 to 10; and
each occurrence of

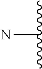 [SMILES: *N]

indicates a bond between a N atom of L and A¹ or A².

In certain embodiments, the compound having the structure of Formula (I) is

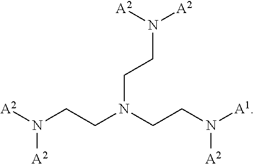

In certain embodiments, the compound having the structure of Formula (I) is

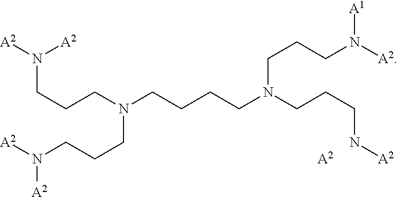

In certain embodiments, the compound having the structure of Formula (I) is

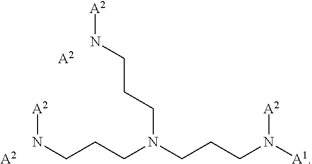

In certain embodiments, the compound having the structure of Formula (I) is

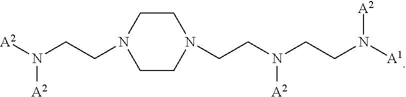

In certain embodiments, the compound having the structure of Formula (I) is

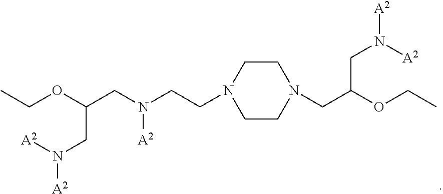

In certain embodiments, the compound having the structure of Formula (T) is

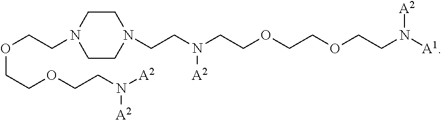

In certain embodiments, the compound having the structure of Formula (I) is

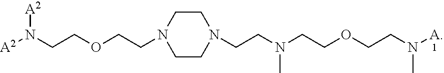

In certain embodiments, A¹ is

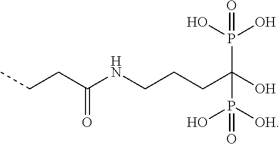

In certain embodiments, A² is

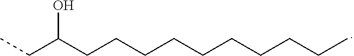

In certain embodiments, A² is

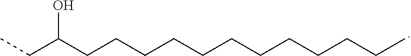

In certain embodiments, A² is

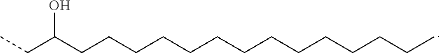

In certain embodiments, the compound having the structure of Formula (I) is a compound having the structure selected from the group consisting of:

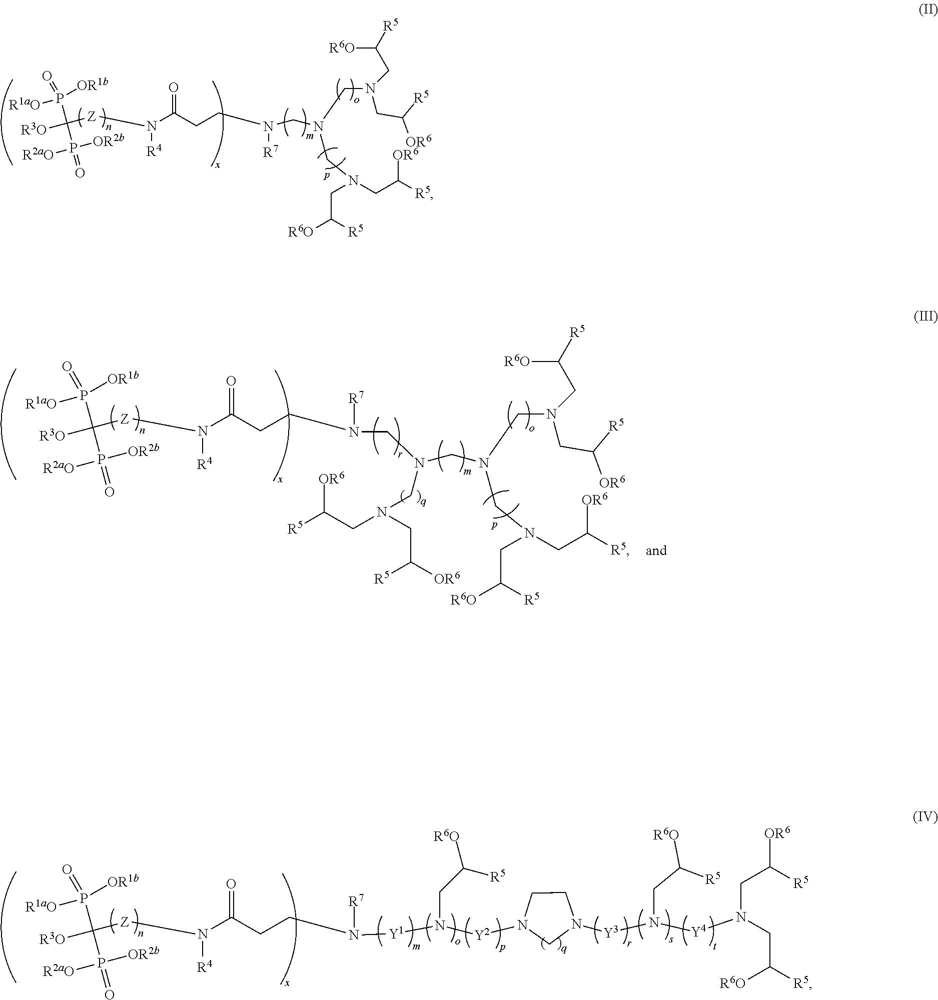

wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R^{1f})_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof;
each occurrence of Y¹, Y², Y³, and Y⁴ is independently selected from the group consisting of O, S, C(R⁹)_{z′}(R¹⁰)_{z″}, N(R⁹)_{z′}, P(R)_{z′}, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5.

In certain embodiments, the compound having the structure of Formula (I) is a compound having the structure selected from the group consisting of:

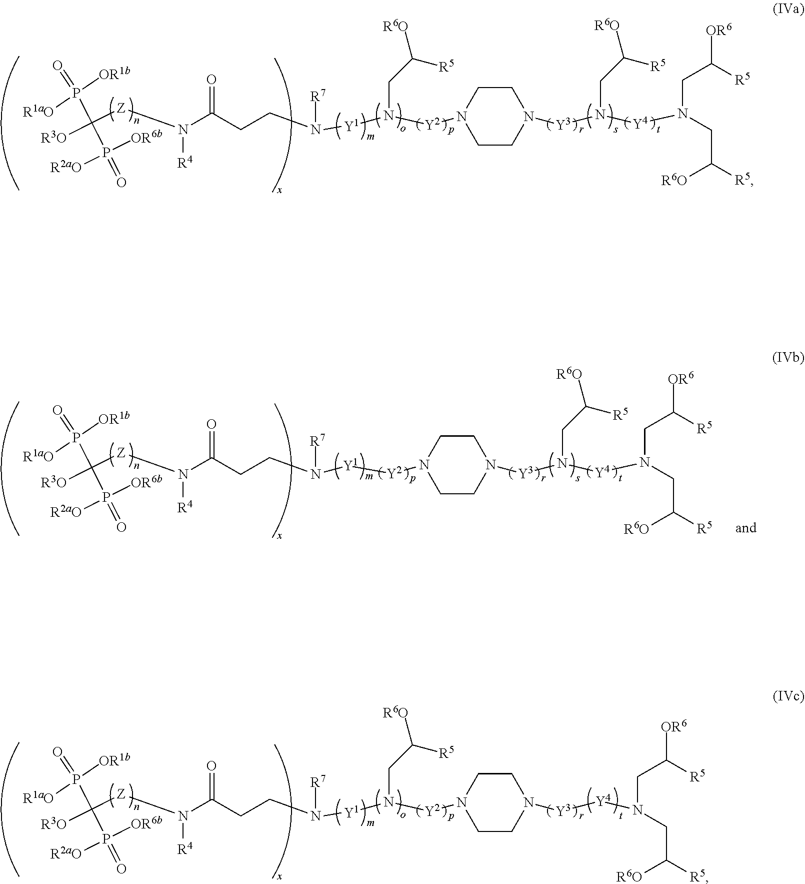

wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴. R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof;
each occurrence of Y¹, Y², Y³, and Y⁴ is independently selected from the group consisting of O, S, C(R⁹)_{z′}(R¹⁰)_{z″}, N(R⁹)_{z′}, P(R⁹)_{z′}, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5.

In certain embodiments, the compound having the structure of Formula (I) is a compound having the structure selected from the group consisting of:

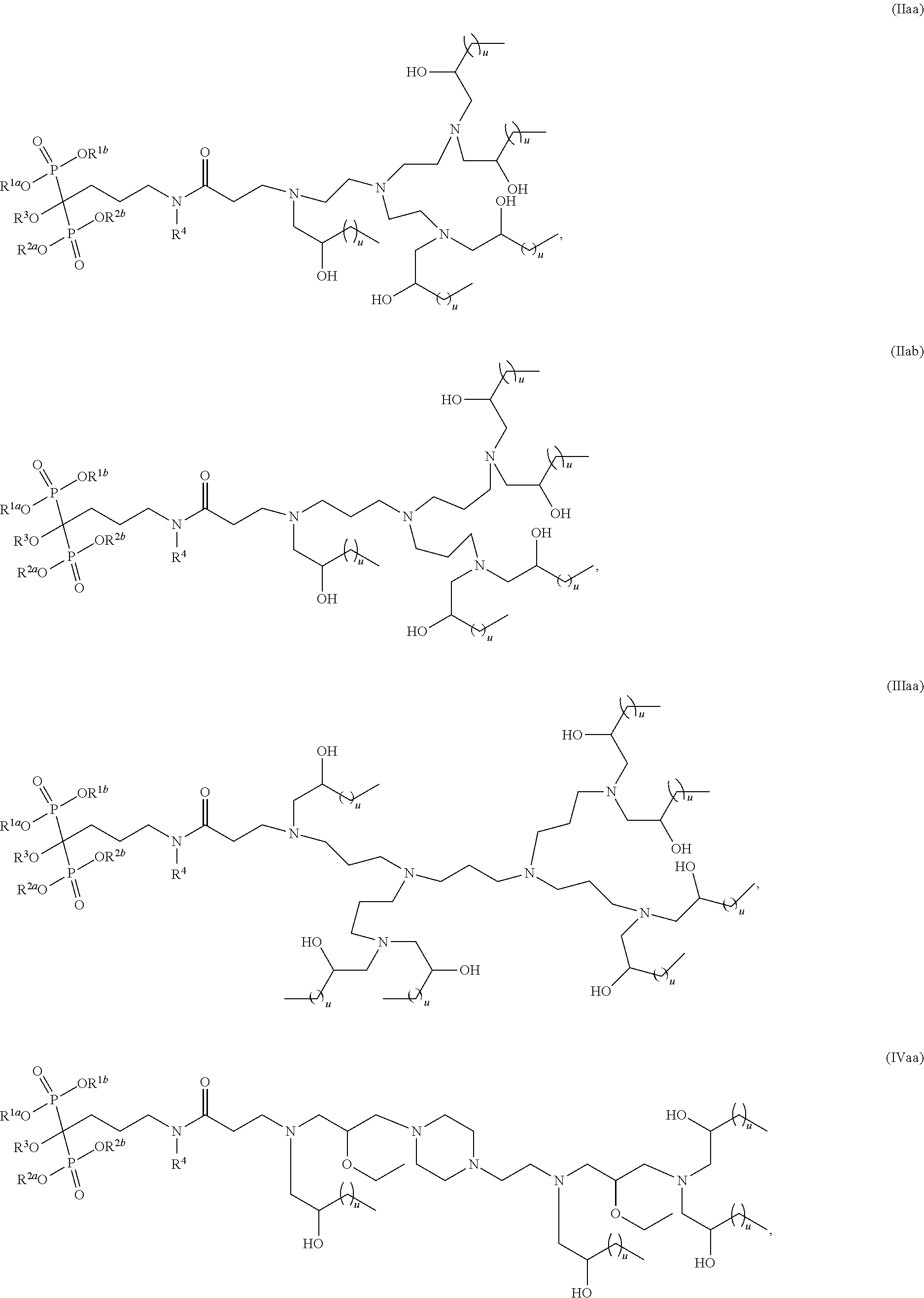

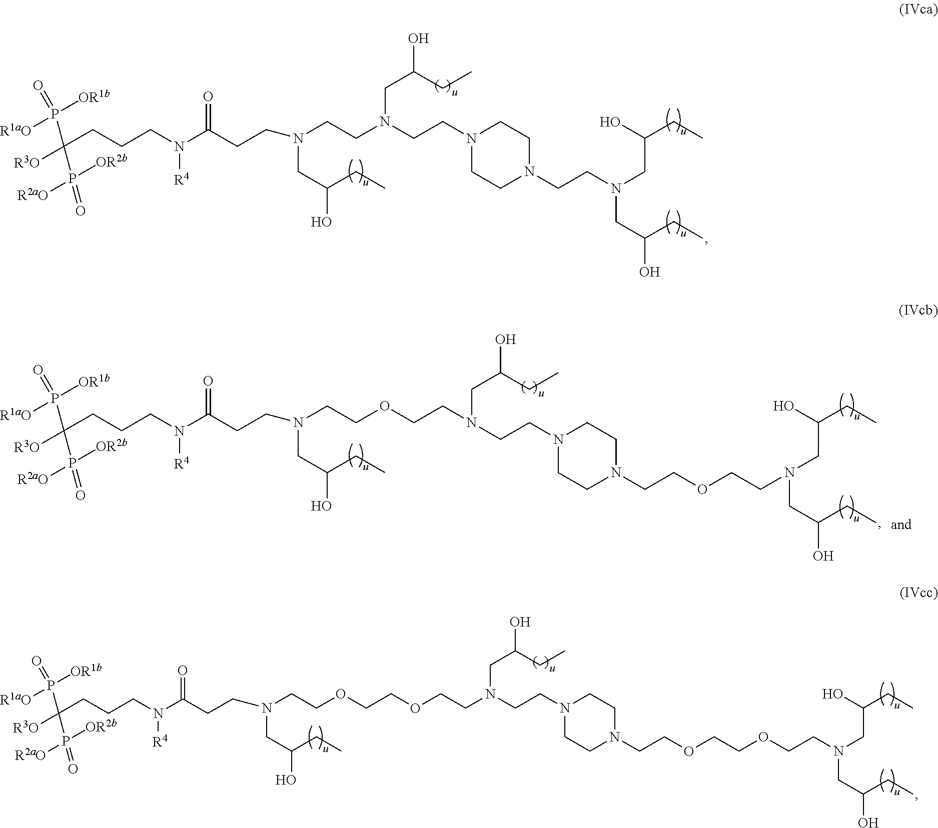

wherein:
each occurrence of R^{1a}, R^{1b}, R^{2a}, R^{2b}, R³, and R⁴ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}-(2-1 heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl. optionally substituted C₈-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S. sulfoxy, sulfonate, sulfate, sulfite, and sulfide, and any combination thereof;
each occurrence of R⁹ and R¹⁰ is independently selected from the group consisting of halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein u is an integer from 0 to 20.

In certain embodiments, the compound having the structure of Formula (I) is selected from the group consisting of

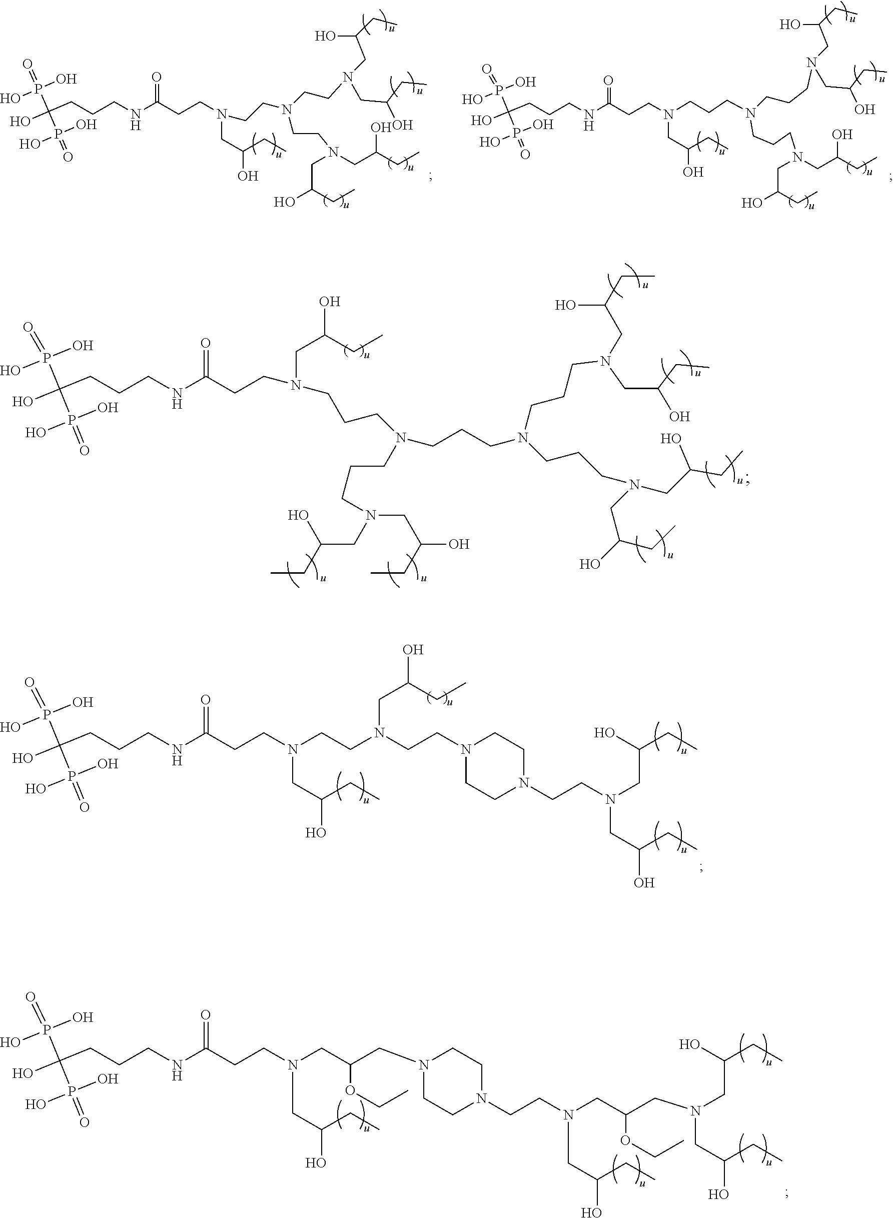

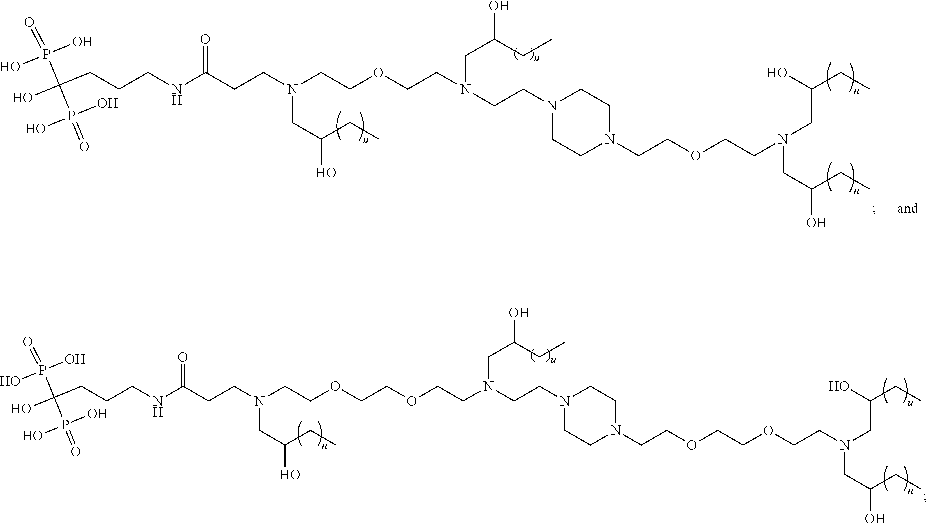

wherein u is an integer from 5 to 15.

In certain embodiments, the compound having the structure of Formula (I) is:

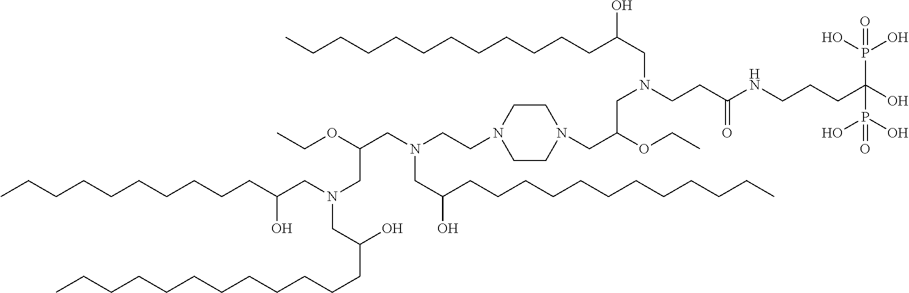 [SMILES: CCCCCCCCCCCCC(O)CN(CCC(=O)NCCCC(O)(P(=O)(O)O)P(=O)(O)O)CC(CN1CCN(CCN(CC(O)CCCCCCCCCCCC)CC(CN(CC(O)CCCCCCCCCC)CC(O)CCCCCCCCCCCC)OCC)CC1)OCC]

(4-(3-((3-(4-(2-((3-(bis(2-hydroxytetradecyl)amino)-2-ethoxypropyl)(2-hydroxytetradecyl)amino)ethyl)piperazin-1-yl)-2-ethoxypropyl)(2-hydroxytetradecyl)amino)propanamido)-1-hydroxybutane-1,1-diyl)bis(phosphonic acid).

### LNP Compositions

In certain embodiments, the cationic lipid is an amino lipid. Suitable amino lipids useful in the disclosure include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In certain embodiments, the lipid is a PEGylated lipid, including, but not limited to, DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoyl-phosphatidylethanolamine (DSPE)-maleimide-PEG, distearoyl-phosphatidylethanolamine (DSPE)-maleimide-PEG2000, 16-O-monomethyl PE, 16-O-dimethyl PE, 18-1-trans PE, 1-stearioyl-2-oleoyl-phosphatidyethanol amine (SOPE), stearoyloleoylphosphatidylcholine (SOPC), and 1,2-dielaidoyl-sn-glycero-3-phophoethanolamine (transDOPE). In certain embodiments, the neutral lipid is 1,2-distearoyl-sn-glycero-3-phosphocholine (DSPC).

In some embodiments, the composition comprises a neutral lipid selected from DSPC, DPPC, DSPE, SOPE, SOPC, DOTAP, DMPC, DOPC, POPC, DOPE, and SM.

A “steroid” is a compound comprising the following carbon skeleton:

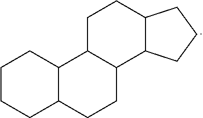 [SMILES: C1CCC2C(C1)CCC1C3CCCC3CCC21]

In certain embodiments, the steroid or steroid analogue is cholesterol. In some of these embodiments, the molar ratio of the cationic lipid.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitovloleyolphosphatidylglycerol (POPG), and other anionic modifying groups joined to neutral lipids.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include polyethylene glycol (PEG), maleimide PEG (mPEG), DSPE-PEG-DBCO, 1-(monomethoxy-polyethyleneglycol)-2,3-dimyristoylglycerol (PEG-s-DMG), DOPE-PEG-Azide, DSPE-PEG-Azide, DPPE-PEG-Azide, DSPE-PEG-Carboxy-NHS, DOPE-PEG-Carboxylic Acid. DSPE-PEG-Carboxylic acid and the like.

In certain embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In certain embodiments, the polyethylene glycol-lipid is N-[(methoxy poly(ethylene glycol)₂₀₀₀)carbamyl]-1,2-dimyristyloxlpropyl-3-amine (PEG-c-DMA). In certain embodiments, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), apegylated phosphatidylethanoloamine (PEG-PE), a PEG succinate diacylglycerol (PEG-S-DAG) such as 4-O-(2′,3′-di(tetradecanoyloxy)propyl-1-O-(w-methoxy(polyethoxy)ethyl)butanedioate (PEG-S-DMG), a pegylated ceramide (PEG-cer), or a PEG dialkoxypropylcarbamate such as w-methoxy(polyethoxy)ethyl-N-(2,3-di(tetradecanoxy)propyl)carbamate or 2,3-di(tetradecanoxy)propyl-N-(w-methoxy(polyethoxy)ethyl)carbamate.

In certain embodiments, the additional lipid is present in the LNP in an amount from about 1 mol % to about 10 mol %. In certain embodiments, the additional lipid is present in the LNP in an amount from about 1 mol % to about 5 mol %. In certain embodiments, the additional lipid is present in the LNP in about 1 mol % or about 2.5 mol %.

The term “lipid nanoparticle” refers to a particle having at least one dimension on the order of nanometers (e.g., 1-1,000 nm) which includes one or more lipids, for example a lipid of Formula (I)—(IV).

In various embodiments, the LNPs have a mean diameter of from about 10 nm to about 1500 nm, about 30 nm to about 1000 nm, about 30 nm to about 500 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 200 nm, 250 nm, 300 nm, 310 nm, 375 nm, 400 nm, 500 nm, 800 nm, 1000 nm, 1250 nm, 1400 nm, or 1500 nm. For example, in some embodiments, the LNPs have a mean diameter of from about 10 nm to about 1000 nm. In some embodiments, the LNPs have a mean diameter of from about 50 nm to about 500 nm.

In various embodiments, the lipids or the LNP of the present disclosure are substantially non-toxic.

In various embodiments, the lipids or the LNPs described herein are formulated for stability for in vivo cell targeting. For example, in some embodiments, the LNP formulated for stability for in vivo cell targeting comprises at least one compound having the structure of Formula (I) in a concentration range of about 0.1 mol % to about 99.99 mol %. In some embodiments, the at least one compound having the structure of Formula (I) is present in concentration range of about 1 mol % to about 45 mol %. In some embodiments, the at least one compound having the structure of Formula (I) is present in a concentration of about 40 mol %. In some embodiments, the at least one compound having the structure of Formula (I) is present in a concentration of about 30 mol %.

In some embodiments, the LNP formulated for stability for in vivo cell targeting comprises a phospholipid in a concentration range of about 10 mol % to about 45 mol %. In certain embodiments, the phospholipid is dioleoyl-phosphatidylethanolamine (DOPE), and the DOPE is present in a molar ratio of about 16 or at a molar percentage of about 16%.

In some embodiments, the LNP formulated for stability for in vivo a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.) targeting comprises total cholesterol lipid in a concentration range of about 5 mol % to about 50 mol %. In certain embodiments, the total cholesterol is present in a molar ratio of about 46.5, or at a molar percentage of about 46.5%.

In some embodiments, the total cholesterol comprises a substituted cholesterol lipid. In some embodiments, the total cholesterol comprises a mixture of cholesterol and one or more substituted cholesterol lipid. In certain embodiments, the LNP molecule comprises total cholesterol at a ratio of 50% cholesterol:50% substituted cholesterol. In certain embodiments, the LNP molecule comprises total cholesterol at a ratio of 75% cholesterol:25% substituted cholesterol. In certain embodiments, the LNP molecule comprises total cholesterol at a ratio of 87.5% cholesterol:12.5% substituted cholesterol. In certain embodiments, the LNP molecule comprises total cholesterol at a ratio of 0% cholesterol:100% substituted cholesterol.

Exemplary substituted cholesterol lipids that can be incorporated into the LNP of the disclosure include, but are not limited to, a hydroxy substituted cholesterol, an epoxy substituted cholesterol and a keto substituted cholesterol.

In some embodiments, the substituted cholesterol lipid is 7α-hydroxycholesterol, 7β-hydroxycholesterol, 19-hydroxycholesterol, 20(S)-hydroxycholesterol, 24(S)-hydroxycholesterol, 25-hydroxycholesterol, 7-ketocholesterol, 5,6-epoxycholesterol, 3β, 5α, 6β-trihydroxycholesterol, 4β-hydroxycholesterol, 27-hydroxycholesterol or 22(R)-hydroxycholesterol.

By way of example, In certain embodiments, the LNP molecule comprises a mixture of 50% cholesterol:50% 7α-hydroxycholesterol. In certain embodiments, the LNP molecule comprises a mixture of 75% cholesterol:25% 7α-hydroxycholesterol.

In some embodiments, the LNP of the present disclosure comprises total PEG in a concentration range of about 0.5 mol % to about 12.5 mol %. In certain embodiments, the total PEG is present in a molar ratio of about 2.5, or at a molar percentage of about 2.5%.

In some embodiments, the PEG comprises a mixture of PEG maleimide PEG (mPEG).

In various embodiments, the LNP of the present disclosure comprises at least one compound having the structure of Formula (I), phospholipid, total cholesterol, and a polymer-conjugated lipid (e.g., PEG-conjugated lipid), wherein the at least one compound having the structure of Formula (I): phospholipid:total cholesterol:polymer-conjugated lipid are present in a molar ratio of about 1-80:5-45:5-55:0.5-12.5 or at a molar percentage of about 1-80%:5-45%:5-55%:0.5-12.5%. In certain embodiments, the LNP comprises at least one compound having the structure of Formula (I), phospholipid, total cholesterol and polymer-conjugated lipid, wherein the at least one compound having the structure of Formula (I): phospholipid:total cholesterol:polymer-conjugated lipid are present in a molar ratio of about 35-45:5-20:40-55:1-2.5 or at a molar percentage of about 35-45%:5-20%:40-55%:1-2.5%. In certain embodiments, the LNP comprises at least one compound having the structure of Formula (I), phospholipid, total cholesterol and polymer-conjugated lipid, wherein the at least one compound having the structure of Formula (I): phospholipid:total cholesterol:polymer-conjugated lipid are present in a molar ratio of about 30-35:16:46.5:2.5 or at a molar percentage of about 35%:16%:46.5%:2.5%. In certain embodiments, the LNP comprises at least one compound having the structure of Formula (I), phospholipid, total cholesterol and polymer-conjugated lipid, wherein the at least one compound having the structure of Formula (I): phospholipid:total cholesterol:polymer-conjugated lipid are present in a molar ratio of about 35:16:46.5:2.5 or at a molar percentage of about 30-35%:16%:46.5%:2.5%.

For example, In certain embodiments, the LNP comprises at least one compound having the structure of Formula (I), DOPE, total cholesterol, and PEG, wherein the at least one compound having the structure of Formula (I):DOPE:total cholesterol:PEG are present in a molar ratio of about 1-80:5-45:5-55:0.5-12.5 or at a molar percentage of about 1-80%:5-45%:5-55%:0.5-12.5%. In certain embodiments, the LNP comprises at least one compound having the structure of Formula (I), DOPE, total cholesterol and PEG, wherein the at least one compound having the structure of Formula (I):DOPE:total cholesterol:PEG are present in a molar ratio of about 35-45:5-20:40-55:1-2.5 or at a molar percentage of about 35-45%:5-20%:40-55%:1-2.5%. In certain embodiments, the LNP comprises at least one compound having the structure of Formula (I), DOPE, total cholesterol and PEG, wherein the at least one compound having the structure of Formula (I):DOPE:total cholesterol:PEG are present in a molar ratio of about 30-35:16:46.5:2.5 or at a molar percentage of about 35%: 16%:46.5%:2.5%. In certain embodiments, the LNP comprises at least one compound having the structure of Formula (I), DOPE, total cholesterol and PEG, wherein the at least one compound having the structure of Formula (I):DOPE:total cholesterol:PEG are present in a molar ratio of about 35:16:46.5:2.5 or at a molar percentage of about 30-35%:16%: 46.5%:2.5%.

In various embodiments, the LNP targets at least one cell of interest. For example, in some embodiments, the LNP targets at least one stem cell, HSC, bone cell, bone marrow cell, or any combination thereof. In some embodiments, the LNP targets at least one stem cell, HSC, stroma cell, osteoblast, osteocyte, osteoclast, bone lining cell, local mesenchymal cell, progenitor cell. mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, or any combination thereof.

In one aspect, the LNP comprises at least one cargo. In various aspects, the disclosure is not limited to any particular cargo or otherwise agent for which the LNP is able to carry or transport. Rather, the disclosure includes any agent that can be carried by the LNP. For example, agents that can be carried by the LNP of the disclosure include, but are not limited to, diagnostic agents, detectable agents, and therapeutic agents. Thus, in various embodiments, the LNP comprises at least one agent. In other embodiments, the LNP encapsulates at least one agent.

In some embodiments, the LNP comprises, or encapsulates, at least one agent. In some embodiments, the weight ratio of (a): the at least one agent is between about 1:1 to about 10:1. In some embodiments, the LNP comprises, or encapsulates, at least one agent. In some embodiments, the weight ratio of (a): the at least one agent is between about 2:1 to about 10:1. In some embodiments, the LNP comprises, or encapsulates, at least one agent. In some embodiments, the weight ratio of (a): the at least one agent is between about 3:1 to about 10:1. In some embodiments, the LNP comprises, or encapsulates, at least one agent. In some embodiments, the weight ratio of (a): the at least one agent is between about 4:1 to about 10:1. In some embodiments, the LNP comprises, or encapsulates, at least one agent. In some embodiments, the weight ratio of (a): the at least one agent is between about 5:1 to about 10:1. In some embodiments, the LNP comprises, or encapsulates, at least one agent. In some embodiments, the weight ratio of (a): the at least one agent is between about 6:1 to about 10:1. In some embodiments, the LNP comprises, or encapsulates, at least one agent. In some embodiments, the weight ratio of (a): the at least one agent is between about 7:1 to about 10:1. In some embodiments, the LNP comprises, or encapsulates, at least one agent. In some embodiments, the weight ratio of (a): the at least one agent is between about 8:1 to about 10:1. In some embodiments, the LNP comprises, or encapsulates, at least one agent. In some embodiments, the weight ratio of (a): the at least one agent is between about 9:1 to about 10:1. In some embodiments, the LNP comprises, or encapsulates, at least one agent. In some embodiments, the weight ratio of (a): the at least one agent is between about 9.5:1 to about 10:1.

Thus, in various embodiments, the LNP is suitable for delivering at least one cargo to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.).

In one aspect, the present disclosure relates to a composition comprising at least one compound or LNP of the present disclosure. In one aspect, the present disclosure relates to a composition comprising at least one compound or LNP of the present disclosure that selectively targets at least one cell of interest. For example, in some embodiments, the composition targets at least one bone cell and/or bone marrow cell (e.g., a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.).

In one aspect, the composition of the present disclosure comprises one or more LNP formulated for targeted delivery of an agent to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.). Examples of such agents include, but are not limited to, a therapeutic agent, diagnostic agent, detectable agent, small molecule, peptide, polypeptide, amino acid molecule, nucleic acid molecule, drug, pro-drug, label, or any combination thereof.

For example, in some embodiments, the composition of the present disclosure comprises at least one therapeutic agent. In certain embodiments, the therapeutic agent is a hydrophobic therapeutic agent. In certain embodiments, the therapeutic agent is a hydrophilic therapeutic agent. Examples of such therapeutic agents include, but are not limited to, one or more drugs, proteins, amino acids, peptides, antibodies, antibiotics, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

In certain embodiments, the therapeutic agent is one or more non-therapeutic moieties. In some embodiments, the nanoparticle comprises one or more therapeutic moieties, one or more non-therapeutic moieties, or any combination thereof. In certain embodiments, the therapeutic moiety targets cancer. In some embodiments, the composition comprises folic acid, peptides, proteins, aptamers, antibodies, small RNA molecules, niRNA, shRNA, siRNA, poorly water-soluble therapeutic agents, anti-cancer agents, or any combinations thereof.

In certain embodiments, the therapeutic agent may be an anti-cancer agent. Any suitable anti-cancer agent may be used in the compositions and methods of the present disclosure. The selection of a suitable anti-cancer agent may depend upon, among other things, the type of cancer to be treated and the nanoparticle compositions of the present disclosure. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. Examples of anti-cancer agents include, but is not limited to, chemotherapeutic agents, antiproliferative agents, anti-tumor agents, checkpoint inhibitors, and anti-angiogenic agents. For example, in certain embodiments, the anti-cancer agent is gemcitabine, doxorubicin, 5-Fu, tyrosine kinase inhibitors, sorafenib, trametinib, rapamycin, fulvestrant, ezalutamide, or paclitaxel.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26. navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene). Additional examples of specific antiproliferative agents include, but are not limited to levamisole, gallium nitrate, granisetron, sargramostim strontium-89 chloride, filgrastim, pilocarpine, dexrazoxane, and ondansetron.

The inhibitors of the disclosure can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; my cobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In certain embodiments, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

In some embodiments, the LNP further comprises one or more gene components, such as siRNA or therapeutic DNA fragments. In some embodiments, the gene component is encapsulated in the LNP. In some embodiments, the gene component is on the surface of the LNP, for example, attached to or within the coating material.

In some embodiments, the LNP further comprises a biocompatible metal. Examples of biocompatible metals include, but are not limited to, copper, copper sulfide, iron oxide, cobalt and noble metals, such as gold and/or silver. One of ordinary skill in the art will be able to select of a suitable type of LNP taking into consideration at least the type of imaging and/or therapy to be performed.

### Small Molecule

In various embodiments, the agent is a small molecule. In various embodiments, the agent is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis, and in vitro translation systems, using methods well known in the art. In certain embodiments, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the disclosure, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the disclosure, the therapeutic agent is synthesized via small library synthesis.

The small molecule and small molecule compounds described herein may be present as salts even if salts are not depicted, and it is understood that the disclosure embraces all salts and solvates of the therapeutic agents depicted here, as well as the non-salt and non-solvate form of the therapeutic agents, as is well understood by the skilled artisan. In some embodiments, the salts of the therapeutic agents of the disclosure are pharmaceutically acceptable salts.

Where tautomeric forms may be present for any of the therapeutic agents described herein, each and every tautomeric form is intended to be included in the present disclosure, even though only one or some of the tautomeric forms may be explicitly depicted. For example, when a 2-hydroxypyridyl moiety is depicted, the corresponding 2-pyridone tautomer is also intended.

The disclosure also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the disclosure, such as crystalline or non-crystalline forms of the therapeutic agent. Compositions comprising a therapeutic agents of the disclosure are also intended, such as a composition of substantially pure therapeutic agent, including a specific stereochemical form thereof, or a composition comprising mixtures of therapeutic agents of the disclosure in any ratio, including two or more stereochemical forms, such as in a racemic or non-racemic mixture.

The disclosure also includes any or all active analog or derivative, such as a prodrug, of any therapeutic agent described herein. In certain embodiments, the therapeutic agent is a prodrug. In certain embodiments, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the present disclosure can be used to treat a disease, disorder, or condition.

In certain embodiments, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Molecule

In other related aspects, the agent is a nucleic acid molecule. In various embodiments, the agent is an isolated nucleic acid. Thus, in certain embodiments, an isolated nucleic acid, including for example a DNA oligonucleotide and a RNA oligonucleotide can be incorporated in the composition of the disclosure. In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a cDNA, mRNA, siRNA, shRNA or miRNA molecule. In certain embodiments, the isolated nucleic acid molecule encodes a therapeutic peptide such a thrombomodulin, endothelial protein C receptor (EPCR), anti-thrombotic proteins including plasminogen activators and their mutants, antioxidant proteins including catalase, superoxide dismutase (SOD) and iron-sequestering proteins. In some embodiments, the therapeutic agent is an siRNA, miRNA, shRNA, or an antisense molecule, which inhibits a targeted nucleic acid including those encoding proteins that are involved in aggravation of the pathological processes.

In certain embodiments, the nucleic acid comprises a promoter/regulatory sequence such that the nucleic acid is capable of directing expression of the nucleic acid. Thus, the disclosure encompasses expression vectors and methods for the introduction of exogenous nucleic acid into cells with concomitant expression of the exogenous nucleic acid in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, PA (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216.

In one aspect, the disclosure includes a vector comprising an siRNA or an antisense polynucleotide. Preferably, the siRNA or antisense polynucleotide is capable of inhibiting the expression of a target polypeptide. The incorporation of a desired polynucleotide into a vector and the choice of vectors are well-known in the art as described in, for example, Sambrook et al. (2012), and in Ausubel et al. (1997), and elsewhere herein.

In certain embodiments, the expression vectors described herein encode a short hairpin RNA (shRNA) therapeutic agents. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using a delivery vehicle of the disclosure. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one aspect, the delivery vehicle may contain a vector, comprising the nucleotide sequence or the construct to be delivered. The choice of the vector will depend on the host cell in which it is to be subsequently introduced. In a particular embodiment, the vector of the disclosure is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present disclosure to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the disclosure or the gene construct of the disclosure can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In certain embodiments, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like.

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queuosine, and wybutosine and the like, as well as acetyl- methyl-, thio- and other modified forms of adenine, cytidine, guanine, thymine, and uridine.

In certain embodiments of the disclosure, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the disclosure may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the disclosure include oligonucleotide derivatives known in the art which have improved biological activity compared to unmodified oligonucleotides (see U.S. Pat. No. 5,023,243).

In certain embodiments of the disclosure, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, CA) or they may be genetically expressed from DNA encoding them.

In certain embodiments, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In certain embodiments, the therapeutic agent comprises a gRNA or a nucleic acid molecule encoding a gRNA. In certain embodiments, the therapeutic agent comprises a Cas peptide or a nucleic acid molecule encoding a Cas peptide.

In certain embodiments, the agent comprises a miRNA or a mimic of a miRNA. In certain embodiments, the agent comprises a nucleic acid molecule that encodes a miRNA or mimic of a miRNA.

miRNAs are small non-coding RNA molecules that are capable of causing post-transcriptional silencing of specific genes in cells by the inhibition of translation or through degradation of the targeted mRNA. A miRNA can be completely complementary or can have a region of non-complementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822. and 2005028824, each of which is hereby incorporated by reference in its entirety. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3-terminus can be blocked with an aninoalkyl group. Other 3′ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In certain embodiments, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅Q. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells (see, e.g., U.S. Pat. Nos. 5,656,611, 5,753,613, 5,785,992, 6,120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a maimer that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In certain embodiments, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of a miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In certain embodiments, the oligonucleotide shares identity with endogenous miRNA or miRNA precursor nucleobase sequences. An oligonucleotide selected for inclusion in a composition of the present disclosure may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present disclosure encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In the sense used in this description, a nucleotide sequence is “substantially homologous” to any of the nucleotide sequences describe herein when its nucleotide sequence has a degree of identity with respect to the nucleotide sequence of at least 60%, advantageously of at least 70%, preferably of at least 85%, and more preferably of at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. The identity between two amino acid sequences is preferably determined by using the BLASTN algorithm.

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

### Polypeptide

In other related aspects, the agent is a polypeptide. In various embodiments, the agent is an isolated polypeptide. In other related aspects, the therapeutic agent includes an isolated polypeptide. For example, in certain embodiments, the polypeptide of the disclosure inhibits or activates a target directly by binding to the target thereby modulating the normal functional activity of the target. In certain embodiments, the polypeptide of the disclosure modulates the target by competing with endogenous proteins. In certain embodiments, the polypeptide of the disclosure modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present disclosure, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

In one aspect, the disclosure includes an ionizable LNP molecule comprising or encapsulating one or more agent (e.g., a nucleic acid molecule) for targeted in vivo delivery of the encapsulated agent to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.). In certain embodiments, the nucleic acid molecule is a mRNA molecule.

In some embodiments, the mRNA molecule comprises a nucleotide sequence that can alternatively comprise sequence variations with respect to the original nucleotide sequences, for example, substitutions, insertions and/or deletions of one or more nucleotides, with the condition that the resulting polynucleotide encodes a polypeptide according to the disclosure.

As used herein, an amino acid sequence is “substantially homologous” to any of the amino acid sequences described herein when its amino acid sequence has a degree of identity with respect to the amino acid sequence of at least 60%, advantageously of at least 70%, preferably of at least 85%, and more preferably of at least 95%. The identity between two amino acid sequences is preferably determined by using the BLASTN algorithm.

In certain embodiments, the composition comprises a plurality of constructs, each construct encoding one or more antigens. In certain embodiments, the composition comprises 1 or more, 2 or more, 5 or more, 10 or more, 15 or more, or 20 or more constructs. In certain embodiments, the composition comprises a first construct, comprising a nucleotide sequence encoding an antigen; and a second construct, comprising a nucleotide sequence encoding an adjuvant.

In certain embodiments, the construct comprises a plurality of nucleotide sequences encoding a plurality of antigens. In certain embodiments, the construct encodes 1 or more, 2 or more, 5 or more, 10 or more, 15 or more, or 20 or more antigens. In certain embodiments, the disclosure relates to a construct, comprising a nucleotide sequence encoding an adjuvant. For example, in certain embodiments, the construct comprises a first nucleotide sequence encoding an antigen and a second nucleotide sequence encoding an adjuvant.

In another particular embodiment, the construct is operatively bound to a translational control element. The construct can incorporate an operatively bound regulatory sequence for the expression of the nucleotide sequence of the disclosure, thus forming an expression cassette.

### Peptides

In certain embodiments, the agent is a peptide. Thus, in one aspect, a peptide can be incorporated into the LNP. Thus, in certain embodiments, the agent is a peptide. The peptide of the present disclosure may be made using chemical methods. For example, peptides can be synthesized by solid phase techniques, cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present disclosure may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present disclosure, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present disclosure includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm.

The peptides of the disclosure can be post-translationally modified. For example, post-translational modifications that fall within the scope of the present disclosure include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the disclosure may include unnatural amino acids formed by post-translational modification or by introducing unnatural amino acids during translation.

### Antibodies

In certain embodiments, the agent is an antibody. Thus, in various embodiments, the composition of the disclosure comprises an antibody, or antibody fragment. In certain embodiments, the antibody targeting domain specifically binds to a target of interest. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule (Ladner et al., U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

### Chimeric Antigen Receptor (CAR) Agents

In certain embodiments, the agent comprises a nucleic acid sequence encoding a chimeric antigen receptor (CAR). In certain embodiments, the agent comprises an mRNA molecule encoding a CAR. In certain embodiments, the agent comprises a modified nucleoside mRNA molecule encoding a CAR.

In certain embodiments, a CAR comprises an extracellular domain capable of binding an antigen, including a tumor or pathogen antigen.

Targets of antigen-specific targeting regions of CARs may be of any kind. In some embodiments, the antigen-specific targeting region of the CAR targets antigens specific for cancer, inflammatory disease, neuronal-disorders, diabetes, cardiovascular disease, infectious diseases or a combination thereof. Examples of antigens that may be targeted by the CARs include but are not limited to antigens expressed on B-cells, antigens expressed on carcinomas, sarcomas, lymphomas, leukemia, germ cell tumors, blastomas, antigens expressed on various immune cells, and antigens expressed on cells associated with various hematologic diseases, autoimmune diseases, and/or inflammatory diseases. The CARs of the disclosure may be capable of redirecting the effector function of the expressing-cells to the target antigen(s).

Antigens that may be targeted by the CARs of the disclosure include but are not limited to any one or more of 4-IBB, 707-AP, 5T4, adenocarcinoma antigen, alpha-fetoprotein, BAFF, B-lymphoma cell, C242 antigen, CA-125, carbonic anhydrase 9 (CA-IX), C-MET, ART-4, BAGE, b-catenin/m, bcr-abl, CAMEL, CAP-1, CCR4, CD 152, CD7, CD 19, CD20, CD200, CD22, CD221, CD23 (IgE receptor), CD28, CD30 (TNFRSF8), CD33, CD4, CD38, CD40, CD44 v6, CD44v7/8, CD51, CD52, CD56, CD74, CD80, CD93, CD123, CD171, CEA, CLPP, CNT0888, CTLA-4, carcinoembryonic antigen, EGP2, EGP40, DR5, ErbB2, ErbB3/4, EGFR, EpCAM, EPV-E6, CD3, CASP-8, CD109, CDK/4, CDC-27, Cyp-B, DAM-8, DAM-10, ELV-M2, ETV6, FAP, fibronectin extra domain-B, folate receptor 1, GAGE, GD2, GD3 ganglioside, glycoprotein 75, GPNMB, G250, Gp100, HAGE, HER2/neu, HGF, HMW-MAA, human scatter factor receptor kinase, hTERT, IGF-1 receptor, IGF-I, IgG1, —I-CAM, IL-13, IL-6, insulin-like growth factor I receptor, integrin α5β1, integrin αvβ, Kappa or light chain, LAGE, Lewis Y, G250/CAIX, Glypican-3, MAGE, MC1-R, mesothelin, MORAb-009, MS4A1, MUC1, MUC16, mucin CanAg, N-glycolylneuraminic acid, NPC-1C, PDGF-R a, PDL192, phosphatidylserine, PSC1, PSMA, NKG2D ligands, RANKL, RON, ROR1, SAGE, SCH 900105, SDC1, SLAMF7, TAG-72, TEL/AML, tenascin C, TGF beta 2, TGF-β, TRAIL-R1, TRAIL-R2, tumor antigen CTAA16.88, VEGF-A, VEGFR-1, VEGFR2, vimentin, B7-H6, IL-13 receptor a2, IL-11 receptor Ra, 8H9, NCAM, Fetal AchR, iCE, MART-1, tyrosinase, WT-1, TEM-1, TEM-2, TEM-3, TEM-4, TEM-5, TEM-6, TEM-7, TEM-8, ROBO-4, and so forth. Other antigens specific for cancer will be apparent to those of skill in the art and may be used in connection with alternate embodiments of the disclosure.

Particular examples of target antigens include but are not limited to surface proteins found on cancer cells in a specific or amplified fashion (e.g. the IL-14 receptor, CD 19, CD20 and CD40 for B-cell lymphoma, the Lewis Y and CEA antigens for a variety of carcinomas, the Tag72 antigen for breast and colorectal cancer, EGF-R for lung cancer, folate binding protein and the HER-2 protein that is often amplified in human breast and ovarian carcinomas), or viral proteins (e.g. gp120 and gp41 envelope proteins of HIV, envelope proteins from the Hepatitis B and C viruses, the glycoprotein B and other envelope glycoproteins of human cytomegalovirus, the envelope proteins from oncoviruses such as Kaposi's sarcoma-associated Herpes virus). Other targets of the CARs of the disclosure include CD4, where the ligand is the HIV gp120 envelope glycoprotein, and other viral receptors, for example ICAM, which is the receptor for the human rhinovirus, and the related receptor molecule for poliovirus.

In some embodiments, the bispecific chimeric antigen receptors target and bind at least two different antigens. Examples of pairings of at least two antigens bound by the bispecific CARs of the disclosure include but are not limited to any combination with HER2, CD 19 and CD20, CD 19 and CD22, CD20 and —I-CAM, —I-CAM and GD2, EGFR and —I-CAM, EGFR and C-MET, EGFR and HER2, C-MET and HER2 and EGFR and ROR1. Other pairings of antigens specific for cancer will be apparent to those of skill in the art and may be used in connection with alternate embodiments of the disclosure. In yet other embodiments, the bispecific chimeric antigen receptor targets CD 19 and CD20.

Antigens specific for inflammatory diseases that may be targeted by the CARs of the disclosure include but are not limited to any one or more of AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ. IgE, IgE Fc region, IL-1, IL-12, IL-23, IL-13, IL-17, IL-17A, IL-22, IL-4, IL-5, IL-5, IL-6, IL-6 receptor, integrin a4, integrin α4β7, Lama glama, LFA-1 (CD11a), MEDI-528, myostatin, OX-40, rhuMAb (37, scleroscin, SOST, TGF beta 1, TNF-α or VEGF-A. Other antigens specific for inflammatory diseases will be apparent to those of skill in the art and may be used in connection with alternate embodiments of the disclosure.

Antigens specific for neuronal disorders that may be targeted by the CARs of the disclosure include but are not limited to any one or more of beta amyloid or MABT5102A. Other antigens specific for neuronal disorders will be apparent to those of skill in the art and may be used in connection with alternate embodiments of the disclosure.

Antigens specific for diabetes that may be targeted by the CARs of the disclosure include but are not limited to any one or more of L-43 or CD3. Other antigens specific for diabetes or other metabolic disorders will be apparent to those of skill in the art and may be used in connection with alternate embodiments of the disclosure.

Antigens specific for cardiovascular diseases which may be targeted by the CARs of the disclosure include but are not limited to any one or more of C5, cardiac myosin, CD41 (integrin alpha-lib), fibrin II, beta chain, ITGB2 (CD 18) and sphingosine-1-phosphate. Other antigens specific for cardiovascular diseases will be apparent to those of skill in the art and may be used in connection with alternate embodiments of the disclosure.

Antigens specific for infectious diseases that may be targeted by the CARs of the disclosure include but are not limited to any one or more of anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus and TNF-α. Other antigens specific for infectious diseases will be apparent to those of skill in the art and may be used in connection with alternate embodiments of the disclosure.

Additional targets of the CARs of the disclosure include antigens involved in B-cell associated diseases. Yet further targets of the CARs of the disclosure will be apparent to those of skill in the art and may be used in connection with alternate embodiments of the disclosure.

Other antigens specific for cancer will be apparent to those of skill in the art and may be used in connection with alternate embodiments of the disclosure.

In certain embodiments, the CAR comprises an antigen binding domain. In a particular non-limiting embodiment, the antigen-binding domain is an scFv specific for binding to a surface antigen of a target cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.).

In various embodiments, the CAR can be a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the disclosure comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3ζ-chain, which is the primary transmitter of signals from endogenous T cell receptors (TCRs). “First generation” CARs can provide de novo antigen recognition and cause activation of both CD4+ and CD8+ T cells through their CD3ζ chain signaling domain in a single fusion molecule, independent of HLA-mediated antigen presentation.

“Second-generation” CARs for use in the disclosure comprise an antigen binding domain, for example, a single-chain variable fragment (scFv), fused to an intracellular signaling domain capable of activating T cells and a co-stimulatory domain designed to augment T cell potency and persistence. CAR design can therefore combine antigen recognition with signal transduction, two functions that are physiologically bome by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD3ζ signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2Rβ.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above.

### Adjuvant

In certain embodiments, the agent is an adjuvant. Thus, in various embodiments, the composition comprises an adjuvant. In certain embodiments, the composition comprises a nucleic acid molecule encoding an adjuvant. In certain embodiments, the adjuvant-encoding nucleic acid molecule is IVT RNA. In certain embodiments, the adjuvant-encoding nucleic acid molecule is nucleoside-modified mRNA.

Exemplary adjuvants include, but is not limited to, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig and functional fragments thereof.

Alternatively, one or more of the agents below is delivered for therapeutic purposes as a sole agent and is not intended to function as an adjuvant to a co-administered compound. For example, the coding sequence for a gene therapy (e.g., replacement) of a desired protein may an agent delivered via an LNP as provided herein. Additionally, the coding sequence for a gene editing enzyme may be delivered. In these and other instances, the LNP may be formulated to minimize any immune response to the agent.

### Nucleoside-Modified RNA

In certain embodiments, the agent is a nucleoside-modified RNA. Thus, in one aspect, the composition comprises a nucleoside-modified RNA. Thus, in certain embodiments, the agent is a nucleoside-modified RNA In certain embodiments, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present disclosure is further described in U.S. Pat. No. 8,278,036, which is incorporated by reference herein in its entirety.

In certain embodiments, nucleoside-modified mRNA does not activate any pathophysiologic pathways, translates very efficiently and almost immediately following delivery, and serve as templates for continuous protein production in vivo lasting for several days. The amount of mRNA required to exert a physiological effect is small and that makes it applicable for human therapy.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable.

It has been demonstrated that the presence of modified nucleosides, including pseudouridines in RNA suppress their innate immunogenicity. Further, protein-encoding, in vitro-transcribed RNA containing pseudouridine can be translated more efficiently than RNA containing no or other modified nucleosides. Subsequently, it is shown that the presence of pseudouridine improves the stability of RNA and abates both activation of PKR and inhibition of translation. A preparative HPLC purification procedure has been established that was critical to obtain pseudouridine-containing RNA that has superior translational potential and no innate immunogenicity. Administering HPLC-purified, pseudourine-containing RNA coding for erythropoietin into mice and macaques resulted in a significant increase of serum EPO levels, thus confirming that pseudouridine-containing mRNA is suitable for in vivo protein therapy.

The present disclosure encompasses RNA, oligoribonucleotide, and polyribonucleotide molecules comprising pseudouridine or a modified nucleoside. In certain embodiments, the composition comprises an isolated nucleic acid encoding an antigen or antigen binding molecule, wherein the nucleic acid comprises a pseudouridine or a modified nucleoside. In certain embodiments, the composition comprises a vector, comprising an isolated nucleic acid encoding an antigen, an antigen binding molecule, an adjuvant, or combination thereof, wherein the nucleic acid comprises a pseudouridine or a modified nucleoside.

In certain embodiments, the nucleoside-modified RNA of the disclosure is IVT RNA. For example, in certain embodiments, the nucleoside-modified RNA is synthesized by T7 phage RNA polymerase. In certain embodiments, the nucleoside-modified mRNA is synthesized by SP6 phage RNA polymerase. In certain embodiments, the nucleoside-modified RNA is synthesized by T3 phage RNA polymerase.

In certain embodiments, the modified nucleoside is m¹acp³Ψ (1-methyl-3-(3-amino-3-carboxypropyl) pseudouridine. In certain embodiments, the modified nucleoside is m¹Ψ (1-methylpseudouridine). In certain embodiments, the modified nucleoside is Ψm (2′-O-methylpseudouridine. In certain embodiments, the modified nucleoside is m⁵D (5-methyldihydrouridine). In certain embodiments, the modified nucleoside is m³Ψ (3-methylpseudouridine). In certain embodiments, the modified nucleoside is a pseudouridine moiety that is not further modified. In certain embodiments, the modified nucleoside is a monophosphate, diphosphate, or triphosphate of any of the above pseudouridines. In certain embodiments, the modified nucleoside is any other pseudouridine-like nucleoside known in the art.

In certain embodiments, the modified nucleoside of the present disclosure is m⁵C (5-methylcytidine). In certain embodiments, the modified nucleoside is m⁵U (5-methyluridine). In certain embodiments, the modified nucleoside is m⁶A (N⁶-methyladenosine). In certain embodiments, the modified nucleoside is s²U (2-thiouridine). In certain embodiments, the modified nucleoside is Ψ (pseudouridine). In certain embodiments, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G¹ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m²⁷G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In certain embodiments, a nucleoside-modified RNA of the present disclosure comprises a combination of 2 or more of the above modifications. In certain embodiments, the nucleoside-modified RNA comprises a combination of 3 or more of the above modifications. In certain embodiments, the nucleoside-modified RNA comprises a combination of more than 3 of the above modifications.

In certain embodiments, between 0.1% and 100% of the residues in the nucleoside-modified of the present disclosure are modified (e.g. either by the presence of pseudouridine or a modified nucleoside base). In certain embodiments, 0.1% of the residues are modified. In certain embodiments, the fraction of modified residues is 0.2%. In certain embodiments, the fraction is 0.3%. In certain embodiments, the fraction is 0.4%. In certain embodiments, the fraction is 0.5%. In certain embodiments, the fraction is 0.6%. In certain embodiments, the fraction is 0.8%. In certain embodiments, the fraction is 1%. In certain embodiments, the fraction is 1.5%. In certain embodiments, the fraction is 2%. In certain embodiments, the fraction is 2.5%. In certain embodiments, the fraction is 3%. In certain embodiments, the fraction is 4%. In certain embodiments, the fraction is 5%. In certain embodiments, the fraction is 6%. In certain embodiments, the fraction is 8%. In certain embodiments, the fraction is 10%. In certain embodiments, the fraction is 12%. In certain embodiments, the fraction is 14%. In certain embodiments, the fraction is 16%. In certain embodiments, the fraction is 18%. In certain embodiments, the fraction is 20%. In certain embodiments, the fraction is 25%. In certain embodiments, the fraction is 30%. In certain embodiments, the fraction is 35%. In certain embodiments, the fraction is 40%. In certain embodiments, the fraction is 45%. In certain embodiments, the fraction is 50%. In certain embodiments, the fraction is 60%. In certain embodiments, the fraction is 70%. In certain embodiments, the fraction is 80%. In certain embodiments, the fraction is 90%. In certain embodiments, the fraction is 100%.

In certain embodiments, the fraction is less than 5%. In certain embodiments, the fraction is less than 3%. In certain embodiments, the fraction is less than 1%. In certain embodiments, the fraction is less than 2%. In certain embodiments, the fraction is less than 4%. In certain embodiments, the fraction is less than 6%. In certain embodiments, the fraction is less than 8%. In certain embodiments, the fraction is less than 10%. In certain embodiments, the fraction is less than 12%. In certain embodiments, the fraction is less than 15%. In certain embodiments, the fraction is less than 20%. In certain embodiments, the fraction is less than 30%. In certain embodiments, the fraction is less than 40%. In certain embodiments, the fraction is less than 50%. In certain embodiments, the fraction is less than 60%. In certain embodiments, the fraction is less than 70%.

In certain embodiments, 0.1t % of the residues of a given nucleoside (i.e., uridine, cytidine, guanosine, or adenosine) are modified. In certain embodiments, the fraction of the given nucleotide that is modified is 0.2%. In certain embodiments, the fraction is 0.3%. In certain embodiments, the fraction is 0.4%. In certain embodiments, the fraction is 0.5%. In certain embodiments, the fraction is 0.6%. In certain embodiments, the fraction is 0.8%. In certain embodiments, the fraction is 1%. In certain embodiments, the fraction is 1.5%. In certain embodiments, the fraction is 2%. In certain embodiments, the fraction is 2.5%. In certain embodiments, the fraction is 3%. In certain embodiments, the fraction is 4%. In certain embodiments, the fraction is 5%. In certain embodiments, the fraction is 6%. In certain embodiments, the fraction is 8%. In certain embodiments, the fraction is 10%. In certain embodiments, the fraction is 12%. In certain embodiments, the fraction is 14%. In certain embodiments, the fraction is 16%. In certain embodiments, the fraction is 18%. In certain embodiments, the fraction is 20%. In certain embodiments, the fraction is 25%. In certain embodiments, the fraction is 30%. In certain embodiments, the fraction is 35%. In certain embodiments, the fraction is 40%. In certain embodiments, the fraction is 45%. In certain embodiments, the fraction is 50%. In certain embodiments, the fraction is 60%. In certain embodiments, the fraction is 70%. In certain embodiments, the fraction is 80%. In certain embodiments, the fraction is 90%. In certain embodiments, the fraction is 100%.

In certain embodiments, the fraction of the given nucleotide that is modified is less than 8%. In certain embodiments, the fraction is less than 10%. In certain embodiments, the fraction is less than 5%. In certain embodiments, the fraction is less than 3%. In certain embodiments, the fraction is less than 1%. In certain embodiments, the fraction is less than 2%. In certain embodiments, the fraction is less than 4%. In certain embodiments, the fraction is less than 6%. In certain embodiments, the fraction is less than 12%. In certain embodiments, the fraction is less than 15%. In certain embodiments, the fraction is less than 20%. In certain embodiments, the fraction is less than 30%. In certain embodiments, the fraction is less than 40%. In certain embodiments, the fraction is less than 50%. In certain embodiments, the fraction is less than 60%. In certain embodiments, the fraction is less than 70%.

In certain embodiments, a nucleoside-modified RNA of the present disclosure is translated in the cell more efficiently than an unmodified RNA molecule with the same sequence. In certain embodiments, the nucleoside-modified RNA exhibits enhanced ability to be translated by a target cell. In certain embodiments, translation is enhanced by a factor of 2-fold relative to its unmodified counterpart. In certain embodiments, translation is enhanced by a 3-fold factor. In certain embodiments, translation is enhanced by a 5-fold factor. In certain embodiments, translation is enhanced by a 7-fold factor. In certain embodiments, translation is enhanced by a 10-fold factor. In certain embodiments, translation is enhanced by a 15-fold factor. In certain embodiments, translation is enhanced by a 20-fold factor. In certain embodiments, translation is enhanced by a 50-fold factor. In certain embodiments, translation is enhanced by a 100-fold factor. In certain embodiments, translation is enhanced by a 200-fold factor. In certain embodiments, translation is enhanced by a 500-fold factor. In certain embodiments, translation is enhanced by a 1000-fold factor. In certain embodiments, translation is enhanced by a 2000-fold factor. In certain embodiments, the factor is 10-1000-fold. In certain embodiments, the factor is 10-100-fold. In certain embodiments, the factor is 10-200-fold. In certain embodiments, the factor is 10-300-fold. In certain embodiments, the factor is 10-500-fold. In certain embodiments, the factor is 20-1000-fold. In certain embodiments, the factor is 30-1000-fold. In certain embodiments, the factor is 50-1000-fold. In certain embodiments, the factor is 100-1000-fold. In certain embodiments, the factor is 200-1000-fold. In certain embodiments, translation is enhanced by any other significant amount or range of amounts.

In certain embodiments, the nucleoside-modified antigen-encoding RNA of the present disclosure induces significantly more adaptive immune response than an unmodified in vitro-synthesized RNA molecule with the same sequence. In certain embodiments, the modified RNA molecule exhibits an adaptive immune response that is 2-fold greater than its unmodified counterpart. In certain embodiments, the adaptive immune response is increased by a 3-fold factor. In certain embodiments the adaptive immune response is increased by a 5-fold factor. In certain embodiments, the adaptive immune response is increased by a 7-fold factor. In certain embodiments, the adaptive immune response is increased by a 10-fold factor. In certain embodiments, the adaptive immune response is increased by a 15-fold factor. In certain embodiments the adaptive immune response is increased by a 20-fold factor. In certain embodiments, the adaptive immune response is increased by a 50-fold factor. In certain embodiments, the adaptive immune response is increased by a 100-fold factor. In certain embodiments, the adaptive immune response is increased by a 200-fold factor. In certain embodiments, the adaptive immune response is increased by a 500-fold factor. In certain embodiments, the adaptive immune response is increased by a 1000-fold factor. In certain embodiments, the adaptive immune response is increased by a 2000-fold factor. In certain embodiments, the adaptive immune response is increased by another fold difference.

In certain embodiments, “induces significantly more adaptive immune response” refers to a detectable increase in an adaptive immune response. In certain embodiments, the term refers to a fold increase in the adaptive immune response (e.g., 1 of the fold increases enumerated above). In certain embodiments, the term refers to an increase such that the nucleoside-modified RNA can be administered at a lower dose or frequency than an unmodified RNA molecule with the same species while still inducing an effective adaptive immune response. In certain embodiments, the increase is such that the nucleoside-modified RNA can be administered using a single dose to induce an effective adaptive immune response.

In certain embodiments, the nucleoside-modified RNA of the present disclosure exhibits significantly less innate immunogenicity than an unmodified in vitro-synthesized RNA molecule with the same sequence. In certain embodiments, the modified RNA molecule exhibits an innate immune response that is 2-fold less than its unmodified counterpart. In certain embodiments, innate immunogenicity is reduced by a 3-fold factor. In certain embodiments, innate immunogenicity is reduced by a 5-fold factor. In certain embodiments, innate immunogenicity is reduced by a 7-fold factor. In certain embodiments, innate immunogenicity is reduced by a 10-fold factor. In certain embodiments, innate immunogenicity is reduced by a 15-fold factor. In certain embodiments, innate immunogenicity is reduced by a 20-fold factor. In certain embodiments, innate immunogenicity is reduced by a 50-fold factor. In certain embodiments, innate immunogenicity is reduced by a 100-fold factor. In certain embodiments, innate immunogenicity is reduced by a 200-fold factor. In certain embodiments, innate immunogenicity is reduced by a 500-fold factor. In certain embodiments, innate immunogenicity is reduced by a 1000-fold factor. In certain embodiments, innate immunogenicity is reduced by a 2000-fold factor. In certain embodiments, innate immunogenicity is reduced by another fold difference.

In certain embodiments, “exhibits significantly less innate immunogenicity” refers to a detectable decrease in innate immunogenicity. In certain embodiments, the term refers to a fold decrease in innate immunogenicity (e.g., 1 of the fold decreases enumerated above). In certain embodiments, the term refers to a decrease such that an effective amount of the nucleoside-modified RNA can be administered without triggering a detectable innate immune response. In certain embodiments, the term refers to a decrease such that the nucleoside-modified RNA can be repeatedly administered without eliciting an innate immune response sufficient to detectably reduce production of the recombinant protein. In certain embodiments, the decrease is such that the nucleoside-modified RNA can be repeatedly administered without eliciting an innate immune response sufficient to eliminate detectable production of the recombinant protein.

In various embodiments, the composition comprises an in vitro transcribed (IVT) RNA molecule. For example, in certain embodiments, the composition of the disclosure comprises an IVT RNA molecule, which encodes an agent. In certain embodiments, the IVT RNA molecule of the present composition is a nucleoside-modified mRNA molecule.

In certain embodiments, the composition comprises at least one RNA molecule encoding a combination of at least two agents. In certain embodiments, the composition comprises a combination of two or more RNA molecules encoding a combination of two or more agents.

In certain embodiments, the present disclosure provides a method for gene editing of a cell of interest of a subject (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.). For example, the method can be used to provide one or more component of a gene editing system (e.g., a component of a CRISPR system) to a cell of interest of a subject. In some embodiments, the method comprises administering to the subject a composition comprising one or more ionizable LNP molecule formulated for targeted delivery comprising one or more nucleoside-modified RNA molecule for gene editing.

In certain embodiments, the method comprises administration of the composition to a subject. In certain embodiments, the method comprises administering a plurality of doses to the subject. In certain embodiments, the method comprises administering a single dose of the composition, where the single dose is effective in delivery of the target therapeutic agent.

In certain embodiments, the LNPs provided herein include a coding sequence for an editing enzyme encapsulated therein. Editing enzymes include various types of nucleases that are used to cut nucleic acid molecules. Such enzymes include zinc finger nucleases, Transcription activator-like effector nucleases (TALENs), meganucleases, clustered regularly interspaced short palindromic repeats (CRISPR) associated protein (CAS, e.g., CAS9), OMEGA enzymes (IscB), etc.

In certain embodiments, the nuclease is naturally occurring. In other embodiments, the nuclease is non-naturally occurring, i.e., engineered in the DNA-binding domain and/or cleavage domain. For example, the DNA-binding domain of a naturally-occurring nuclease may be altered to bind to a selected target site (e.g., CAS9 nuclease, a meganuclease that has been engineered to bind to site different than the cognate binding site). In other embodiments, the nuclease comprises heterologous DNA-binding and cleavage domains (e.g., zinc finger nucleases; TAL-effector nucleases; meganuclease DNA-binding domains with heterologous cleavage domains).

Zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms and serve as a prominent tool in the field of genome editing. In cretain embodiments, the coding sequence encodes a zinc finger.

Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). In certain embodiments, the coding sequence encodes a transcription activator-like (TAL) effector nuclease (TALEN).

In certain embodiments, the coding sequence encodes a CRISPR-associated nuclease (Cas9). “Cas9” (CRISPR associated protein 9) refers to family of RNA-guided DNA endonucleases which is characterized by two signature nuclease domains, RuvC (cleaves non-coding strand) and HNH (coding strand). Suitable bacterial sources of Cas9 include Staphylococcus aureus (SaCas9), Streptococcus pyogenes (SpCas9), and Neisseria meningitides (K M Estelt et al., Nat Meth, 10:1116-1121 (2013), incorporated herein by reference). The wild-type coding sequences may be utilized in the constructs described herein. Alternatively, the bacterial codons are optimized for expression in humans, e.g., using any of a variety of known human codon optimizing algorithms. Alternatively, these sequences may be produced synthetically, either in full or in part. Other endonucleases with similar properties may optionally be substituted. See, e.g., the public CRISPR database (db) accessible at crispr.u-psud.fr/crispr.

In certain embodiments, the coding sequence encodes a meganuclease. Meganucleases are endodeoxyribonucleases characterized by a large recognition site (double-stranded DNA sequences of 12 to 40 base pairs), for example, I-SceI. When combined with a nuclease, DNA can be cut at a specific location. The restriction enzymes can be introduced into cells, for use in gene editing or for genome editing in situ. In certain embodiments, the nuclease is a member of the LAGLIDADG (SEQ ID NO: 1) family of homing endonucleases. In certain embodiments, the nuclease is a member of the I-CreI family of homing endonucleases which recognizes and cuts a 22 base pair recognition sequence SEQ ID NO: 2—CAAAACGTCGTGAGACAGTTTG. See, e.g., WO 2009/059195. Methods for rationally-designing mono-LAGLIDADG homing endonucleases were described which are capable of comprehensively redesigning I-CreI and other homing endonucleases to target widely-divergent DNA sites, including sites in mammalian, yeast, plant, bacterial, and viral genomes (WO 2007/047859). The term “homing endonuclease” is synonymous with the term “meganuclease.” See, WO 2018/195449, describing certain PCSK9 meganucleases, which is incorporated herein in its entirety.

In certain embodiments, the compositions described herein include coding sequences for editing enzymes, particularly nucleases, which are useful targeting a gene for the insertion of a transgene. In certain situations, for example, for applications that do not require precise targeting of an existing gene or locus (e.g., to introduce or modify an endogenous gene, allele, or regulatory element), a common strategy is to target transgene integration to one of a small number of genomic “safe harbor” sites (SHS) for expression, presumably without disrupting the expression of adjacent or more distant genes. These putative SHS play an increasingly important role in developing effective gene therapies; in the investigation of gene structure, function, and regulation; and in cell-based biotechnology.

Certain SHS are known in the art, or may be discovered. Known SHS include the AAVS1 site on chromosome 19q, CCR5 chemokine receptor gene, ROSA26, PCKS9, and TTR. See, e.g., Monnat et al., New Human Chromosomal Sites with “Safe Harbor” Potential for Targeted Transgene Insertion, Hum Gene Ther. 2019 Jul. 1; 30(7): 814-828, which is incorporated by reference. In certain embodiments, the editing enzyme targets a SHS.

In certain embodiments, the editing enzyme is a nuclease that is specific for Proprotein convertase subtilisin/kexin type 9 (PCSK9). In other embodiments, the editing enzyme is a nuclease that is specific for transthyretin (TTR). See, e.g., Conway et al., Non-viral Delivery of Zinc Finger Nuclease mRNA Enables Highly Efficient In Vivo Genome Editing of Multiple Therapeutic Gene Targets, Molecular Therapy, 27(4):866-877 (April 2019), which is incorporated herein by reference. In certain embodiments, the nuclease is a meganuclease such as that described, e.g., in International Patent Publication No. WO 2018/195449.

In some embodiments, the LNP further includes sequences which direct the nuclease to a target site in the target locus. As used herein, the term “target site” or “target sequence” refers to the specific nucleotide sequence that is recognized by the editing enzyme, or its guide sequence. The “target locus” or “target gene locus” is any site in the gene coding region where insertion of the heterologous transgene is desired. For example, in certain embodiments, the target PCSK9 locus is in Exon 7 of the PCSK9 coding sequence located on chromosome 1.

In certain embodiments, such as a meganuclease specific for PCSK9 or TTR, no further sequences are required to direct the nuclease to the target site. However, in the case, for example, of Cas9, an additional sequence, called a “single guide RNA” or “sgRNA” is provided, which is specific for the target sequence. As used herein, the sgRNA has at least a 20-base sequence (or about 24-28 bases, sometimes called the seed region) for specific DNA binding (i.e., homologous to the target DNA), in combination with the gRNA scaffold. Transcription of sgRNAs should start precisely at the 5′ end. When targeting the template DNA strand, the base-pairing region of the sgRNA has the same sequence identity as the transcribed sequence. When targeting the non-template DNA strand, the base-pairing region of the sgRNA is the reverse-complement of the transcribed sequence. Optionally, the LNP may contain more than one sgRNA. The sgRNA is 5′ to a protospacer-adjacent motif (PAM) which is specifically recognized by the Cas9 (or Cpf1) enzyme. Typically, the sgRNA is “immediately” 5′ to the PAM sequence, i.e., there are no spacer or intervening sequences. Suitable sgRNAs can be designed by the person of skill in the art.

The sgRNA includes at least 20 nucleotides and specifically binds to a target site in the target gene, said target site being 5′ to a protospacer-adjacent motif (PAM) that is specifically recognized by the Cas9. The seed region in some embodiments shares 100% complementarity with the target site in the target gene. In other embodiments, the seed region contains 1, 2, 3, 4, or 5 mismatches as compared to the target site.

In other embodiments, for example, wherein the nuclease is a Cas9, the gene editing vector further includes one or more nuclear localization signal (NLSs). In certain embodiments, the NLSs flank the coding sequence for the Cas9.

In certain embodiments, the cargo is a DNA molecule or an RNA molecule. In certain embodiments, the cargo is a cDNA or mRNA molecule. In various embodiments, the composition comprises an in vitro transcribed (IVT) RNA molecule. For example, in certain embodiments, the composition comprises an IVT RNA molecule, which encodes an editing enzyme. In certain embodiments, the IVT RNA molecule is a nucleoside-modified mRNA molecule. In certain embodiments, where the nuclease coding sequence is provided as messenger RNA (mRNA). An mRNA may include a 5′ untranslated region, a 3′ untranslated region, and/or a coding or translating sequence.

An mRNA may be a naturally or non-naturally occurring mRNA. An mRNA may include one or more modified nucleobases, nucleosides, or nucleotides. In some embodiments, the mRNA in the compositions comprise at least one modification which confers increased or enhanced stability to the nucleic acid, including, for example, improved resistance to nuclease digestion in vivo. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. As used herein, the terms “modification” and “modified” as such terms relate to the nucleic acids provided herein, include at least one alteration which preferably enhances stability and renders the mRNA more stable (e.g., resistant to nuclease digestion) than the wild-type or naturally occurring version of the mRNA. As used herein, the terms “stable” and “stability” as such terms relate to the nucleic acids of the present disclosure, and particularly with respect to the mRNA, refer to increased or enhanced resistance to degradation by, for example nucleases (i.e., endonucleases or exonucleases) which are normally capable of degrading such mRNA. Increased stability can include, for example, less sensitivity to hydrolysis or other destruction by endogenous enzymes (e.g., endonucleases or exonucleases) or conditions within the target cell or tissue, thereby increasing or enhancing the residence of such mRNA in the target cell, tissue, subject and/or cytoplasm. Also contemplated by the terms “modification” and “modified” as such terms related to the mRNA of the present disclosure are alterations which improve or enhance translation of mRNA nucleic acids, including for example, the inclusion of sequences which function in the initiation of protein translation (e.g., the Kozak consensus sequence).

In some embodiments, the mRNA described herein have undergone a chemical or biological modification to render them more stable. Exemplary modifications to an mRNA include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring mRNA, for example, covalent modifications such as the introduction of modified nucleotides, (e.g., nucleotide analogs, or the inclusion of pendant groups which are not naturally found in such mRNA molecules).

In some embodiments, the number of C and/or U residues in an mRNA sequence is reduced. In certain embodiments, the number of C and/or U residues is reduced by substitution of one codon encoding a particular amino acid for another codon encoding the same or a related amino acid. Contemplated modifications to the mRNA nucleic acids of the present disclosure also include the incorporation of pseudouridines pseudouridine (ψ) or 5-methylcytosine (m5C). Substitutions and modifications to the mRNA of the present disclosure may be performed by methods readily known to one or ordinary skill in the art.

In certain embodiments, the mRNA includes a 5′ cap structure, a chain terminating nucleotide, a stem loop, a polyA sequence, and/or a polyadenylation signal. A cap structure or cap species is a compound including two nucleoside moieties joined by a linker and may be selected from a naturally occurring cap, a non-naturally occurring cap or cap analog, or an anti-reverse cap analog. An mRNA may instead or additionally include a chain terminating nucleoside.

In certain embodiments, the mRNA includes a stem loop, such as a histone stem loop. A stem loop may include 1, 2, 3, 4, 5, 6, 7, 8, or more nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

In certain embodiments, the mRNA includes a polyA sequence. A polyA sequence may be comprised entirely or mostly of adenine nucleotides or analogs or derivatives thereof. In certain embodiments, the polyA sequence is a tail located adjacent to a 3′ untranslated region of an mRNA.

In certain embodiments, the present disclosure provides a method for gene editing of a cell of interest of a subject (e.g., a bone cell and/or bone marrow cell). For example, the method can be used to provide one or more component of a gene editing system (e.g., a component of a CRISPR system) to a cell of interest of a subject. In certain embodiments, a second nucleic acid molecule is provided that encodes a transgene of interest, or an expression cassette containing the transgene coding sequence. In some embodiments, the transgene is a therapeutic agent.

Examples of suitable transgenes for delivery include, e.g., those associated with familial hypercholesterolemia (e.g., VLDLr, LDLr, ApoE, see, e.g., WO 2020/132155, WO 2018/152485, WO 2017/100682, which are incorporated herein by reference), muscular dystrophy, cystic fibrosis, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB—P51608), Amyotrophic Lateral Sclerosis (ALS), Duchenne Type Muscular dystrophy, Friedrichs Ataxia (e.g., frataxin), progranulin (PRGN) (associated with non-Alzheimer's cerebral degenerations, including, frontotemporal dementia (FTD), progressive non-fluent aphasia (PNFA) and semantic dementia), among others. Other useful gene products include, carbamoyl synthetase I, ornithine transcarbamylase (OTC), arginosuccinate synthetase, arginosuccinate lyase (ASL) for treatment of arginosuccinate lyase deficiency, arginase, fumarylacetate hydrolase, phenylalanine hydroxylase, alpha-1 antitrypsin, rhesus alpha-fetoprotein (AFP), rhesus chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)). Examples of suitable transgene for delivery may include human frataxin delivered in an AAV vector as described, e.g., PCT/US20/66167, Dec. 18, 2020, U.S. Provisional Patent Application No. 62/950,834, filed Dec. 19, 2019, and U.S. Provisional Application No. 63/136,059 filed on Jan. 11, 2021 which are incorporated herein by reference. Another example of suitable transgene for delivery may include human acid-ar-glucosidase (GAA) delivered in an AAV vector as described, e.g., PCT/US20/30493, Apr. 30, 2020, now published as WO2020/223362A1, PCT/US20/30484, Apr. 20, 2020, now published as WO 2020/223356 A1, U.S. Provisional Patent Application No. 62/840,911, filed Apr. 30, 2019, U.S. Provisional Application No. 62/913,401, filed Oct. 10, 2019, U.S. Provisional Patent Application No. 63/024,941, filed May 14, 2020, and U.S. Provisional Patent Application No. 63/109,677, filed Nov. 4, 2020 which are incorporated herein by reference. Also, another example of suitable transgene for delivery may include human α-L-iduronidase (IDUA) delivered in an AAV vector as described, e.g., PCT/US2014/025509, Mar. 13, 2014, now published as WO 2014/151341, and U.S. Provisional Patent Application No. 61/788,724, filed Mar. 15, 2013 which are incorporated herein by reference.

In certain embodiments, the transgene may be operably linked to regulatory sequences that direct the expression thereof. In some embodiments, the transgene cassette includes a promoter, the transgene coding sequence, and a poly A sequence. In other embodiments, a transgene is provided without a promoter, and is inserted in the genome downstream of a native promoter, e.g., the PCSK9 promoter.

In addition to a promoter, the transgene cassette may contain one or more appropriate “regulatory elements” or “regulatory sequences”, which comprise but are not limited to an enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., the alpha fetoprotein enhancer, the TTR minimal promoter/enhancer, LSP (TH-binding globulin promoter/alpha1-microglobulin/bikunin enhancer), amongst others. These control sequences or the regulatory sequences are operably linked to the nuclease coding sequences or transgene coding sequence.

In addition to the transgene cassette, in certain embodiments, the LNP composition described herein also includes homology-directed recombination (HDR) arms 5′ and 3′ to the transgene cassette, to facilitate homology directed recombination of the transgene into the endogenous genome. The homology arms are directed to the target locus and can be of varying length. In some embodiments, the HDR arms are from about 100 bp to about 1000 bp in length. In other embodiments, the HDR arms are from about 130 bp to about 500 bp. In other embodiments, the HDR arms are from about 100 bp to about 300 bp. In certain embodiments, the HDR arm is 130 bp. In other embodiments, the HDR arms are about 130 bp to 140 bp. In certain embodiments, the HDR arms are about 500 bp. In certain embodiments, the HDR arms are absent. The HDR arms ideally share a high level of complementarity with the target locus, although it need not be 100% complementarity. In some embodiments, 1, 2, 3, 4, 5, 6, 7, 8, 9, 10, 11, 12, 13, 14, 15, 16, 17, 18, 19, 20, or more mismatches are permitted in each HDR arm.

The ratio of ionizable lipid to nucleic acid may be varied in the LNP in a range from about 1:1 to about 10:1 by weight. In certain embodiments, the lipid:nucleic acid ratio is about 5:1. In certain embodiments, the lipid:nucleic acid ratio is about 6:1. In certain embodiments, the lipid:nucleic acid ratio is about 7:1. In certain embodiments, the lipid:nucleic acid ratio is about 8:1. In certain embodiments, the lipid:nucleic acid ratio is about 9:1. In certain embodiments, the lipid:nucleic acid ratio is about 10:1.

In embodiments where the composition includes a coding sequence for a Cas9 (e.g., mRNA encoding Cas9), the mRNA to sgRNA ratio can be present in a range of from about 1:5 to about 5:1 by weight. In certain embodiments, the mRNA:sgRNA ratio is about 1:5. In certain embodiments, the mRNA:sgRNA ratio is about 1:4. In certain embodiments, the mRNA:sgRNA ratio is about 1:3. In certain embodiments, the mRNA:sgRNA ratio is about 1:2. In certain embodiments, the mRNA:sgRNA ratio is about 1:1. In certain embodiments, the mRNA:sgRNA ratio is about 2:1. In certain embodiments, the mRNA:sgRNA ratio is about 3:1. In certain embodiments, the mRNA:sgRNA ratio is about 4:1. In certain embodiments, the mRNA:sgRNA ratio is about 5:1. Other ratios within this range can be utilized.

LNP formation and encapsulation of cargo may be accomplished using techniques known in the art. See, e.g., Jeffs, et al (March 2005). A Scalable, Extrusion-Free Method for Efficient Liposomal Encapsulation of Plasmid DNA. Pharmaceutical Research, 22(3), 362-372, and Kulkami et al., On the Formation and Morphology of Lipid Nanoparticles Containing Ionizable Cationic Lipids and siRNA, ACS Nano, 12:4787-4795 (April 2018) both of which are incorporated herein by reference. For example, in brief, LNP-mRNA compositions are generated by rapid mixing of lipids in ethanol with mRNA in aqueous buffer (pH 4.0), followed by dialysis to remove ethanol and to raise the pH to 7.4.

### Combinations

In certain embodiments, the composition of the present disclosure comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, in certain embodiments, the composition comprises a 1:1 ratio of two individual agents. However, the combination is not limited to any particular ratio. Rather any ratio that is shown to be effective is encompassed.

### Treatment Methods

The present disclosure provides methods of delivering an agent to a cell of interest of a target subject. Exemplary cells that can be targeted using the LNP compositions of the disclosure include, but are not limited to, a bone cell and/or bone marrow cell (e.g., a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.). Thus, in various embodiments, the disclosure relates to methods delivering an agent to a bone, bone marrow, or a combination thereof.

In some embodiments, the agent is a diagnostic agent to detect at least one marker associated with a disease, disorder, or condition. In some embodiments, the agent is a therapeutic agent for the treatment or prevention of a disease, disorder, or condition. In some embodiments, the agent is an editing enzyme for gene editing. In some embodiments, the agent replaces at least one protein in a subject in need thereof. Therefore, in some embodiments, the disclosure provides methods for diagnosing, treating, or preventing a disease, disorder, or condition comprising administering an effective amount of the LNP composition comprising one or more diagnostic or therapeutic agents, one or more adjuvants, or a combination thereof.

For example, in certain embodiments, the disease, disorder, or condition is a bone disease or disorder, bone marrow disease or disorder, condition associated with bone, condition associated with bone marrow, bone fracture, bone defect, root canal, nonunion fracture, cancer, leukemia, lymphoma, myeloma, other blood cancers, myeloma, sickle cell disease, inflammatory disease or disorder, or any combination thereof.

In some embodiments, the disclosure relates to methods of treating or preventing bone diseases, disorders, or conditions in subjects in need thereof, the method comprising administering the LNP composition of the disclosure. Exemplary bone diseases, disorders, or conditions that can be treated using the LNP compositions and methods of the disclosure include, but are not limited to, osteoporosis, fracture, bone defect, bone cavity, root canal, nonunion fracture, scoliosis, Paget's disease, osteoarthritis, rheumatoid arthritis, gout, bursitis, solid tumor cancer that metastasizes to bone, leukemia, lymphoma, myeloma, other blood cancers, or any combination thereof.

For example, in certain embodiments, bone diseases, disorders, or conditions that can be treated using the LNP compositions and methods of the is a fracture, bone defect, bone cavity, root canal, nonunion fracture, or any combination thereof. Thus, in some embodiments, the disclosure relates to methods of bone regeneration in subjects in need thereof, the method comprising administering the LNP composition of the disclosure.

In some embodiments, the disclosure relates to methods of treating or preventing bone marrow diseases, disorders, or conditions in subjects in need thereof, the method comprising administering the LNP composition of the disclosure. Exemplary bone marrow diseases, disorders, or conditions that can be treated using the LNP compositions and methods of the disclosure include, but are not limited to, leukemia, myelodysplastic syndrome (MDS), myeloproliferative disorders (MPD), aplastic anemia, iron deficiency anemia, disorders of plasma cells, plasma cell dyscrasia, lymphomas, thrombotic thrombocytopenic purpura, sickle cell disease, myeloma, a disease, disorder, or condition arising from hematopoietic stem cells, or any combination thereof.

In some embodiments, the disclosure relates to methods of treating or preventing cancer and diseases or disorders associated therewith in subjects in need thereof, the method comprising administering the LNP composition of the disclosure.

In some embodiments, the present disclosure provides a method for inducing an immune response in subjects in need thereof, the method comprising administering the LNP composition of the disclosure. For example, in certain embodiments, the method for inducing an immune response in subjects in need thereof is a cancer immunotherapy comprising administering the LNP comprising CAR to the subject to induce an immune response against cancer.

Exemplary cancers that can be treated using the LNP compositions and methods of the disclosure include, but are not limited to, acute lymphoblastic leukemia, acute myeloid leukemia, adrenocortical carcinoma, appendix cancer, basal cell carcinoma, bile duct cancer. bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

In various embodiments, the disease, disorder, or condition is a disease, disorder, or condition associated with at least one cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.). For example, in certain embodiments, the disease, disorder, or condition associated with at least one cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.) is a bone disease, disorder, or condition, bone marrow disease, disorder, or condition, cancer, or any combination thereof.

In certain embodiments, the method comprises administering a LNP composition of the disclosure comprising one or more nucleic acid molecules for treatment or prevention of a disease, disorder, or condition (e.g., a bone disease, disorder, or condition, bone marrow disease, disorder, or condition, cancer, or any combination thereof). In certain embodiments, the one or more nucleic acid molecules encode a therapeutic agent for the treatment of the disease, disorder, or condition (e.g., a bone disease, disorder, or condition, bone marrow disease, disorder, or condition, cancer, or any combination thereof).

In certain embodiments, the compositions of the disclosure can be administered in combination with one or more additional therapeutic agent, an adjuvant, or a combination thereof. For example, in certain embodiments, the method comprises administering an LNP composition comprising a nucleic acid molecule encoding one or more agent for targeted administration to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-bone precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.) and a second LNP comprising a nucleic acid molecule encoding one or more adjuvants. In certain embodiments, the method comprises administering a single LNP composition comprising a nucleic acid molecule encoding one or more agent for targeted administration to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.) and a nucleic acid molecule encoding one or more adjuvants.

In certain embodiments, the method comprises administering to subject a plurality of LNPs of the disclosure comprising nucleoside-modified nucleic acid molecules encoding a plurality of agents to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.), adjuvants, or a combination thereof.

In certain embodiments, the method comprises administering the LNP of the disclosure comprising nucleoside-modified RNA, which provides stable expression of a nucleic acid encoded agent (e.g., a therapeutic agent encoded by a nucleoside modified mRNA molecule) described herein to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.).

Administration of the compositions of the disclosure in a method of treatment can be achieved in a number of different ways, using methods known in the art. In certain embodiments, the method of the disclosure comprises systemic administration of the subject, including for example enteral or parenteral administration. In certain embodiments, the method comprises intradermal delivery of the composition. In certain embodiments, the method comprises intravenous delivery of the composition. In some embodiments, the method comprises intramuscular delivery of the composition. In certain embodiments, the method comprises subcutaneous delivery of the composition. In certain embodiments, the method comprises inhalation of the composition. In certain embodiments, the method comprises intranasal delivery of the composition.

It will be appreciated that the composition of the disclosure may be administered to a subject either alone, or in conjunction with another agent.

The therapeutic and prophylactic methods of the disclosure thus encompass the use of pharmaceutical compositions comprising at least one LNP of the disclosure comprising an agent (e.g., an mRNA molecule) described herein, to practice the methods of the disclosure. The pharmaceutical compositions useful for practicing the disclosure may be administered to deliver a dose of from 0.001 ng/kg/day and 100 mg/kg/day. For example, in some embodiments, the pharmaceutical compositions useful for practicing the disclosure may be administered to deliver a dose of from 0.005 mg/kg/day and 5 mg/kg/day. In certain embodiments, the disclosure envisions administration of a dose which results in a concentration of the compound of the present disclosure from 10 nM and 10 μM in a mammal.

Typically, dosages which may be administered in a method of the disclosure to a mammal, preferably a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 0.1 μg to about 10 mg per kilogram of body weight of the mammal. More preferably, the dosage will vary from about 1 μg to about 5 mg per kilogram of body weight of the mammal. For example, in some embodiments, the dosage will vary from about 0.005 mg to about 5 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of a composition of the present disclosure may be performed by single administration or boosted by multiple administrations.

In certain embodiments, the disclosure includes a method comprising administering a combination of LNP compositions described herein. In certain embodiments, the combination has an additive effect, wherein the overall effect of the administering the combination is approximately equal to the sum of the effects of administering each LNP composition. In other embodiments, the combination has a synergistic effect, wherein the overall effect of administering the combination is greater than the sum of the effects of administering each LNP composition.

In some aspects of the disclosure, the method provides for delivery of compositions for gene editing or genetic manipulation to a target cell (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.) of a subject to treat or prevent a disease, disorder, or condition (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.).

### Therapy

In one aspect, the therapeutic compounds or compositions of the disclosure may be administered prophylactically (i.e., to prevent disease, disorder, or condition, such as a bone disease, disorder, or condition, bone marrow disease, disorder, or condition, cancer, or any combination thereof) or therapeutically (i.e., to treat disease, disorder, or condition, such as a bone disease, disorder, or condition, bone marrow disease, disorder, or condition, cancer, or any combination thereof) to subjects suffering from or at risk of (or susceptible to) developing the disease, disorder, or condition (e.g., a bone disease, disorder, or condition, bone marrow disease, disorder, or condition, cancer, or any combination thereof). Such subjects may be identified using standard clinical methods.

In the context of the present disclosure, prophylactic administration occurs prior to the manifestation of overt clinical symptoms of disease, disorder, or condition (e.g., a bone disease, disorder, or condition, bone marrow disease, disorder, or condition, cancer, or any combination thereof), such that the disease, disorder, or condition is prevented or alternatively delayed in its progression. In the context of the field of medicine, the term “prevent” encompasses any activity which reduces the burden of mortality or morbidity from a disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The composition of the disclosure can be useful in combination with therapeutic, anti-cancer, and/or radiotherapeutic agents. Thus, the present disclosure provides a combination of the present LNP with therapeutic, anti-cancer, and/or radiotherapeutic agents for simultaneous, separate, or sequential administration. The composition of the disclosure and the other anticancer agent can act additively or synergistically.

The therapeutic agent, anti-cancer agent, and/or radiation therapy can be administered according to therapeutic protocols well known in the art. It will be apparent to those skilled in the art that the administration of the therapeutic agent, anti-cancer agent, and/or radiation therapy can be varied depending on the disease being treated and the known effects of the anti-cancer agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., anti-neoplastic agent or radiation) on the patient, and in view of the observed responses of the disease to the administered therapeutic agents, and observed adverse effects.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions that are useful in the methods of the disclosure may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.10% and 99.99% (w/w) active ingredient.

In addition to the active ingredient, a pharmaceutical composition of the disclosure may further comprise one or more additional pharmaceutically active agents.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the disclosure may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Remington's Pharmaceutical Sciences (1985, Genaro, ed., Mack Publishing Co., Easton, PA), which is incorporated herein by reference.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present disclosure and practice the claimed methods. The following working examples therefore, specifically point out the preferred embodiments of the present disclosure, and are not to be construed as limiting in any way the remainder of the disclosure.

### Materials and Methods

### Chemicals and Reagents for Synthesis

1,2-Epoxy-dodecan (C12, 90%, Sigma-Aldrich), 1,2-epoxytetradecan (C14, technical grade, 85%, Sigma-Aldrich), 1,2-epoxyhexadecan (C16, technical grade, 85%, Sigma-Aldrich), acryloyl chloride (97%, Sigma-Aldrich), N-hydroxysuccinimide (NHS, 98%, TCI), 6-(p-toluidinyl)naphthalene-2-sulfonic acid (TNS, Sigma-Aldrich), triethylamine (TEA, anhydrous, 99.5%, Sigma-Aldrich), diisopropylethylamine (DIPEA, Sigma-Aldrich), tris(2-aminoethyl)amine (Core 110, 97%, Alfa Aesar), tris(3-aminopropyl)amine (Core T3A, 97%, TCI), bis(3-aminopropyl)({4-[bis(3-aminopropyl)amino]butyl})amine (Core 197, Enamine), N1-(2-(4-(2-aminoethyl)piperazin-1-yl)ethyl)ethane-1,2-diamine (Core 200, Enamine), {2-[2-(2-aminoethoxy)ethoxy]ethyl}[2-(4-{2-[2-(2-aminoethoxy)ethoxy]ethyl}piperazin-1-yl)ethyl]amine (Core 488, Enamine), 3-(4-{2-[(3-amino-2-ethoxypropyl)amino]ethyl}piperazin-1-yl)-2-ethoxypropan-1-amine (Core 490, Enamine), 2-{2-[4-(2-{[2-(2-aminoethoxy)ethyl]amino}ethyl)piperazin-1-yl]ethoxy}ethan-1-amine (Core 494, Enamine), 1,2-dioleoyl-sn-glycero-3-phosphoethanolamine (DOPE, AvantiPolarLipids), cholesterol (Sigma-Aldrich) and 1,2-dimyristoyl-sn-glycero-3-phosphoethanolamine-N-[methoxy(polyethyleneglycol)-2000](C14-PEG2000, AvantiPolarLipids), hydroxyapatite (HA) nanoparticles (Sigma-Aldrich), benzoxazolium, 3-octadecyl-2-[3-(3-octadecyl-2(3H)-benzoxazolylidene)-1-propenyl]-, perchlorate (DiO, ThermoFisher), 1,1′-dioctadecyl-3,3,3′,3′-tetramethylindotricarbocyanine iodide (DiR, ThermoFisher) and alendronate sodium (Aid, Santa Cruz) were used as received. Organic solvents were purchased from Fisher Scientific. Deuterium oxide (D20, 99.8%) and chloroform-d (CDCl3) were purchased from Acros Organics.

### Nucleic Acids and Other Reagents for Biological Assays

Luciferase 1000 Assay System (Ref. E4550) and CellTiter-Glo Luminescent Cell Viability (Ref G7572) were purchased from Promega Corporation. Alanine Transaminase (ALT) Colorimetric Activity Assay Kit (Item. 700260) and Aspartate Aminotransferase (AST) Colorimetric Activity Assay Kit (Item. 701640) were purchased from Cayman Chemical. Mouse BMP2 ELISA Kit (Ref ab119582) was purchased from abcam. Anti-mouse CD11b antibody (PE-AF610), CD45R/B220 antibody (Percp), CD117 antibody (Brilliant Violet 711), CD3 antibody (PE), Ly6c antibody (AF700), CD31 antibody (APC), Ly6G antibody (Brilliant Violet 650) and CD19 antibody (Brilliant Violet 421) were purchased from Biolegend.

### Cell Culture

Dulbecco's Modified Eagle Medium (DMEM) was purchased from Gibco containing high glucose, L-glutamine, pherol red, and without sodium pyruvate and HEPES. Trypsin-EDTA (0.25%), penicillin streptomycin (P/S) were purchased from Gibco. Fetal bovine serum (FBS) was purchased from Sigma-Aldrich. HeLa cells were cultured in DMEM supplemented with 10% FBS and 1% P/S.

### Animal Studies

C57BL/6J mice were purchased from Jackson Laboratory.

### Instruments

¹H NMR spectrum were performed on a NEO 400 MHz spectrometer. LC-MS was performed on an Agilent LCMS system equipped with UV-Vis and evaporative light scattering detectors (ELSD). Flash chromatography was performed on a Teledyne Isco CombiFlash R_{f}-200i chromatography system equipped with UV-Vis and evaporative light scattering detectors (ELSD). LNPs were formulated by a Pump33DS syringe pump (Harvard Apparatus, Holliston, MA). Particle size and zeta potentials were measured by Dynamic Light Scattering (DLS) with Malvern Zetasizer Nano ZS. Particle morphology was measured by Cryo-TEM. LNP binding efficiency to the bone surface was imaged using Leica DiM8. Flow cytometry was performed using an LSRB machine. In vitro luminescent intensity, cell viability, ALT qualification, AST qualification, TNS assay and ex vivo LNPs binding to the bone surface was quantified using an Infinite M Plex plate reader (Tecan, Morrisville, NC).

### Synthesis of N-Acryloxysuccinimide (NAS)

N-Hydroxysuccinimide (NHS, 5.75 g, 50 mmol, 1.0 equiv) and triethylamine (TEA, 8.36 mL, 60 mmol, 1.2 equiv) were dissolved in anhydrous dichloromethane (DCM; 50 mL) and the mixture was cooled to 0° C. on an ice bath. Acryloyl chloride (5.43 g, 60 mmol, 1.2 equiv) was added dropwise under vigorous stirring. The reaction was then allowed to warm to room temperature overnight. The supernatant was washed with HCl (1%; 2×50 mL), brine (2×50 mL), saturated sodium bicarbonate (2×50 mL) and brine (2×50 mL). The organic layer was collected, dried over Na₂SO₄, and concentrated in vacuo. The final pure monomer was further purified by flash chromatographic (silica gel, DCM/hexane=1/1) as a white powder. ¹H NMR (400 MHz, CDCl₃), δ 6.72-6.65 (d, 1H), 6.37-6.26 (q, 1H), 6.20-6.14 (d, 1H), 2.86 (s, 4H). LC-MS (m/z): Calcd for [M+H]⁺: 170.1, Found: 170.1.

### Synthesis of Alendronate Acrylamide

Briefly, a flame-dried 50 mL round-bottom flask was charged with NAS (101.48 mg, 0.6 mmol, 1.2 equiv), alendronate sodium (135 mg, 0.5 mmol, 1.0 equiv) and diisopropylethylamine (DIPEA, 129.24 mg, 1 mmol, 2.0 equiv), deionized water and ethanol. The reaction was conducted under 40° C. overnight. Then, the solution was concentrated in vacuo and rinsed with DCM. The obtained solid was filtered off and dissolved again in water. The final product was afforded by precipitating in acetone twice, and was used without further purification. ¹H NMR (400 MHz, D₂O), δ 6.39-6.09 (d, 2H), 5.73-5.68 (q, 1H), 6.20-6.14 (d, 1H), 3.29-3.23 (m, 2H), 2.04-1.76 (m, 4H). LC-MS (m/z): Calcd for [M+H]⁺: 304.1, Found: 304.3.

### General Synthesis of Alendronate Amide-Bearing Lipid Cores

Taking synthesis of bis(2-aminoethyl)amine-1-aminoethyl-alendronate (110BP) as an example, alendronate acrylamine (100 mg, 0.307 mmol, 1 equiv) and Core 110 (224.8 mg, 1.538 mmol, 5 equiv) were added in a glass vial equipped with a stir bar dissolved in deionized water (300 μL). The reaction was processed overnight at room temperature. Then the solution was concentrated and precipitated in acetone. The resulting oil phase was collected by centrifugation, which was dissolved in water and precipitated in acetone again. The final product was afforded after centrifugation and drying, and was used without further purification. ¹H NMR (400 MHz, D₂O), δ 3.18-3.10 (m, 2H), 2.98-2.81 (m, 6H), 2.74-2.54 (m, 10H), 1.93-1.67 (m, 4H). LC-MS (m/z): Calcd for [M+H]⁺: 450.4, Found: 450.7.

Below are the ¹H NMR shifts and mass-to-charge ratio (m/z) for amide-bearing lipid cores.

T3ABP. ¹H NMR (400 MHz, D₂O), δ 3.17-3.10 (m, 2H), 2.90-2.74 (m, 6H), 2.65-2.38 (m, 10H), 1.90-1.58 (m, 10H). LC-MS (m/z): Calcd for [M+H]⁺: 492.4, Found: 492.7.

197BP. ¹H NMR (400 MHz, D₂O), δ 3.21-3.09 (m, 2H), 2.96-2.30 (m, 22H), 2.19-2.10 (m, 2H), 1.97-1.30 (m, 16H). LC-MS (m/z): Calcd for [M+H]⁺: 620.7, Found: 620.6.

200BP. ¹H NMR (400 MHz, D₂O), δ 3.18-3.11 (m, 2H), 2.99-2.69 (m, 12H), 2.65-2.36 (m, 12H), 1.94-1.67 (m, 4H). LC-MS (m/z): Calcd for [M+H]⁺: 519.5, Found: 519.7.

488BP. ¹H NMR (400 MHz, D₂O), δ 3.67-3.57 (m, 18H), 3.18-3.11 (m, 2H), 3.09-2.93 (m, 6H), 2.92-2.71 (m, 8H), 2.61-2.42 (m, 8H), 2.02-1.68 (m, 8H). LC-MS (m/z): Calcd for [M+H]⁺: 695.7, Found: 695.6.

490BP. ¹H NMR (400 MHz, D₂O), δ 3.71-3.52 (m, 6H), 3.20-3.10 (m, 2H), 3.08-2.98 (m, 2H), 2.96-2.26 (m, 24H), 2.19-2.09 (m, 2H), 1.98-1.63 (m, 4H), 1.17-1.05 (m, 6H). LC-MS (m/z): Calcd for [M+H]⁺: 635.6. Found: 635.6.

494BP. ¹H NMR (400 MHz, D₂O), δ 3.70-3.50 (m, 10H), 3.15-3.10 (m, 2H), 3.04-2.90 (m, 4H), 2.84-2.71 (m, 6H), 2.62-2.36 (m, 12H), 2.03-1.61 (m, 4H). LC-MS (m/z): Calcd for [M+H]⁺: 607.6, Found: 607.6.

### Synthesis for Alendronate-Bearing Ionizable Lipid Libraries

An alendronate-bearing ionizable lipid library (21 ionizable lipids) was prepared by Michael addition reaction between the above described seven different BP-lipids and three different epoxide tails [1,2-epoxy-dodecane (C12), 1,2-epoxytetradecane (C14), 1,2-epoxyhexadecane (C16)]. Taking synthesis of 490BP-C14 as an example, 490BP (9.85 mg, 0.015 mmol, 1 equiv) and C14 (15.29 mg, 0.072 mmol, 4.8 equiv) were added in a glass vial equipped with a stir bar dissolved in ethanol/water (1/1). The reaction was stirred at 80° C. for three days. Then the solution was concentrated and precipitated in acetone. The crude product was afforded by removing the solvents and was used to screen the library for Luc mRNA delivery without further purification. 490BP-C14. ¹H NMR (400 MHz, CD30D), δ 3.71-3.52 (m, 10H), 3.27-3.13 (m, 2H), 3.05-2.31 (m, 18H), 2.16-1.88 (m, 2H), 1.59-1.45 (m, 8H), 1.44-1.26 (m, 80H), 1.25-1.18 (m, 6H), 0.97-0.88 (m, 12H). LC-MS (m/z): Calcd for [M+H]⁺: 1485.2, Found: 1485.3.

### BP-Free LNP Library

To evaluate the effects of BPs in enhancing bone-targeting, a similar library without BPs was synthesized using a similar protocol.

Formulation of BP Lipid-Like Materials into Lipid Nanoparticles (LNPs)

All LNPs used in this study were prepared as follows. An ethanol phase containing all lipids and an aqueous phase containing mRNA (Luc mRNA, EGFP mRNA or BMP-2 mRNA) were mixed using a microfluidic device to formulate LNPs. The ethanol phase contained alendronate-bearing ionizable lipid or normal ionizable lipid, 1,2-dioleoyl-sn-glycero-3-phosphoethanolamine (DOPE), cholesterol and 1,2-dimyristoyl-sn-glycero-3-phosphoethanolamine-N-[methoxy(polyethyleneglycol)-2000](C14-PEG2000) with a fixed molar ratio of 35%, 16%, 46.5% and 2.5%, respectively. Aqueous phase was composed of mRNA dissolved in 10 mM citrate buffer. The ethanol and aqueous phases were mixed at a flow rate of 1.8 ml/min and 0.6 ml/min (3:1) using Pump33DS syringe pumps. LNPs were dialyzed in 1× PBS using a microdialysis cassette (20,000 MWCO, Thermo Fisher Scientific, Waltham, MA) for 2 h and then filtered through a 0.22 μm filter. Zetasizer Nano was used to measure the Z-average diameters, polydispersity index (PDI) and Zeta potential. mRNA concentration and encapsulation efficiency in each LNP formulation were measured using a modified Quant-iT RiboGreen (ThermoFisher) assay on a plate reader.

In Vitro Luc mRNA LNP Library Screening

In a white transparent 96-well plate, HeLa cells were seeded at a density of 5×10³ cells per well in 100 μL growth medium (DMEM, 10% FBS, 1% P/S), and were incubated at 37° C. in 5% CO₂. The medium was exchanged for fresh growth medium, and then LNPs were treated at a dose of 10 ng Luc mRNA per well. Firefly luciferase expression was measured 24 h after LNP transfection using a Luciferase Assay System (Promega) according to the manufacturer's protocol. The luminescent signal was normalized to PBS treated cells. Cell viability was measured using a CellTiter-Glo Luminescent Cell Viability Assay (Promega), in which the luminescence was normalized to PBS treated cells according to the manufacturer's protocol.

### In Vitro Bone Targeting of LNPs Via Hydroxyapatite Binding

The affinity of BP-LNPs and LNPs in the absence of BPs toward bone mineral was investigated. DiO labelled LNP formulations were incubated with hydroxyapatite nanoparticles (HA) in Eppendorf tubes and shaking for varying lengths of time (0, 30, 60, 120, 360 and 1440 min). At the end of incubation time, HA nanoparticles and the LNPs bound to them were pelleted when the Eppendorf tubes were spun at 85×g for 5 min. The binding affinity of BP-LNPs and LNPs was determined by measuring the decrease in the relative concentration (by measuring the fluorescence intensity) of targeted BP-LNPs in the supernatant compared to LNPs in the absence of BPs. Experiment was performed in triplicate.

### Ex Vivo Bone Affinity of LNPs

Specific binding of DiO labelled 490BP-C14 LNPs to bone fragment ex vivo (femur, mice) was investigated by imaging using a fluorescent microscope. DiO labelled 490-C14 LNPs and DiO fluorescent molecules were used controls. Bone fragments were incubated with the above LNP formulation (mass concentration: 0.2 mg/mL) with different DiO concentrations (0.2% and 0.4%) or DiO solution (0.2%) for 240 min, with constant shaking. After incubation, bone was washed three times with PBS, air dried in dark, and then subjected for imaging. Representative images are shown from experiments performed in triplicate.

In Vivo Luciferase mRNA LNP Delivery

All animal procedures were performed on female C57BL/6J mice aged 6-8 weeks approved by the Institutional Animal Care & Use Committee (IACUC) of University of Pennsylvania. Mice were administered a single intravenous DiR labelled Luc mRNA at a dosage of 0.5 mg/kg via tail vein injection. The luciferase expression was evaluated using an IVIS Spectrum imaging system (Caliper Life Sciences) 12 h post-injection. Mice were then injected D-luciferin (PerkinElmer) at a dose of 150 mg/kg by intraperitoneal injection (IP). After 10 min incubation under anesthesia, bioluminescence and fluorescence intensity were quantified by measuring photon flux in the region of interest where signal emanated using Living IMAGE Software provided by Caliper. Ex vivo imaging was performed on heart, liver, spleen, lung, kidney, left leg, spine and right leg after resection.

Flow Cytometry of EGFP mRNA Transfected Diverse Cell Types in Bone Marrow

Mice were administered a single intravenous DiR labelled EGFP mRNA 490BP-C14 LNPs at a dosage of 0.5 mg/kg via tail vein injection. After 12 h, mice were euthanized by cervical dislocation and their femur and tibia were resected then collected in 1× PBS. Bone marrow was collected by flushing femurs and tibia in DMEM medium (10 mL), the obtained suspension was then centrifuged (5 min, 0.5×g) and lysed by ACK lysis buffer (ThermoFisher) (1 mL) for 10 min. Afterwards, single-cell suspensions were obtained by centrifugation (5 min, 0.5×g) and resuspended in 1× PBS (200 μL). The following fluorochrome antibodies specific to mouse were used: CD11b (PE-AF610), CD45R/B220 (Percp), CD117 (Brilliant Violet 711), CD3 (PE), Ly6c (AF700), CD31 (APC), Ly6G (Brilliant Violet 650) and CD19 (Brilliant Violet 421). The obtained single-cell suspensions were stained at 4° C. for 1 h by each of the above antibodies (3 μL), and afterwards were centrifuged, washed, centrifuged and resuspended in 1× PBS (1 mL). To define transfection efficiency of 490BP-C14 LNPs, cells from 490-C14 LNPs in the absence of BPs and PBS treated mice were used as control. Data was acquired using LSRB flow machine and analyzed with FlowJo software.

mRNA LNP-Mediated Bone Morphogenetic Protein-2 (BMP-2) Secretion in Bone Marrow

Mice were administered a single intravenous injection of 490BP-C14 LNPs encapsulating mRNA encoding for BMP-2 at dosages ranging from 0.5-1.5 mg/kg via tail vein injection. After 12 h, mice were sacrificed and their legs and spine were collected. Bone marrow was flushed with 1 mL DMEM media solution. BMP-2 was extracted from the bone surface after incubating with 4M Guanidine hydrochloride solution overnight. BMP-2 levels on the bone surface and in the bone marrow were measured using individual assay kit according to manufacturer's protocols.

### In Vivo LNP Toxicity Measurements

To evaluate the in vivo toxicity of 490BP-C14 and 490-C14 LNPs, mouse blood was drawn and serum was isolated at 12 h after injection. Alanine aminotransferase (ALT) and aspartate aminotransferase (AST) levels were measured using individual assay kits according to manufacturer's protocols.

### Example 1: Bisphosphonate Lipid-Like Materials for mRNA Delivery to Bone Microenvironment

One class of therapeutics that were of particular interest for delivery to bone were nucleic acid therapeutics, which regulated therapeutic gene expression within targeted sites to treat diseases by delivering exogenous nucleic acids, such as plasmid DNA (pDNA), small interfering RNA (siRNA), messenger RNA (mRNA), and micro RNA (miRNA). Currently, the most clinically advanced non-viral delivery vectors for RNA therapeutics were lipid nanoparticles (LNPs), a four-component formulation consisting of ionizable lipids, cholesterol, phospholipids, and polyethylene glycol (PEG)-conjugated lipids, which were used in Alnylam Pharmaceutical's siRNA therapeutic ONPATTRO and Pfizer/BioNTech's and Moderna's COVID-19 mRNA vaccines. These nanocarriers were biocompatible, reproducibly manufactured, encapsulated a range of nucleic acids, and had physical characteristics suitable for various administration routes. Recently, LNPs encapsulating siRNA were systemically delivered to bone marrow cells to silence genes in the inflammatory monocyte subset and endothelial cells to selectively inhibit migration of these cells and their progeny. Despite this encouraging progress, passive diffusion of LNPs still makes them extremely challenging to specifically deliver mRNA to the bone microenvironment and there was a critical unmet need to develop a targeted LNP technology for mRNA delivery to bone.

Although previous studies have also demonstrated that ligand substitution is a promising approach for targeted mRNA-LNPs therapeutic delivery, for instance, Peer and co-workers demonstrated that primary lymphocytes re robustly silenced by attaching a pan leukocyte selective targeting ligand onto normal lipid nanoparticles formulations (Ramishetti, S., et al., 2020, Advanced Materials, 32(12):21906128), there remained a critical unmet need to develop a targeted LNP technology for mRNA delivery to bone.

Therefore, approaches incorporating ligands with affinity for bone enhance LNP targeting and accumulation in the bone microenvironment were investigated. Among various targeting ligands, bisphosphonate (BP), such as alendronate, are analogs of inorganic pyrophosphate and chelate strongly with the calcium ion (Ca²⁺) of hydroxyapatite (HA), which is the main inorganic component of bone, endowing bisphosphonate with strong affinity and rapid adsorption to the bone surface. After administration of BP-LNPs, strong interactions between the bone-targeting ligand on the LNP surface and HA improve retention and accumulation of LNPs in the bone microenvironment.

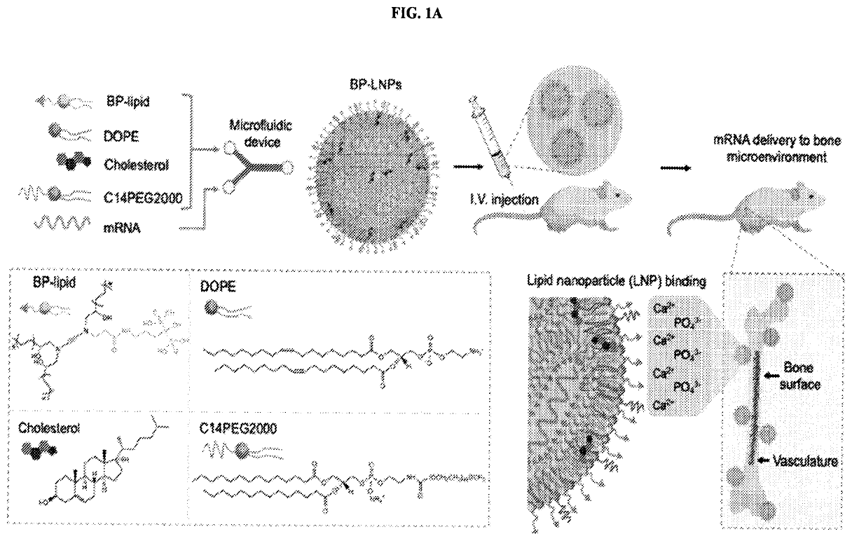

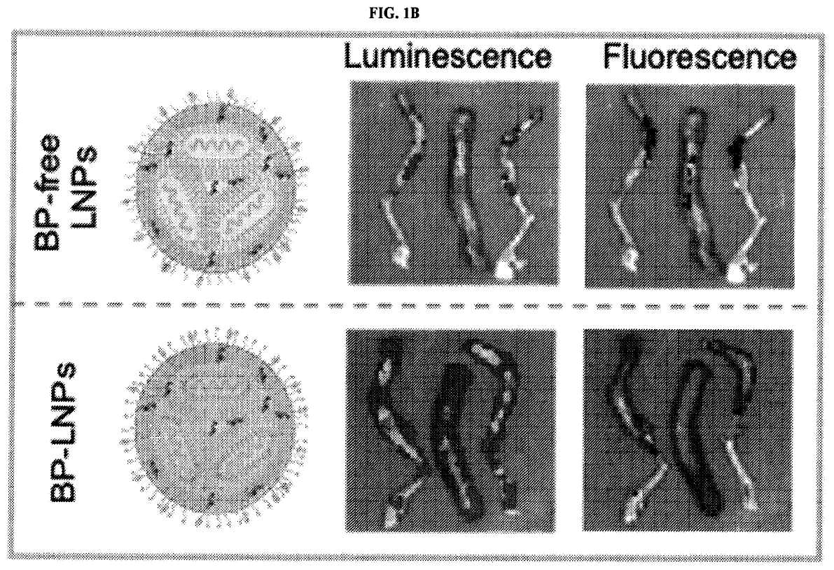

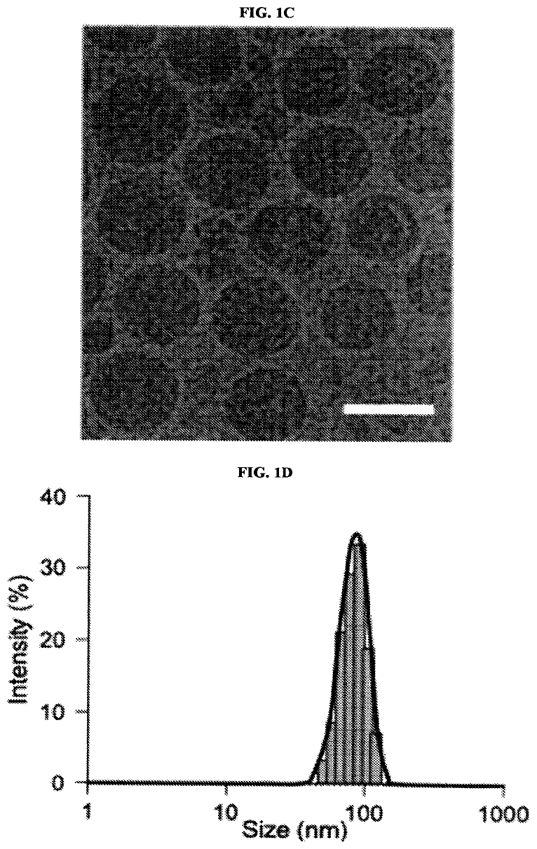

The present example describes the rational design and synthesis of a series of BP-conjugated ionizable lipid-like materials and the formulated library of BP-LNPs for enhanced systemic delivery of mRNA to the bone microenvironment (FIGS. 1A-1B). A library of twenty-one BP-functionalized ionizable lipids were investigated to formulate a library of BP-LNPs with a narrow size distribution (FIGS. 1Cs1D). Following an initial in vitro HeLa cell screening with firefly luciferase-encoding mRNA, 490BP-C14 LNP was identified as the lead formulation, which showed higher protein expression (2.5-fold) compared to LNPs in the absence of BPs (490-C14 LNP). These BP-functionalized LNPs not only displayed significantly higher affinity to both HA and bone surfaces in vitro and ex vivo, but also showed enhanced accumulation and transfection efficiency in the bone microenvironment following systemic administration in vivo (FIG. 1B). Additionally, 490BP-C14 LNPs exhibited enhanced transfection and uptake in a range of cell types in bone marrow, ranging from B cells, T cells, monocytes, granulocytes, to endothelial cells. Furthermore, BP-LNPs mediated the secretion of bone morphogenetic protein-2 (BMP-2) in the bone microenvironment (both on the bone surface and in the bone marrow) following delivery of BMP-2 mRNA via intravenous injection, demonstrating utility in therapeutic applications in bone regeneration and fracture healing. Due to the facile and versatile preparation of BP-lipid like materials, additional studies focus on applying these materials towards a range of mRNA LNP therapeutic applications in bone, including regenerative medicine, protein replacement, and gene editing therapies.

### Example 2: Rational Design and Synthesis of BP-Functionalized Ionizable Lipids and Incorporation into LNP Formulations

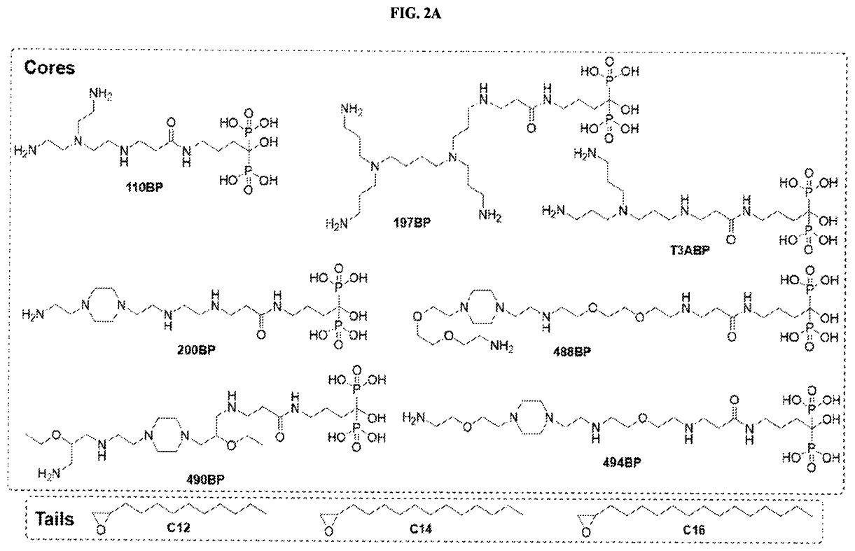

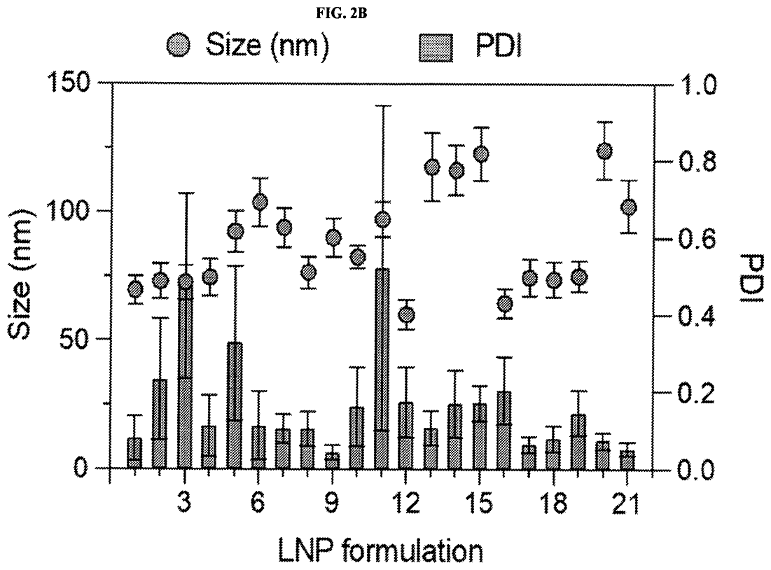

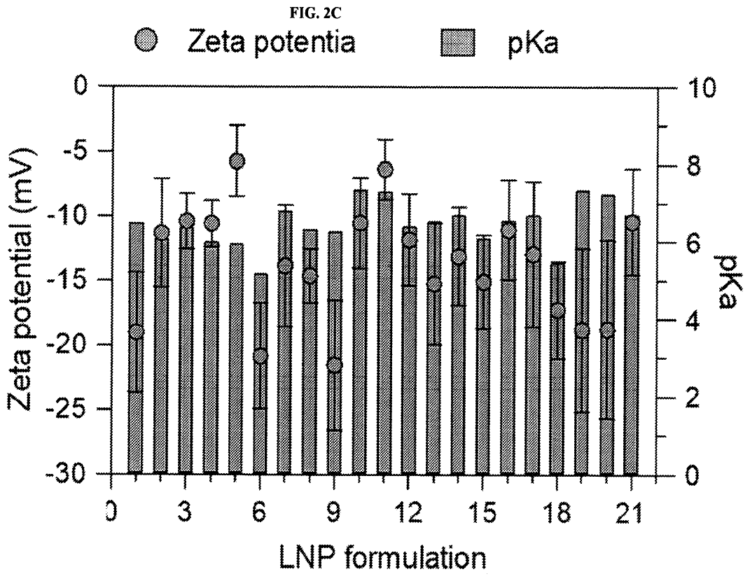

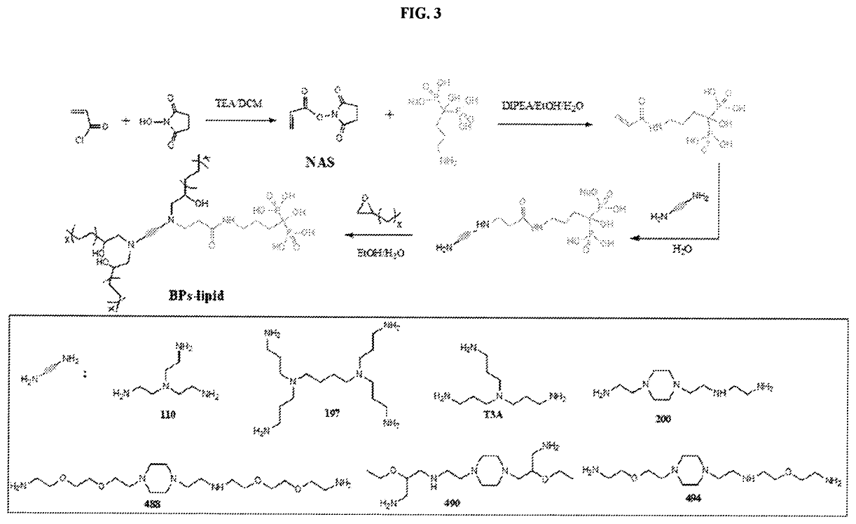

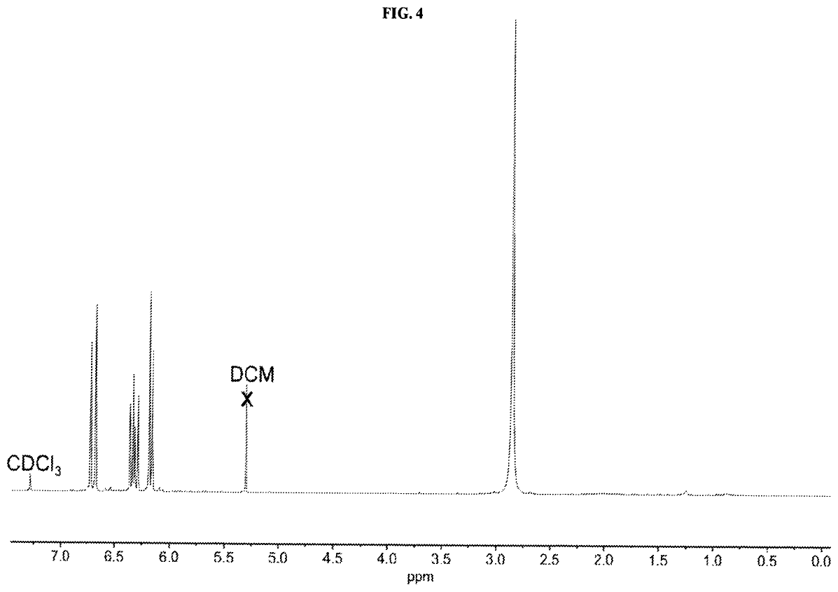

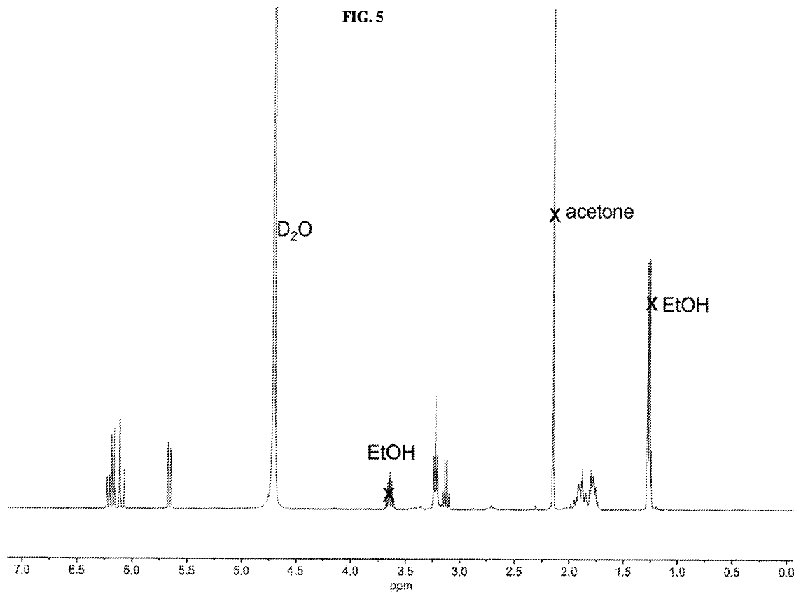

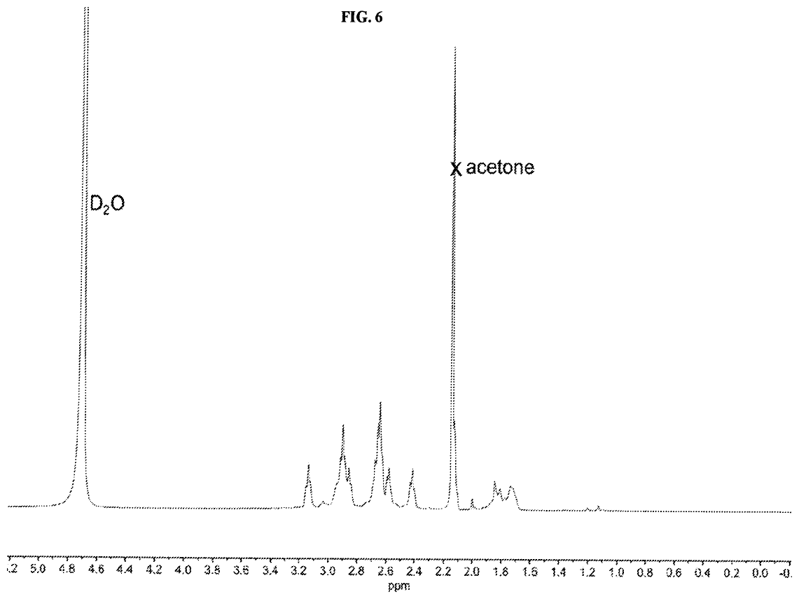

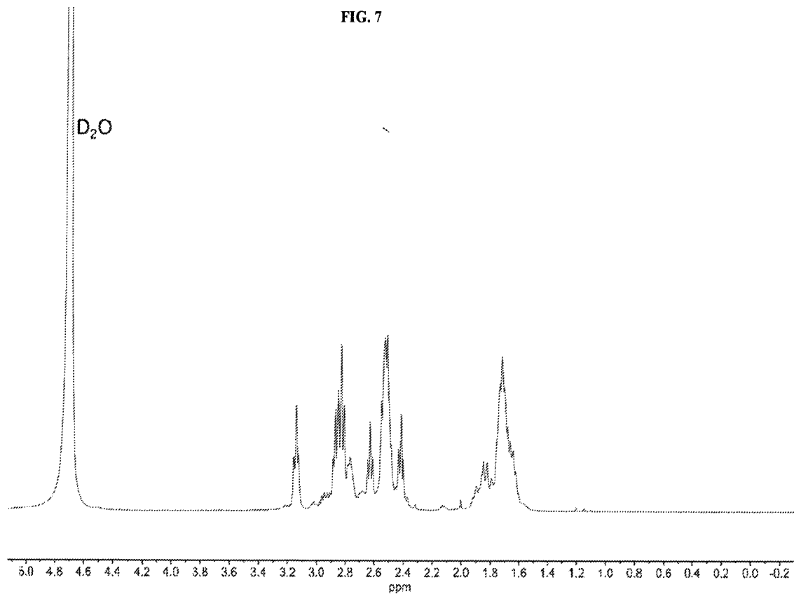

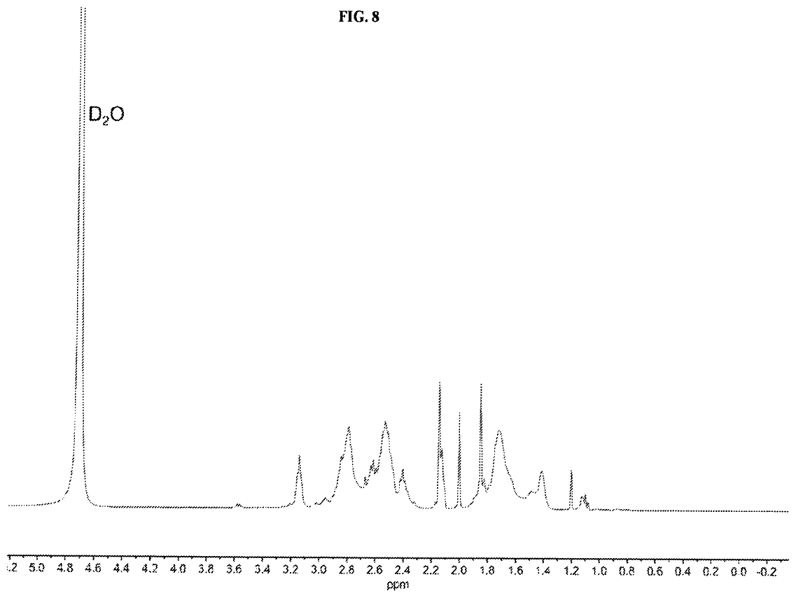

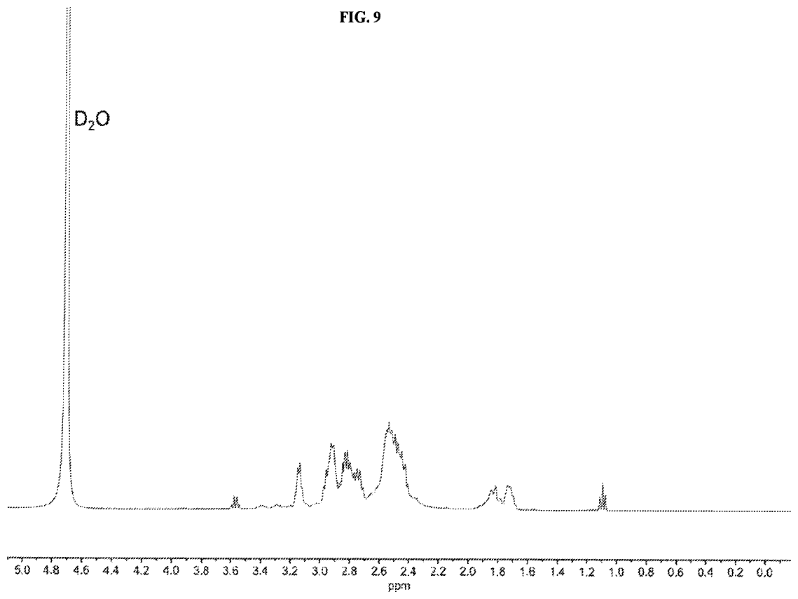

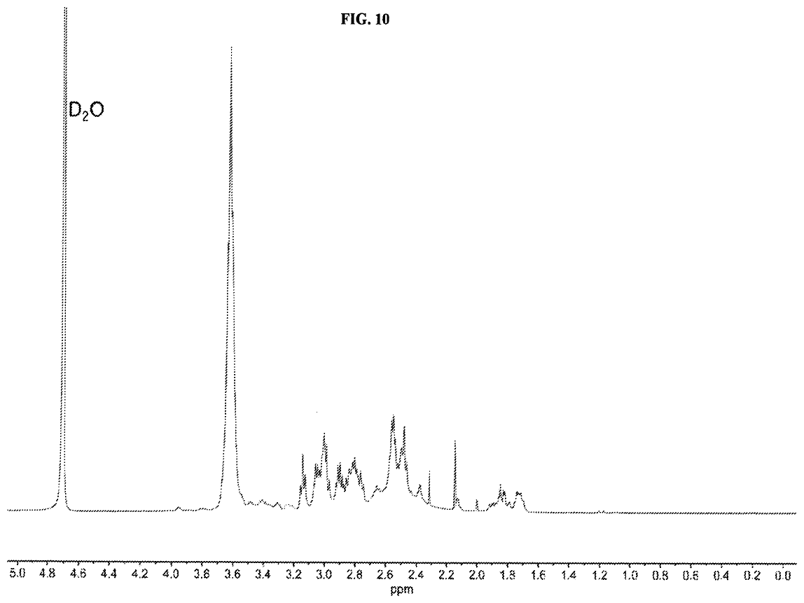

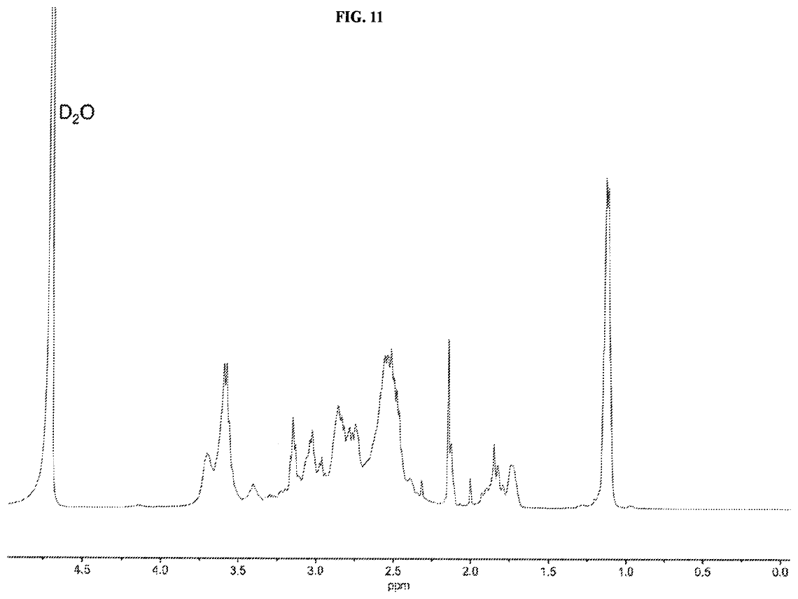

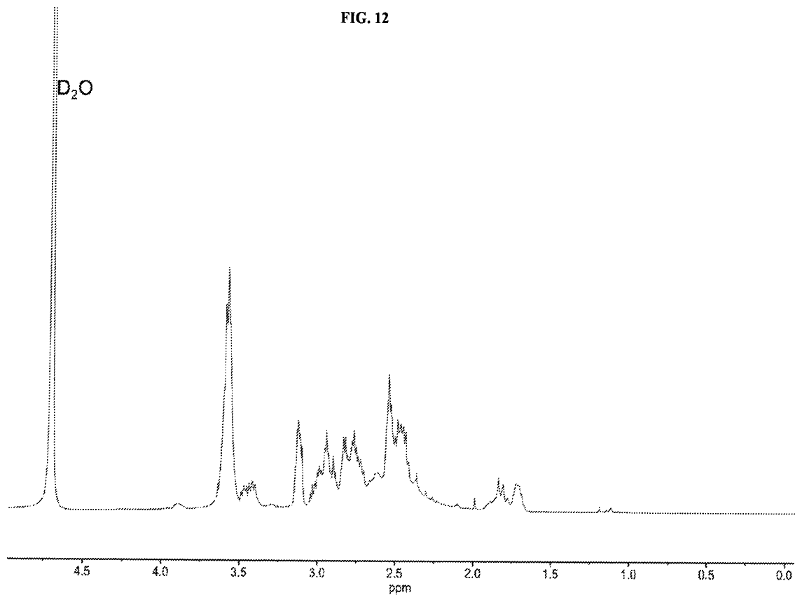

To enable bone microenvironment targeting of LNP formulations, a series of BP lipid-like materials consisting of alendronate, a type of BP, were designed and synthesized (FIG. 2A and FIG. 3). First, alendronate acrylamide was synthesized by reacting alendronate sodium with N-acryloxysuccinimide (FIGS. 3-5). The sodium salt was removed under acidic environments. Then, alendronate acrylamide was conjugated onto amine cores through a Michael addition reaction with a feeding molar ratio of 1:5, yielding alendronate-bearing amine cores. These structures were characterized by ¹H NMR spectrum and LC-MS analysis (FIGS. 5-12), demonstrating that although each amine of the core structures had the potential to conjugate alendronate, there was only one alendronate molecule featured on each core structure (FIG. 2A).

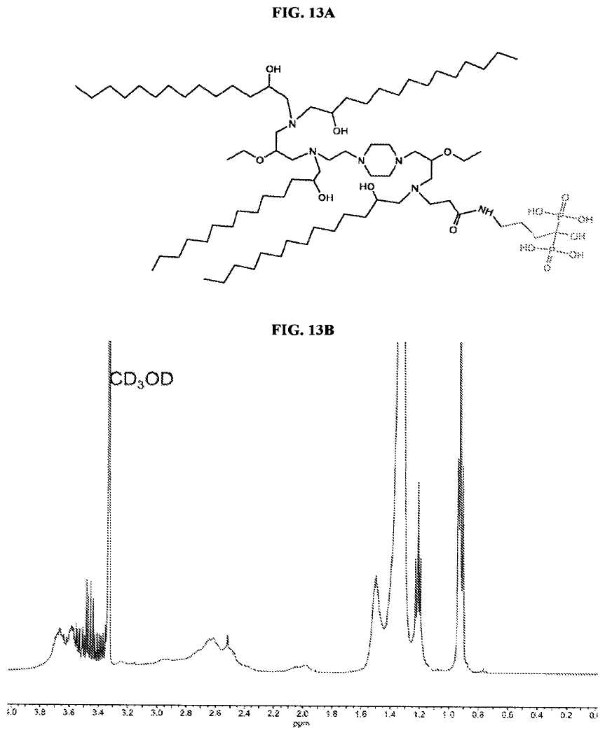

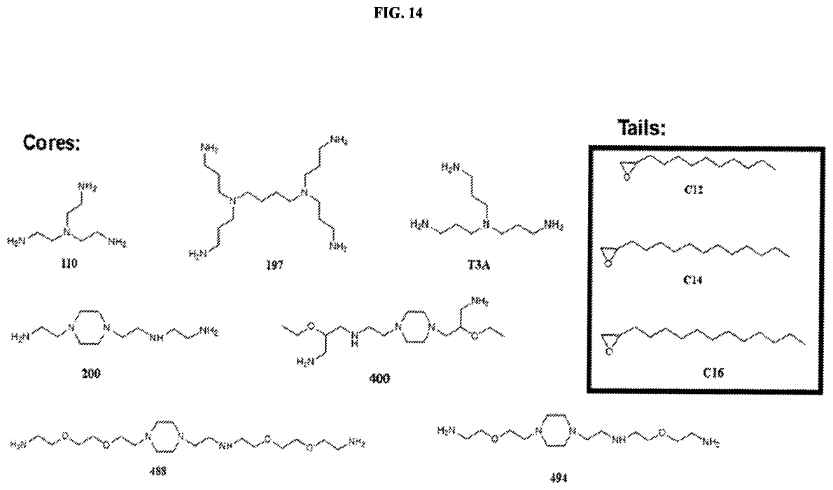

Following synthesis of alendronate-bearing amine cores, a library of twenty-one BP-lipids were synthesized by reacting the alendronate-bearing amine cores with epoxide-terminated alkyl chains (FIG. 2A and FIGS. 13A-13B). These BP-lipids were then mixed with the phospholipid DOPE, cholesterol, lipid-anchored polyethylene glycol (CT4PEG2000), and mRNA via perfusion through a microfluidic mixing device that was designed with herringbone features to induce chaotic mixing. The naming of LNP formulations represented both the unique alendronate-conjugated amine core component (labeled as 110BP, 197BP, T3ABP, 200BP, 488BP, 490BP and 494BP) and the alkyl chain length (C12, C14 and C16) (FIG. 2A). In these LNPs, the ionizable lipid enabled cellular uptake and endosomal escape to deliver encapsulated mRNA into the cytosol. DOPE and cholesterol enhanced LNP stability, improved mRNA encapsulation, and assisted in endosomal escape. To demonstrate increased delivery to bone after the introduction of alendronate molecules, another LNP library without BPs modification was formulated and used as control (FIGS. 14A-14B).

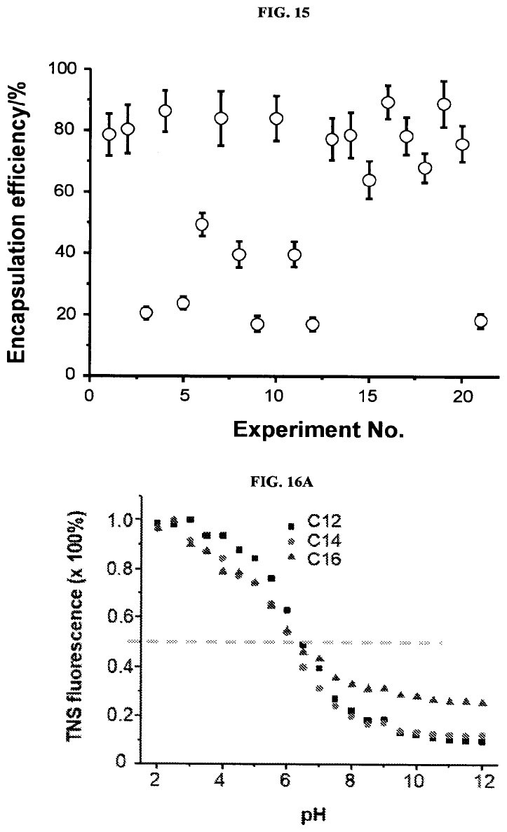

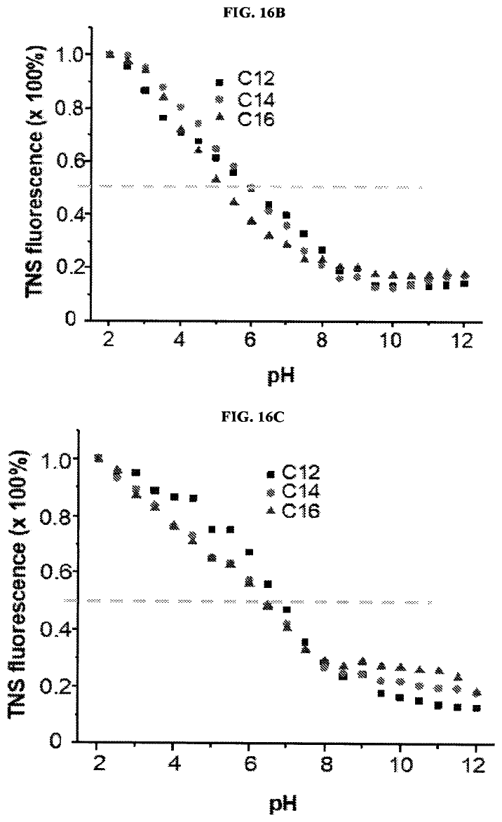

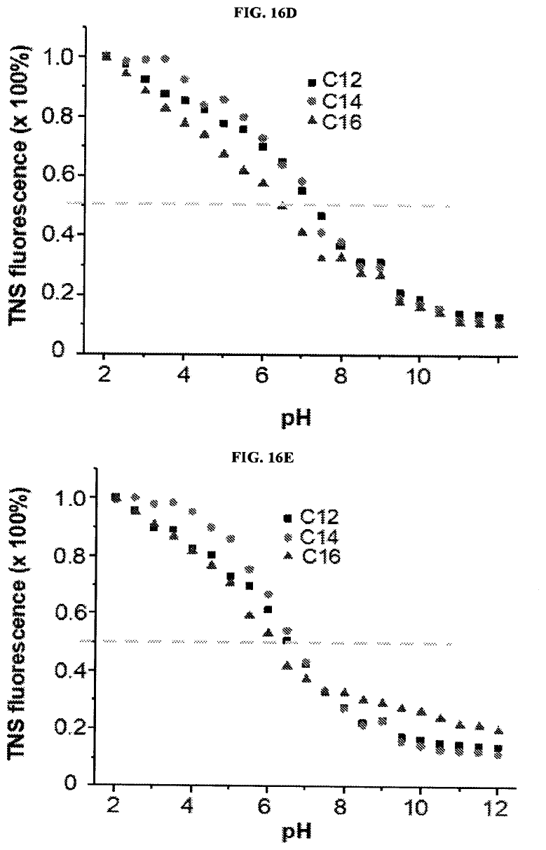

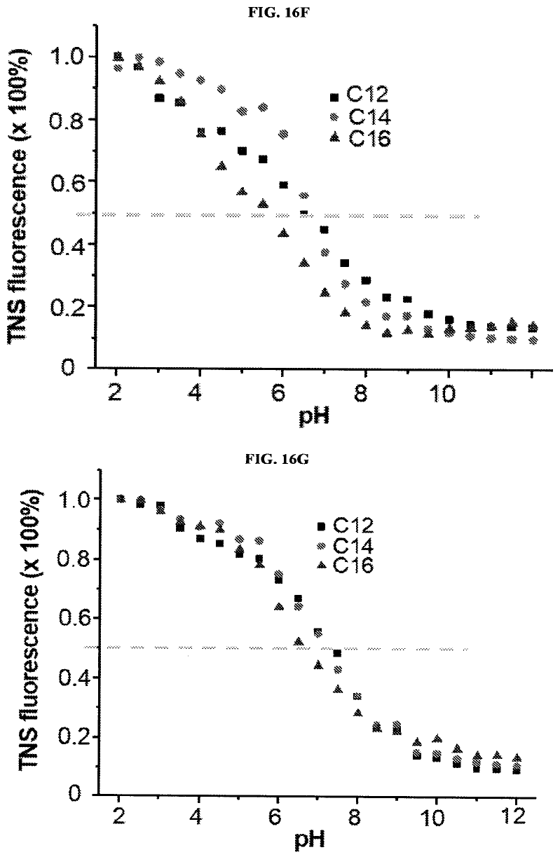

The resulting BP-LNPs were characterized to evaluate particle size, polydispersity index (PDI), zeta potential, pKa, and mRNA encapsulation efficiency. The hydrodynamic diameter for all BP-LNP formulations ranged from 60.1 to 124.2 nm by intensity measurements using dynamic light scattering (DLS) (FIG. 2B). Most BP-LNP formulations (>80%) were highly monodisperse, with a PDI value less than 0.2 (FIG. 2B). Owing to the incorporation of negatively charged alendronate molecules, BP-LNPs had a negative zeta potential (FIG. 2C). The mRNA encapsulation efficiency of BP-LNPs was assessed by a fluorescent RiboGreen assay, where most efficiencies ranged from 60 to 90%, while some LNPs containing BP lipid-like materials with a C16 alkyl chain did not formulate stable LNPs for mRNA loading due to their weak solubility during formulation (FIG. 15). Additionally, BP-LNPs were evaluated for their pKa, which indicated the pH at which BP-LNPs were 50% protonated (FIG. 2C and FIGS. 16A-16G). In previous studies, pKa values between 6 and 7 were most commonly reported for successful nucleic acid delivery as LNPs can become protonated in acidic endosomes within this range and can induce fusion with endosomal membranes to release mRNA into the cytosol. pKa values of BP-LNP formulations ranged from 5.18 to 7.35 (FIG. 2C), demonstrating that >70% of the LNPs were within the desired range (pKa 6.0-7.0) for endosomal escape and mRNA delivery.

### Example 3: BP-LNP In Vitro Screening, In Vitro Binding with Hydroxyapatite and Ex Vivo Affinity to Bone

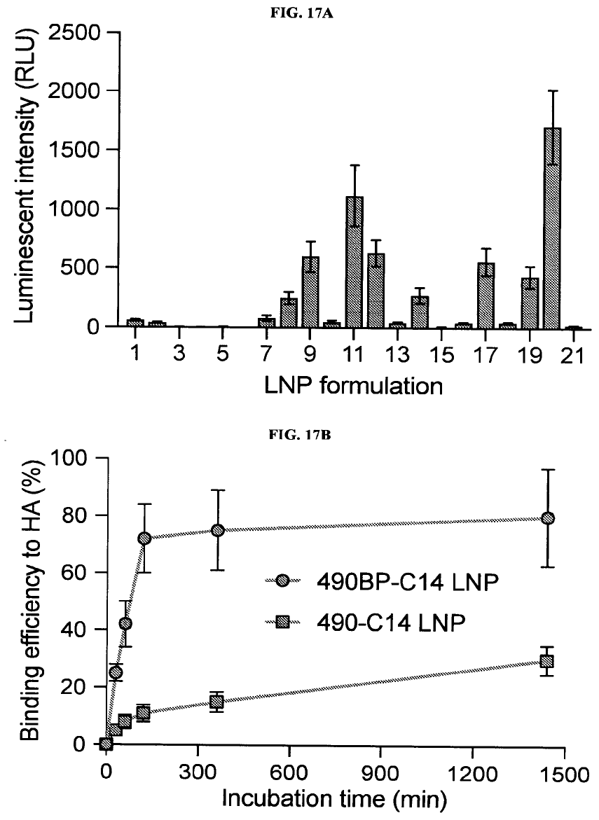

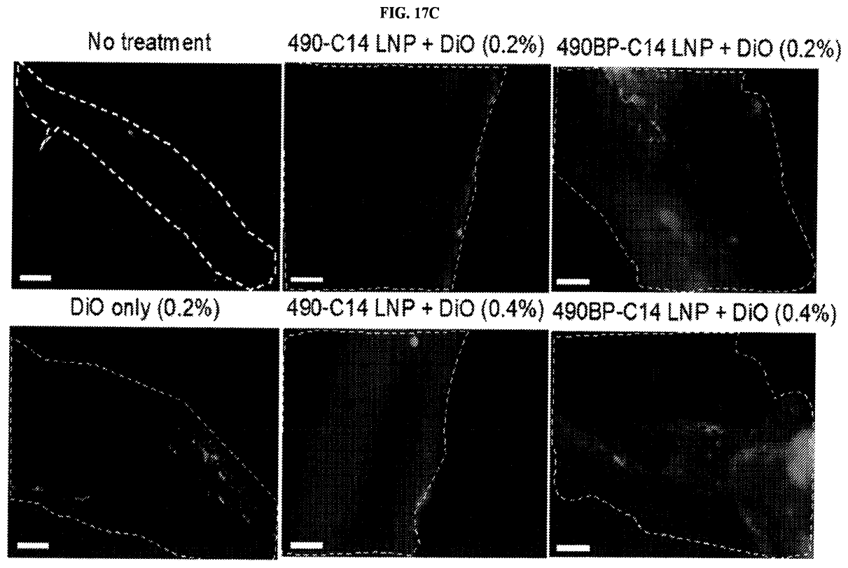

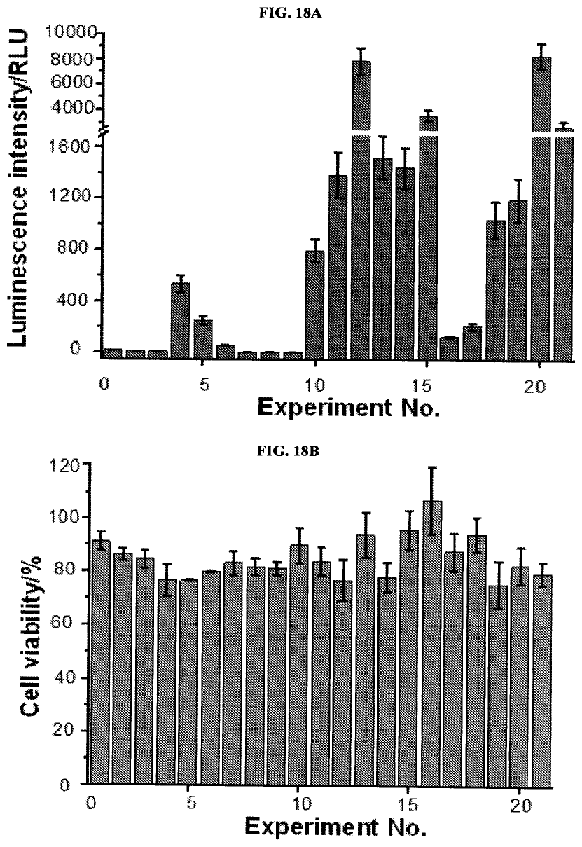

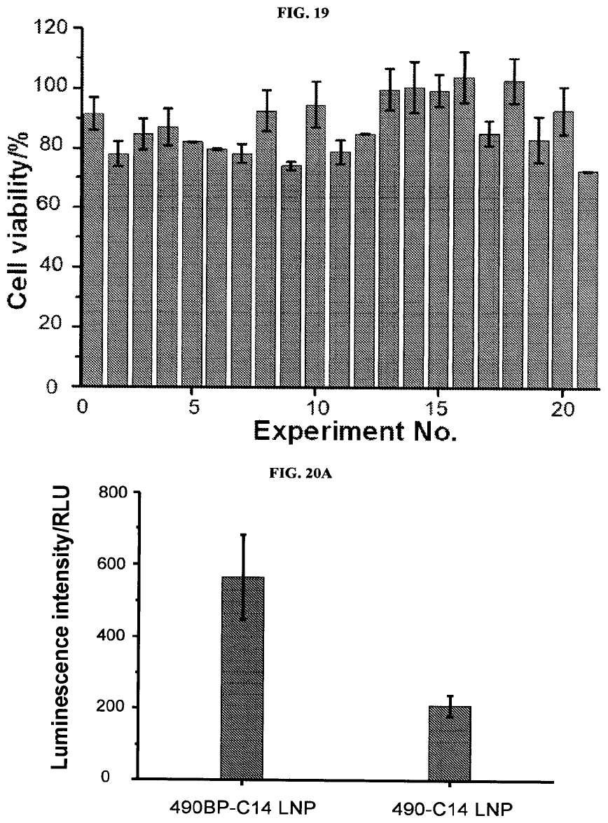

To evaluate BP-LNPs for their ability to deliver mRNA, mRNA encoding for luciferase was chosen as a reporter gene. Firefly luciferase (FLuc) mRNA with N1-Methyl-PseudoU modifications were used for screening in vitro and in vivo. These modifications have been shown to improve mRNA encapsulation efficiency, reduce overall immunogenicity, and enhance delivery of mRNA. In the present example, BP-LNP-mediated delivery of FLuc mRNA was assessed in HeLa cells, a cell line utilized for rapid in vitro screening of LNP formulations, for FLuc expression and cell viability. After 12 h treatment by BP-LNPs at a concentration of 10 ng/5000 cells, luciferase expression was evaluated via luminescence measurements, which were normalized to an untreated control group. These results allowed for determination of structure-activity relationships (SARs) with respect to epoxide-terminated alkyl tails and alendronate-amine core chemical structures, where five BP-LNPs resulted in similar or even higher luciferase expression compared to a standard LNP formulation using C12-200 as the ionizable lipid (FIG. 17A and FIGS. 18A-18B). All LNP formulations demonstrated low cytotoxicity; three formulations had cell viability between 70%-80%, while the remaining LNP formulations were >80% (FIG. 19).

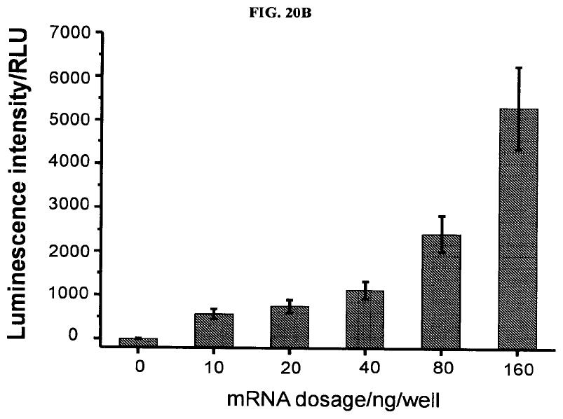

To further evaluate how the BP affects the ability of LNPs to deliver mRNA, a series of LNPs that did not incorporate BPs were formulated as controls (FIGS. 14A-14B and FIGS. 18A-18B). Among all of the LNP formulations examined in the initial screening, 490BP-C14 LNP was identified as the top formulation, as it demonstrated nearly 3 times higher transfection compared to 490-C14 LNPs in the absence of BPs (FIG. 20A). 490BP-C14 LNPs also demonstrated mRNA dose-dependent transfection of HeLa cells, showing increased luminescence intensity with increasing FLuc mRNA dosage (FIG. 20B). Therefore, 490BP-C14 was selected as the lead LNP formulation to further evaluate its ability to target bone for enhanced delivery of mRNA in vitro and in vivo.

To better explore and visualize the bone-binding efficiency of these BP-LNPs, a DiO fluorescence molecule was incorporated into both BP-LNPs and LNPs in the absence of BPs.

As the bone microenvironment is rich in HA, first an HA binding assay of 490BP-LNPs in comparison to 490-C14 LNPs that did not contain BPs was performed (FIG. 17B). After incubation with HA nanoparticles, 490BP-C14 LNPs showed fast and efficient binding to HA (FIG. 17B), owing to the strong chelation of BPs on the LNP surface with HA. After a 2 h incubation, HA binding of BP-LNPs increased to 80%, significantly higher than LNPs without BPs (10%). After this time course, the binding efficiency plateaued with increased incubation time. The affinity of BP-LNPs to HA in bone fragments was further confirmed by fluorescence microscopy (FIG. 17C). Minimal fluorescence was detected on DiO-treated bone samples, indicating low binding between free DiO molecules and the bone surface. After treatment with DiO labelled 490-C14 LNPs, very weak fluorescence signal was generated, which resulted from passive accumulation of particles on the bone surface. This weak fluorescence signal was improved by increasing the DiO concentration on 490-C14 LNPs. However, 490BP-C14 LNPs exhibited the strongest binding affinity to bone fragments, indicating that the incorporation of BPs enabled LNP targeting to bone. These results demonstrated the impact of the design of targeted BP-LNPs in bone mineral binding, and the differential binding of targeted BP-LNPs that facilitated the following in vivo studies.

### Example 4: In Vivo Transfection and Biodistribution Studies of BP-LNPs

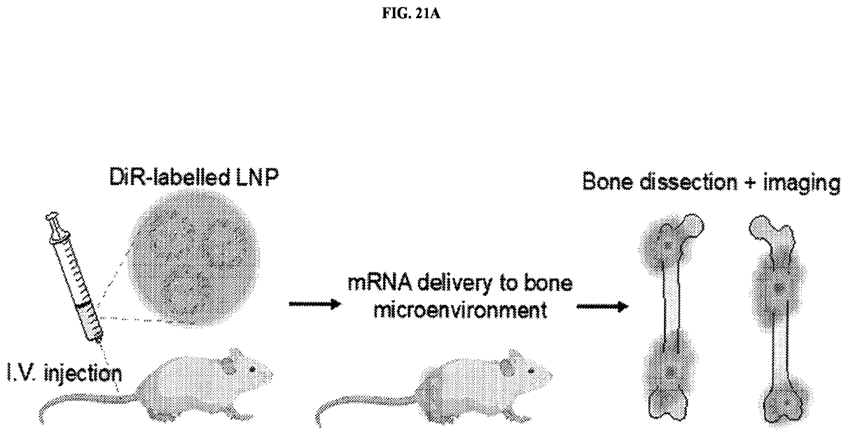

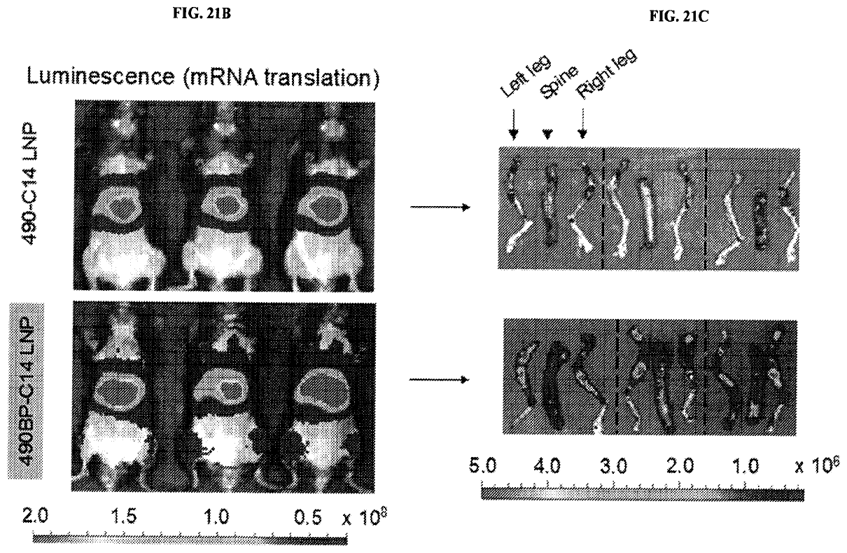

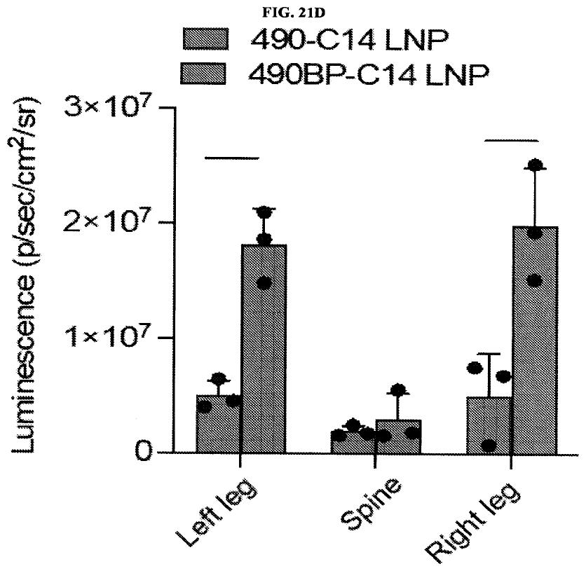

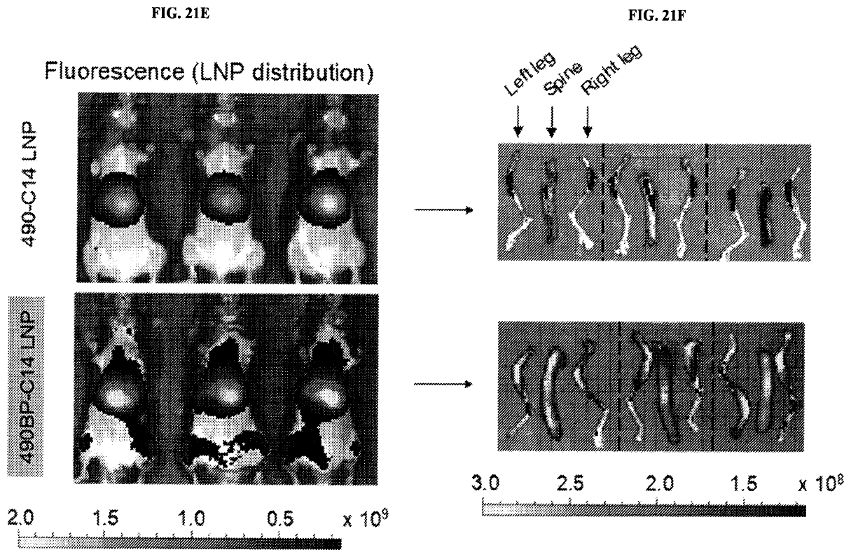

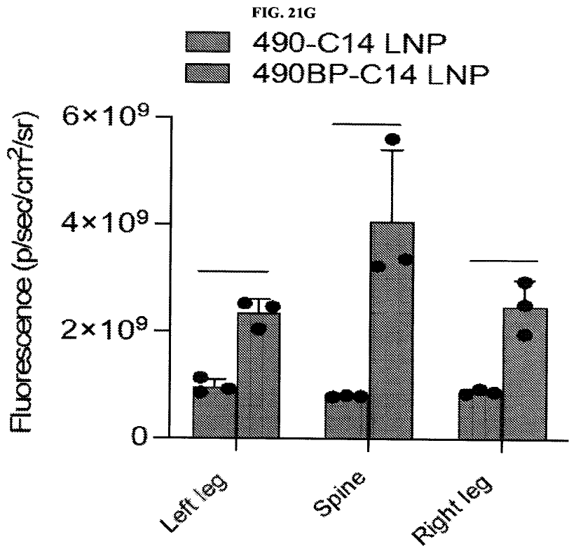

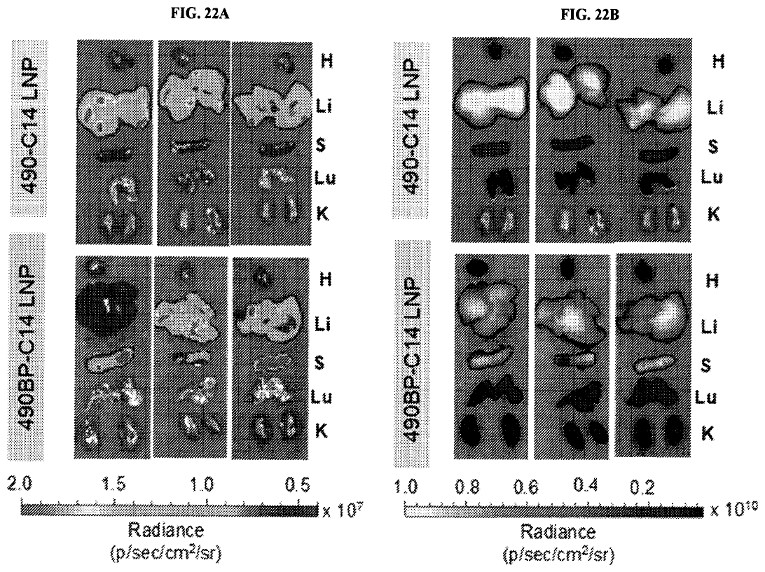

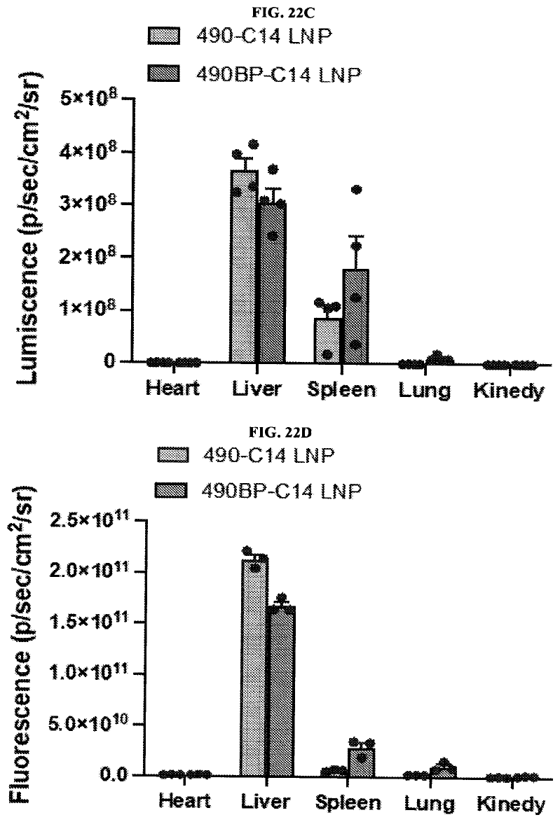

Following confirmation of the in vitro and ex vivo bone-targeting ability of BP-conjugated LNPs, in vivo transfection and biodistribution studies to investigate the bone-homing ability of BP-LNPs were performed using C57BL/6J mice following intravenous injection of 0.5 mg/kg FLuc mRNA. Fluorescent DiR was used to track LNP biodistribution using an in vivo imaging system (IVIS) (FIG. 21A). After 12 h, functional delivery of FLuc mRNA was observed in the liver and bones (FIGS. 21B-21D and FIG. 22). The luminescent intensity of the hind limbs was significantly higher following delivery of BP-LNPs (FIG. 21B). Bones from their hind limbs and spines of mice were then dissected to evaluate luminescence intensity by quantifying radiance efficiency (FIG. 21C), where LNPs without BPs showed very low transfection in the dissected bones, while BP-LNPs transfected cells on these bone surfaces, especially in the hind limbs, such as the femur and tibia (FIG. 21D). The mean transfection efficiency of left leg, spine, and right leg increased 3.7, 1.8, and 3.7 times, respectively, with BP-LNPs compared to LNPs without BPs.

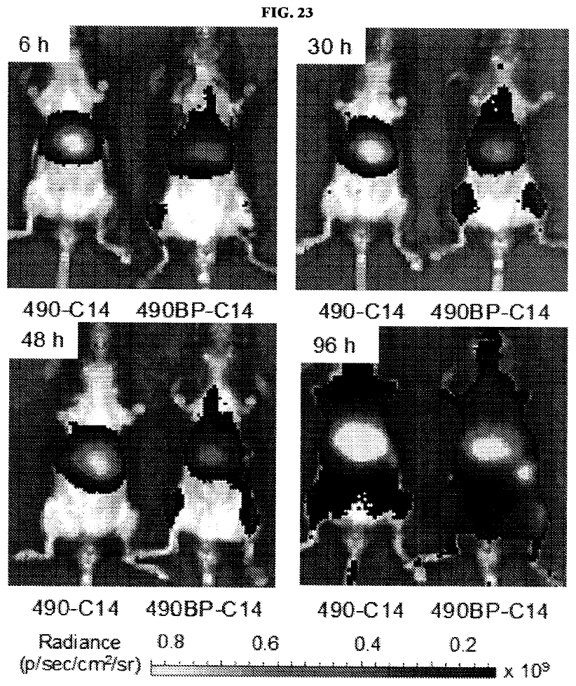

The biodistribution of BP-LNPs was then investigated (FIGS. 21E-21G). While LNPs without BPs mainly distributed in the liver, BP-LNP biodistribution was more widespread in liver/spleen and bone-dense regions (FIG. 21E). By observing the biodistribution of BP-LNPs over time, the fluorescence intensity of the whole body of mice increased, implying the adsorption of BP-LNPs during circulation (FIG. 23). For further examination of bone-targeting, dissected legs and spine were evaluated by quantifying the fluorescent intensity (FIG. 21F). All BP-LNP treated bone samples exhibited significantly increased fluorescence (FIG. 21G), with approximately 2.5, 4.5, 2.4-fold increases in the left leg, spine and right leg, respectively, compared to LNPs without BPs. Thus, in vivo transfection and biodistribution studies complement the n vitro and ex vivo high bone binding efficiency of BP-LNPs, highlighting their utility for bone-targeted mRNA delivery.

### Example 5: In Vivo BP-LNP Transfection and Distribution of Diverse Cell Types in Bone and Bone Marrow

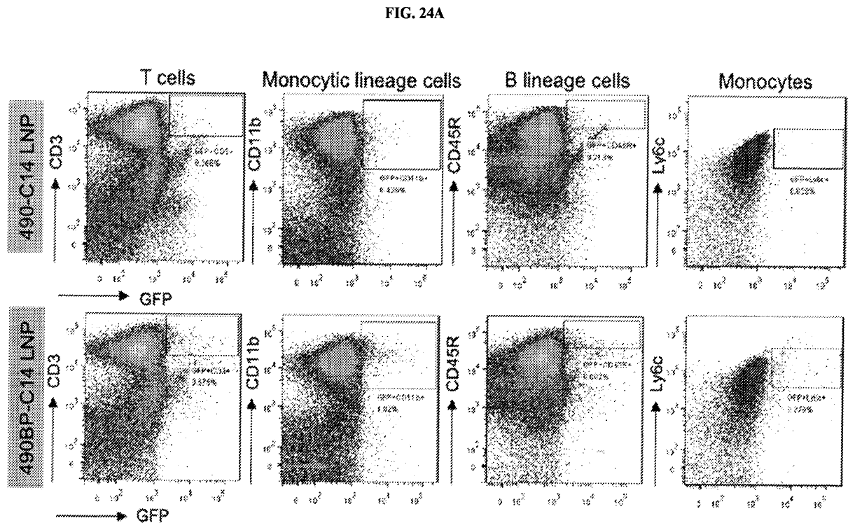

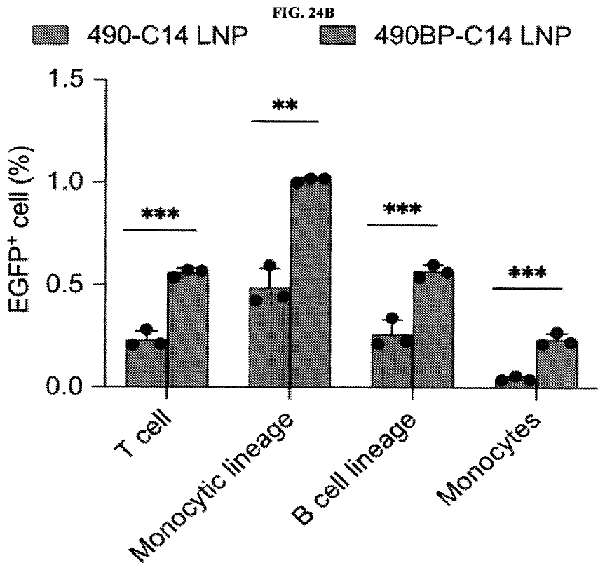

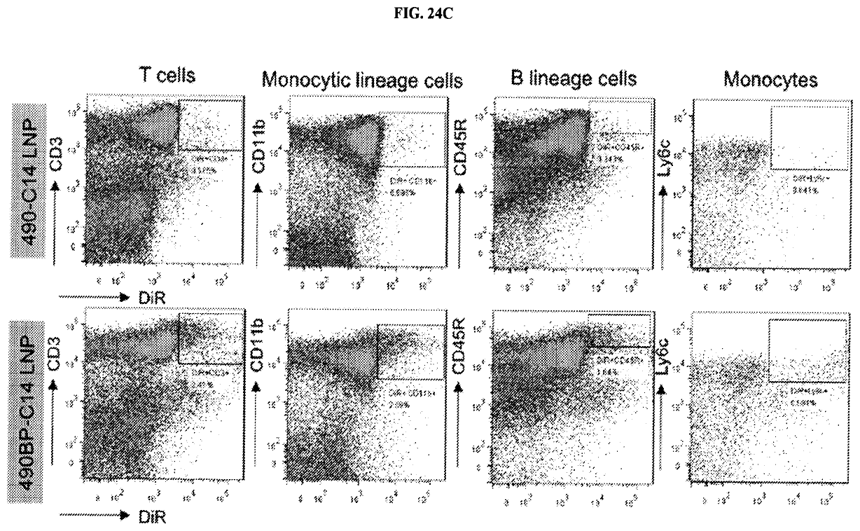

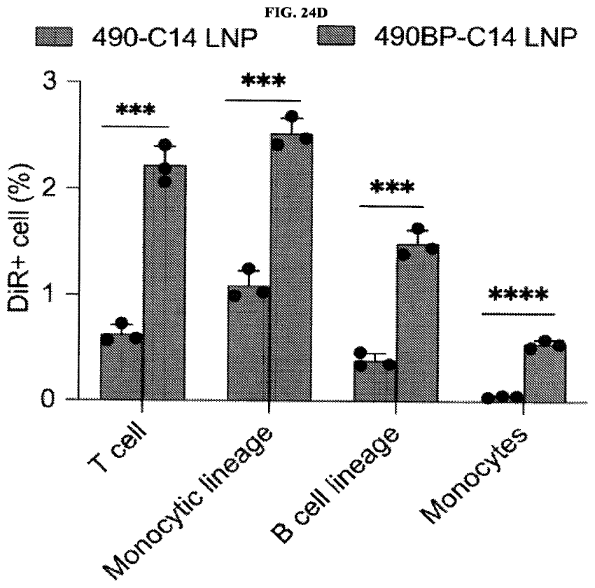

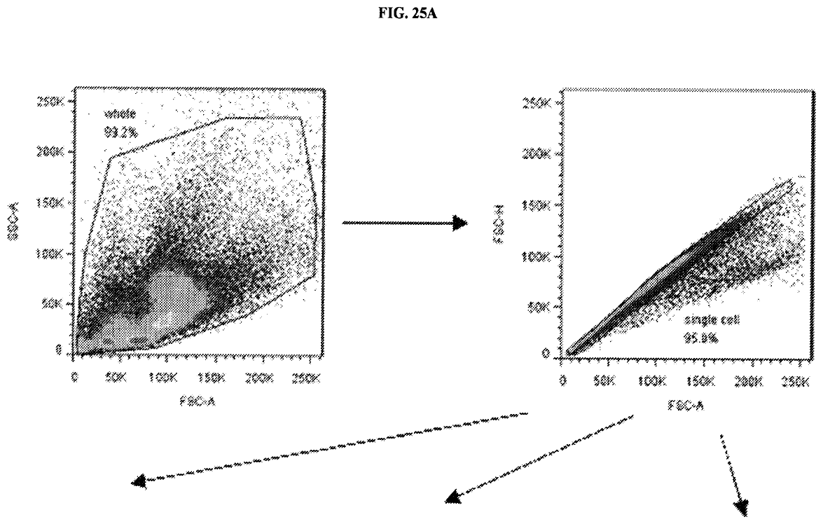

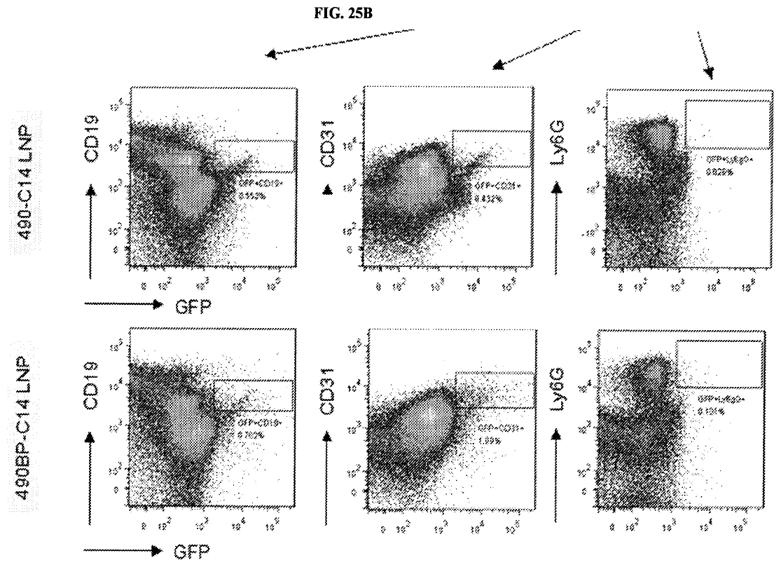

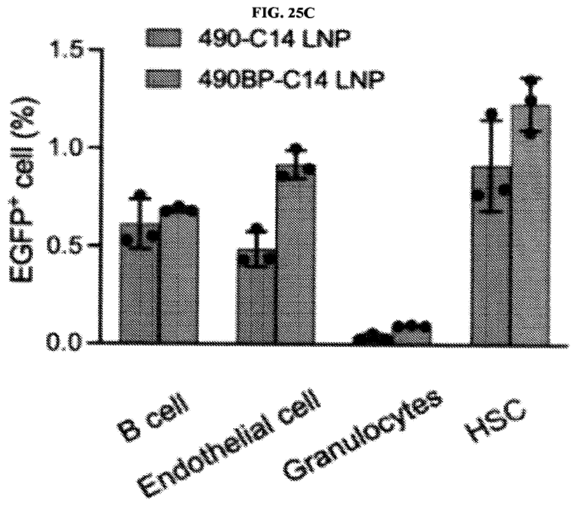

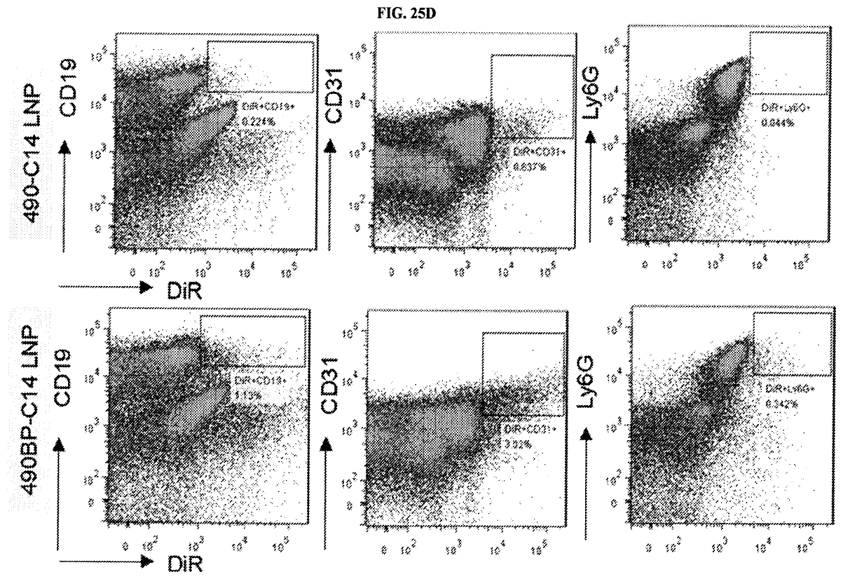

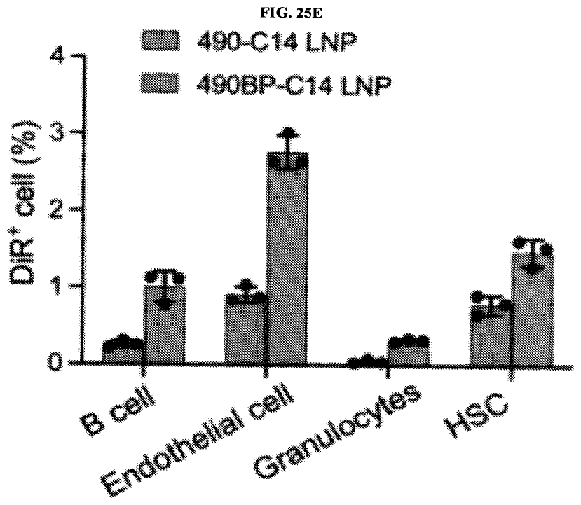

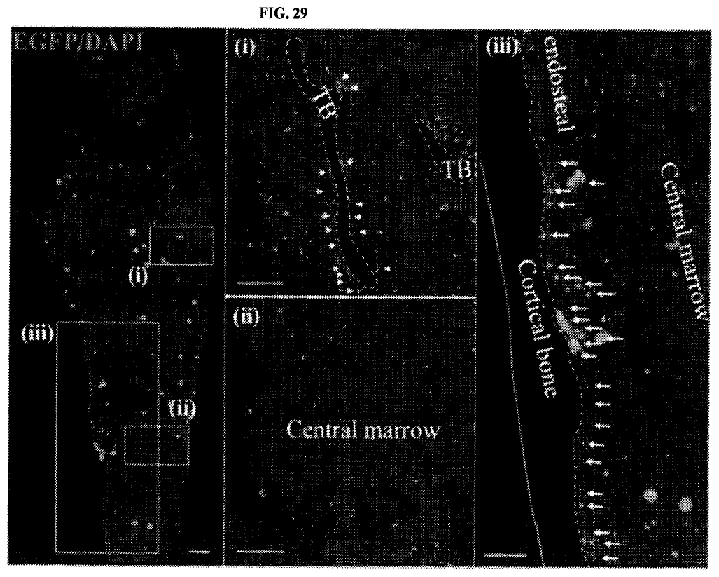

Having established that 490BP-C14 LNPs increased mRNA delivery to the bone microenvironment, the targeting of specific cell types in the bone marrow was evaluated, largely because bone marrow is the primary hematopoietic organ and residence site of diverse cell types. To demonstrate this, enhanced green fluorescent protein (EGFP)-encoding mRNA was administered to mice and flow cytometry was performed on cells isolated from bone marrow to identify transfection of different cell types. DiR labeled 490BP-C14 LNPs encapsulating EGFP mRNA were formulated and administered intravenously at 0.5 mg/kg, alongside PBS and 490-C14 LNP control groups (FIG. 29). After 12 h, mice were sacrificed, bone marrow was isolated, red blood cells were lysed, and the remaining cells were analyzed using flow cytometry. Flow cytometry analysis was conducted on diverse cell populations: monocytic lineage cells (CD11b⁺), B lineage cells (CD45R/B220⁺), T cells (CD3⁺), monocytes (Ly6c⁺), endothelial cells (CD31⁺), granulocytes (Ly6G) and B cells (CD19⁺) (FIGS. 24A-24D and FIGS. 25A-25E). Compared to the 490-C14 LNP treated group, EGFP transfection efficiency was enhanced in T cells, monocytic lineage cells and B lineage cells as well as a small increase in B cells, endothelial cells and granulocytes following delivery of BP-LNPs (FIGS. 24A-24B and FIGS. 25A-25E). Interestingly, 490BP-C14 LNPs exhibited over 4 times higher transfection efficiency to monocytes than 490-C14 LNPs in the absence of BPs (FIGS. 26A-26B), indicating their utility in applications in inflammatory associated diseases. In addition to quantification of mRNA functional delivery, LNP biodistribution to bone marrow cells was investigated. BP-LNPs increased accumulation in diverse cell types, such as T cells, monocytic lineage cells, B lineage cells and monocytes (FIGS. 24C-24D). The increased uptake was in agreement with the results from EGFP transfection efficiency (FIGS. 26A-26B), in which BP-LNP uptake by monocytes increased over 10 times compared with LNPs in the absence of BPs. Differing from the populations of EGFP⁺ cells, endothelial cells exhibited the highest BP-LNPs uptake ability, as a portion of BP-LNPs are not able to not pass through the blood-bone barrier after intravenous administration (FIGS. 25A-25E).

### Example 6: BMP-2 Secretion after Delivery of BMP-2 mRNA to Bone Microenvironment Using BP-LNPs

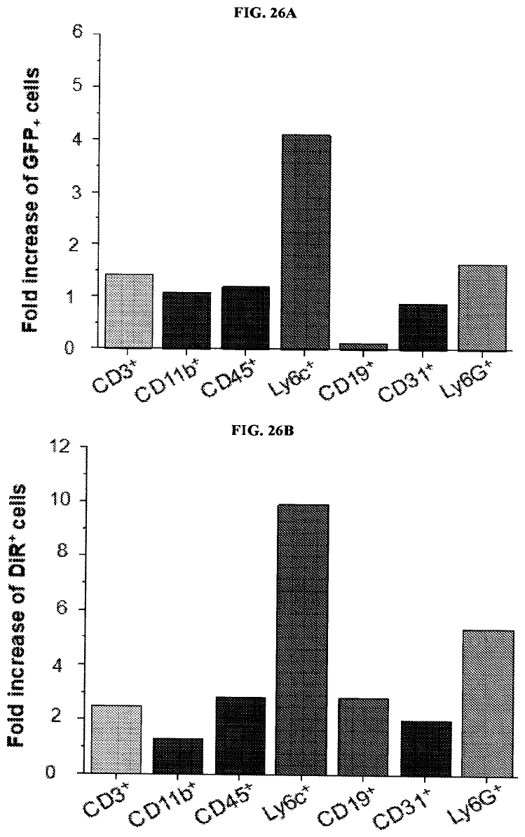

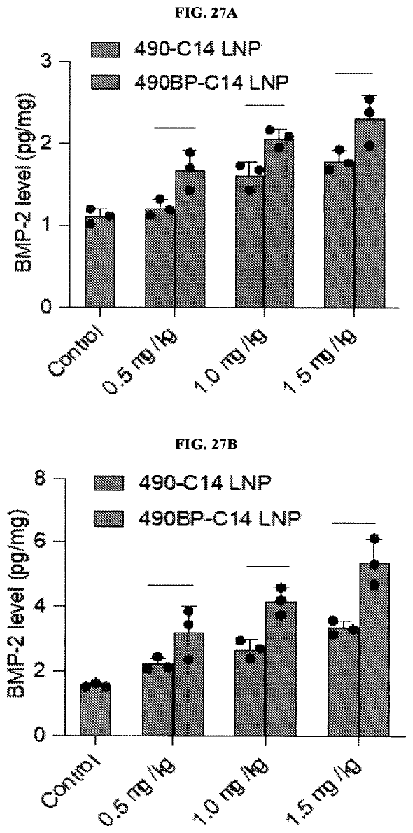

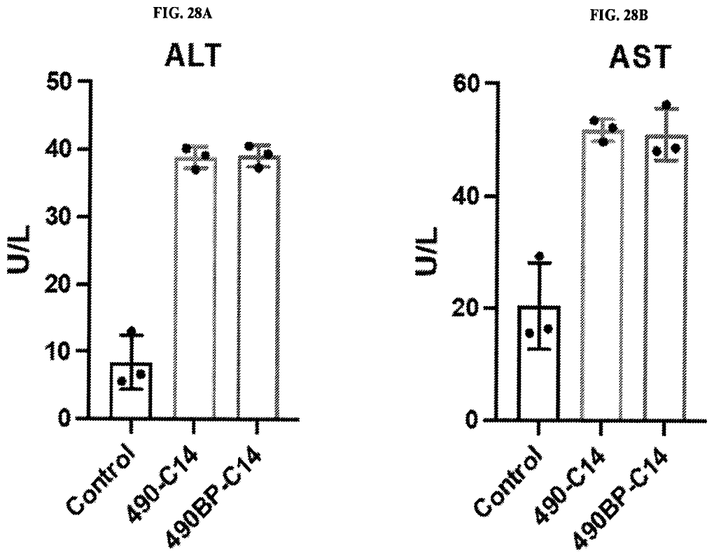

Having established BP-LNPs for enhanced systemic delivery of mRNA into the bone microenvironment, the delivery of mRNA encoding functional BMP-2 was then evaluated. BMP-2 is a multi-functional growth factor belonging to the transforming growth factor-beta (TGF-β) superfamily, which plays an important role in the process of bone induction that stimulates osteogenic differentiation. Therefore, increased expression of BMP-2 in the bone microenvironment leads to new applications for a variety of therapeutic interventions, such as bone defects, nonunion fractures, osteoporosis treatment, and root canal therapy. After BP-LNPs incorporating mRNA encoding BMP-2 were delivered by intravenous injection, BMP-2 levels on the bone surface and in the bone marrow were measured. It was noted that BMP-2 is normally secreted in the bone microenvironment, and thus BMP-2 secretion was observed both on the bone surface and in the bone marrow from the PBS treated control group. Although LNPs without BPs increased BMP-2 secretion in the bone microenvironment to some extent, BP-LNPs significantly increased BMP-2 expression (FIGS. 27A-27B). Furthermore, BMP-2 levels in the bone microenvironment increased with increasing BMP-2 mRNA dosage (FIGS. 27A-27B), reaching as high as 2.3 pg/mg and 5.3 pg/mg on the bone surface and in the bone marrow, respectively, at a mRNA dosage of 1.5 mg/kg, indicating great effectiveness for bone regeneration and fracture healing. Additionally, hematological analysis showed normal liver function (assessed by aspartate aminotransferase (AST) and alanine aminotransferase (ALT) activities) in mice treated with BP-LNPs, demonstrating that BP-LNPs did not cause notable toxicity in vivo (FIGS. 28A-28B).

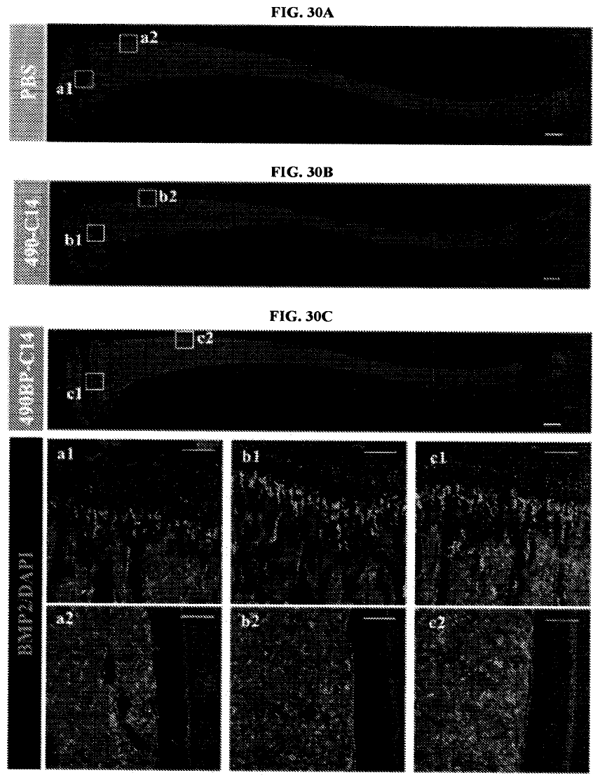

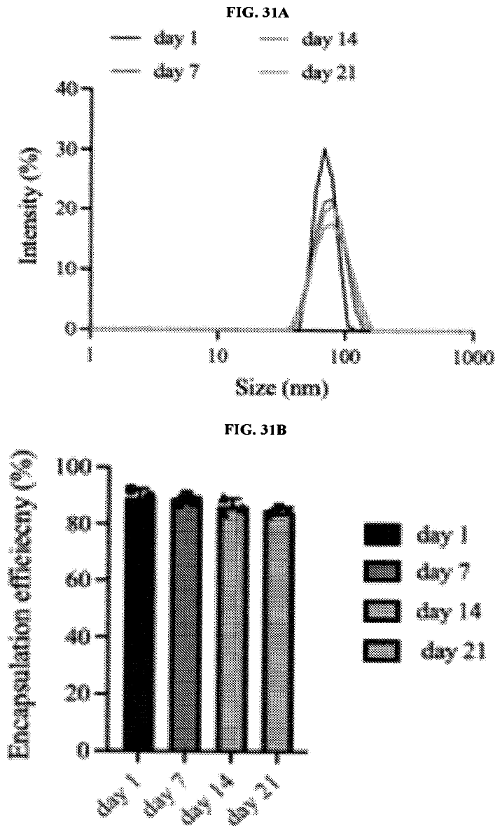

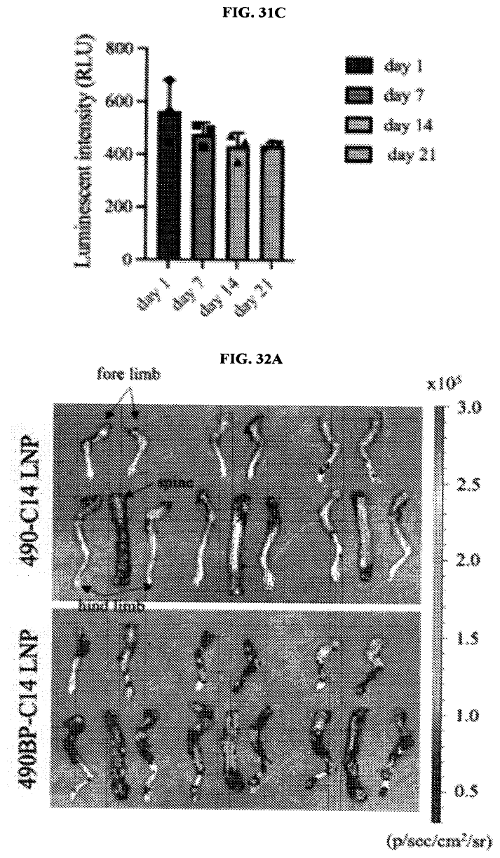

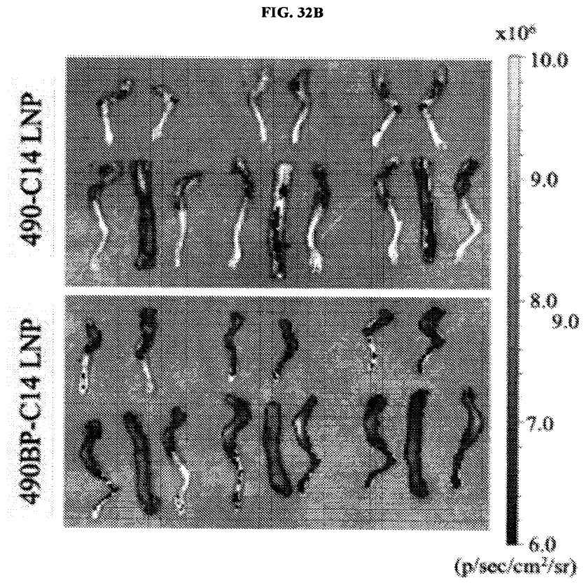

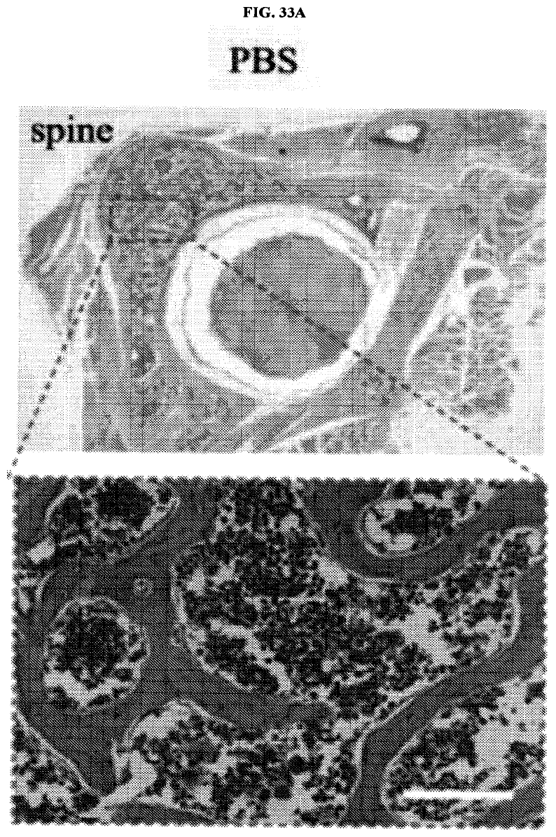

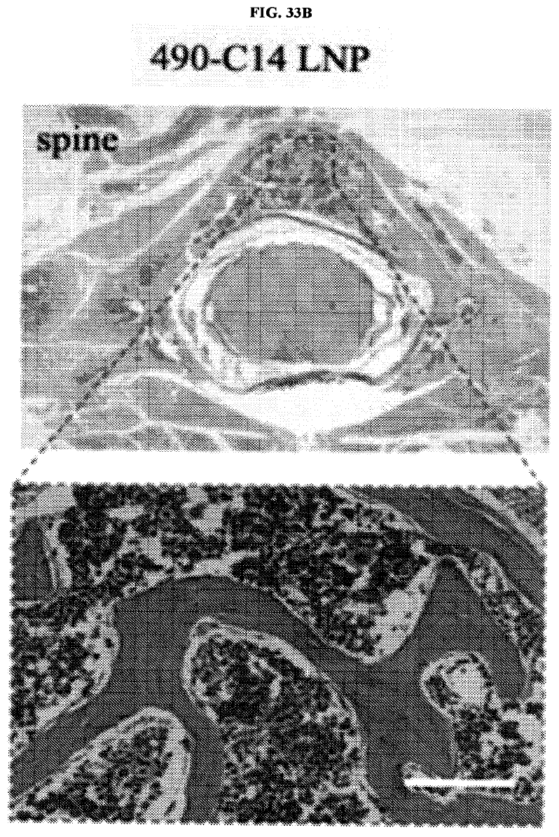

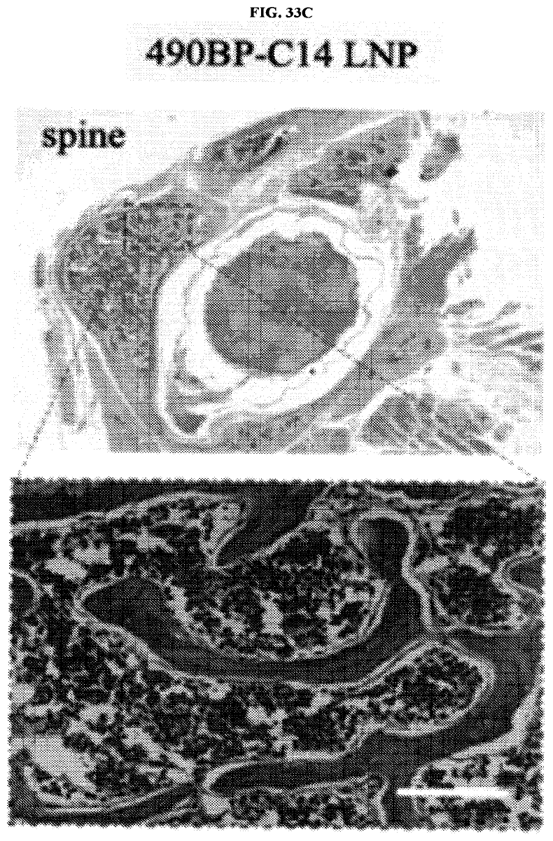

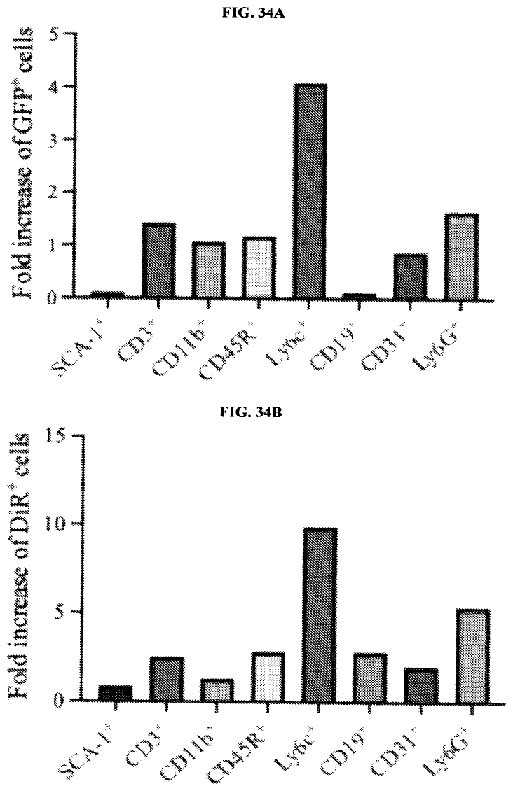

To further characterize BMP-2 expression, whole tibia bones from mice were then dissected for immunofluorescence (IF) analysis. Compared to low BMP-2 expression in mice treated by PBS (FIG. 30A), 490-C14 LNP delivering BMP-2 mRNA demonstrated relatively higher BMP-2 expression on both the trabecular and cortical bone surfaces (FIG. 30B). In addition, 490BP-C14 LNPs delivering BMP-2 mRNA demonstrated significantly higher BMP-2 expression in the metaphyseal and endosteal marrow (FIG. 30C). Representative BMP-2 expression regions, such as the growth plate, primary spongiosa, metaphyseal bone marrow (FIGS. 30A-30C; see a1, b1, and c1), and endosteal region (FIGS. 30A-30C; see a2, b2, and c2) of the medial anterior tibia were chosen to show the targeted delivery and expression of BMP-2. As shown in the magnified regions (FIGS. 30A-30C), the increase in BMP-2 expression in the metaphyseal and endosteal bone marrow for the 490BP-C14 LNP group illustrates the importance of BP lipid-like materials for mRNA delivery to bone and a potential disease treatment strategy based on BMP-2 expression.

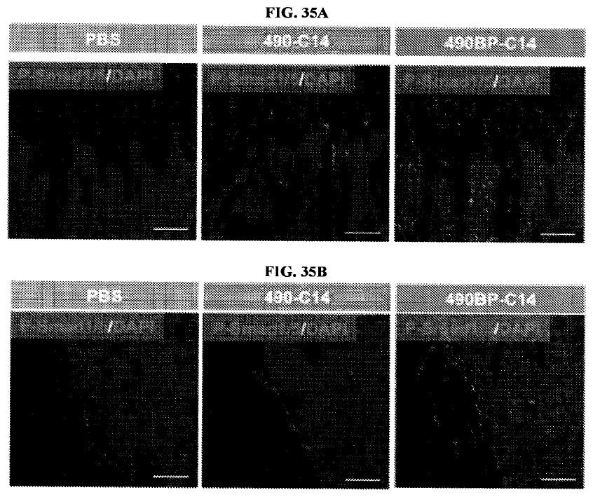

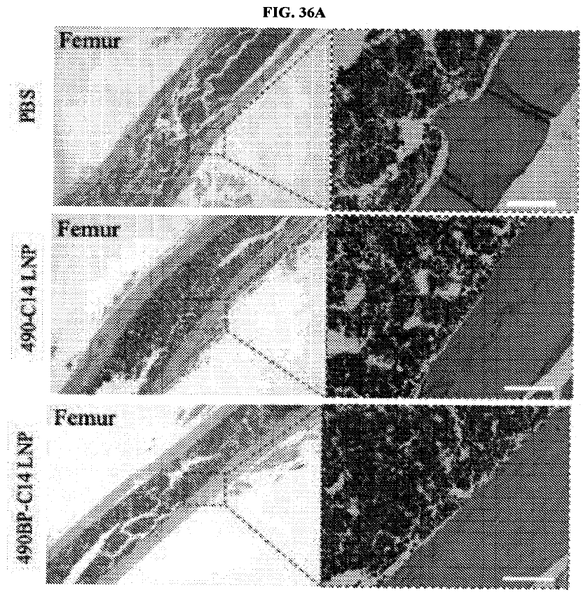

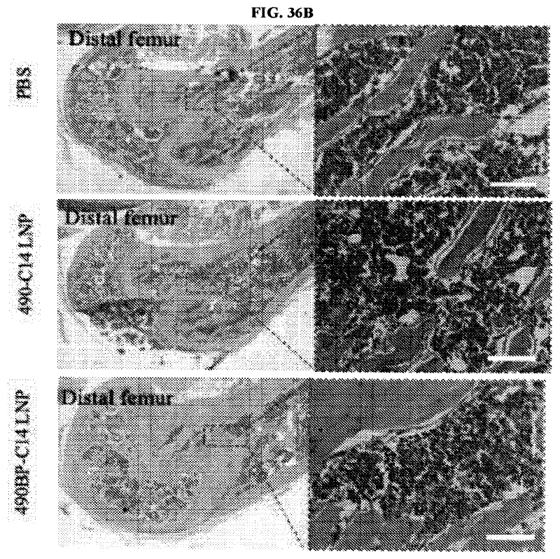

However, it is still difficult to characterize the cell populations which express BMP-2 because of (1) the diverse and complex cell populations in bone marrow and (2) the secretion of BMP-2 protein that may influence their internal distribution. To investigate whether proper signaling pathways are activated after BMP-2 mRNA delivery, IF staining of Smad1/5 was performed, which is phosphorylated during BMP-2 expression. A noticeable increase in phosphorylated Smad1/5 was detected in cells on the trabecular bone surface (FIG. 35A), on the cortical bone surface (FIG. 35B), and in bone marrow (FIGS. 35A-35B) after 490BP-C14 LNP delivery of BMP-2 mRNA, indicating efficient BMP-2 mRNA delivery to the bone microenvironment and subsequent signaling pathway activation.

In summary, a facile and versatile approach to engineer a library of BP lipid-like materials that were formulated into LNPs (BP-LNPs) for systemic delivery of mRNA to the bone microenvironment was developed. The bone-targeting BP ligand alendronate was readily conjugated onto amine cores to construct a series of novel BP lipid-like materials, which then formed stable bone-homing LNPs when combined with DOPE, cholesterol, and C14-PEG2000 through microfluidic mixing. After an initial assessment from in vitro screening, 490BP-C14 LNP was identified as the lead formulation in this library. Compared to LNPs that did not incorporate BP-lipids, 490BP-C14 LNPs showed increased higher hydroxyapatite binding in vitro and affinity to bone fragments ex vivo. Following systemic delivery, LNP homing and mRNA transfection in the bone microenvironment significantly increased with LNPs incorporating BP lipid-like materials. In addition, 490BP-C14 LNPs showed increased transfection efficiency in diverse cell types in the bone marrow, especially monocytes (4 times higher). BMP-2 secretion on the bone surface and in the bone marrow significantly increased after systemic BMP-2 mRNA delivery. The incorporation of BP lipid-like materials into LNPs demonstrated their effectiveness in targeted mRNA delivery to bone for applications in regenerative medicine, protein replacement, and gene editing therapies for bone and bone marrow. Additional studies focus on utilizing this generalizable approach to engineer targeted LNPs for enhanced site-specific targeting described here to develop expanded wealth of additional lipid-like materials for targeted mRNA therapeutics.

In conclusion, the development of LNP formulations for targeting the bone microenvironment holds significant utility for nucleic acid therapeutic applications, including bone regeneration, cancer, and hematopoietic stem cell therapies. However, therapeutic delivery to bone remained a significant challenge due to several biological barriers, such as low blood flow in bone, blood-bone marrow barriers, and low affinity between drugs and bone minerals, which led to unfavorable therapeutic dosages in the bone microenvironment. The present studies disclosed a series of BP lipid-like materials possessing high affinity to bone minerals, as a means to overcome biological barriers to deliver mRNA therapeutics efficiently to the bone microenvironment in vivo. Following in vitro screening of BP lipid-like materials formulated into LNPs, a lead BP-LNP formulation, 490BP-C14, was identified with enhanced mRNA expression and localization in the bone microenvironment of mice in vivo compared to 490-C14 LNPs in the absence of BPs. Moreover, BP-LNPs enhanced mRNA delivery and secretion of therapeutic bone morphogenetic protein-2 from the bone microenvironment upon intravenous administration. These results demonstrated the effectiveness of BP-LNPs for delivery to the bone microenvironment, which are utilized for a range of mRNA therapeutic applications, including regenerative medicine, protein replacement, and gene editing therapies.

Moreover, the materials of the present disclosure are useful in delivering nucleic acids into bones. LNPs commonly go to liver via IV injection and extra-hepatic delivery remains a daunting challenge. The bone marrow microenvironment is rich in genetic targets, such as hematopoietic stem cells (HSCs), and cancer cells that metastasize to bone. Furthermore, bone regeneration is a major challenge that mRNA therapeutics of the present disclosure address. The herein described bone-targeted ionizable lipids are also able to deliver nucleic acids to HSCs for gene editing therapies, to cancer cells for immunotherapy or gene therapies, and are also able to produce proteins to promote bone regeneration.

### ENUMERATED EMBODIMENTS

The following exemplary embodiments are provided, the numbering of which is not to be construed as designating levels of importance:

Embodiment 1 provides a lipid nanoparticle (LNP). In certain embodiments, the LNP comprises (a) at least one compound having the structure of Formula (I), or a racemate, enantiomer, diastereomer, pharmaceutically acceptable salt, solvate, or a derivative thereof:

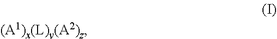

wherein:
each occurrence of A¹ is independently

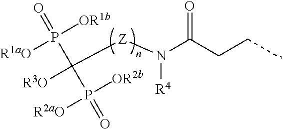

each occurrence of A² is independently

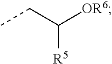

each occurrence of L is an amine linker independently selected from the group consisting of aminoalkyl linker, substituted aminoalkyl linker, diaminoalkyl linker, substituted diaminoalkyl linker, triaminoalkyl linker, substituted triaminoalkyl linker, tetraaminoalkyl linker, substituted tetraaminoalkyl linker, pentaaminoalkyl linker, substituted pentaaminoalkyl linker, polyaminoalkyl linker, substituted polyaminoalkyl linker, aminocycloalkyl linker, substituted aminocycloalkyl linker, diaminocycloalkyl linker, substituted diaminocycloalkyl linker, triaminocycloalkyl linker, substituted triaminocycloalkyl linker, tetraaminocycloalkyl linker, substituted tetraaminocycloalkyl linker, pentaaminocycloalkyl linker, substituted pentaaminocycloalkyl linker, poly aminocycloalkyl linker, substituted polyaminocycloalkyl linker, and any combination thereof;
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si;
each occurrence of z′ and z″ is independently an integer represented by 0, 1, or 2;
wherein x, y, and z are independently an integer from 0 to 20;
each occurrence of n is independently an integer from 0 to 10; and
the compound is present in a concentration range of about 1 mol % to about 99 mol %. In certain embodiments, the LNP comprises (b) at least one neutral phospholipid, wherein the neutral phospholipid is present in a concentration range of about 5 mol % to about 45 mol %. In certain embodiments, the LNP comprises (c) at least one cholesterol lipid, wherein the total cholesterol lipid is in a concentration range of about 5 mol % to about 55 mol %. In certain embodiments, the LNP comprises (d) at least one polymer conjugated lipid (e.g., polyethylene glycol (PEG)-conjugated lipid), wherein the total polymer conjugated lipid is present in a concentration range of about 0.5 mol % to about 12.5 mol %.

Embodiment 2 provides the LNP of Embodiment 1, wherein L is selected from the group consisting of

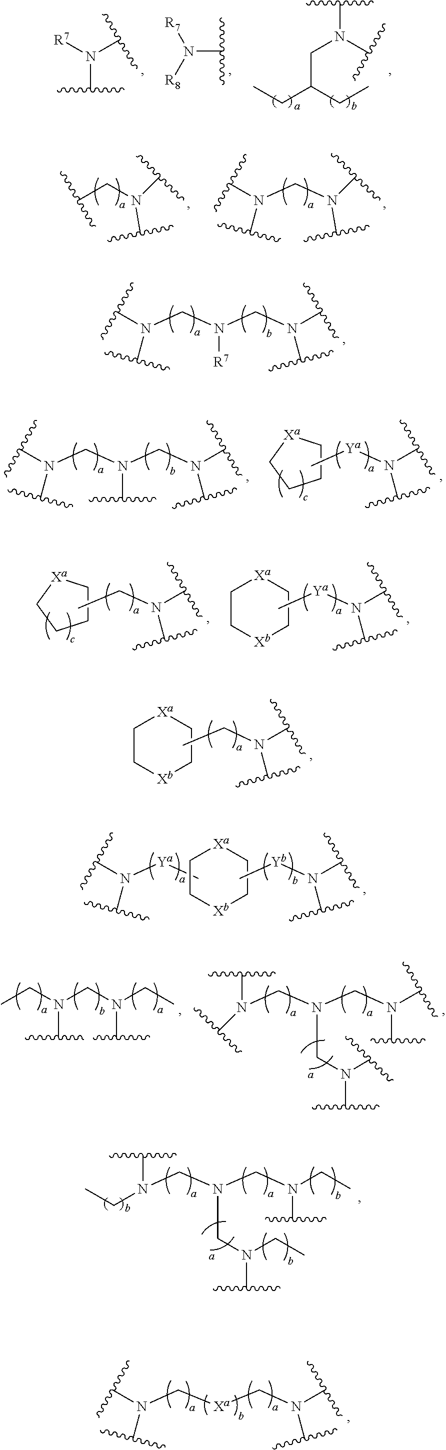

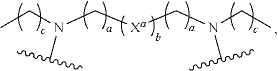

and any combination thereof,
wherein:
each occurrence of R⁷ and R⁸ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of X^{a} and X^{b} is independently selected from the group consisting of O, S, N(R⁹)_{z′}, P(R⁹)_{z′}, and any combination thereof;
each occurrence of Y^{a} and Y^{b} is independently selected from the group consisting of C₁-C₁₂ alkylenyl, substituted C₁-C₁₂ alkylenyl, C₃-C₈ cycloalkylenyl, substituted C₃-C₈ cycloalkylenyl, —Y′(R⁹)_{z′}(R¹⁰)_{z″}—(C₃-C₈ cycloalkylenyl), substituted —Y′(R⁹)_{z′}(R¹⁰)_{z″}—C₃-C₈ cycloalkylenyl, C₂-C₈ heterocycloalkylenyl, substituted C₂-C₈ heterocycloalkylenyl, —Y′(R⁹)_{z′}(R¹⁰)_{z″}—C₂-C₈ heterocycloalkylenyl, substituted-Y′(R⁹)_{z′}(R¹⁰)_{z″}—C₂-C₈ heterocycloalkylenyl, C₂-C₈ alkenylenyl, substituted C₂-C₈ alkenylenyl, C₅-C₁₀ cycloalkenylenyl, substituted C₅-C₁₀ cycloalkenylenyl, —Y′(R⁹)_{z′}(R¹⁰)_{z″}—C₅-C₁₀ cycloalkenylenyl, substituted —Y′(R⁹)_{z′}(R¹⁰)_{z″}—C₅-C₁₀ cycloalkenylenyl, C₂-C₈ alkynylenyl, substituted C₂-C₈ alkynylenyl, C₈-C₁₂ cycloalkynylenyl, substituted C₈-C₁₂ cycloalkynylenyl, —Y′(R⁹)_{z′}(R¹⁰)_{z″}—C₈-C₁₂ cycloalkynylenyl, substituted —Y′(R⁹)_{z′}(R¹⁰)_{z″}—C₅-C₁₂ cycloalkynylenyl, C₆-C₁₀ arylenyl, substituted C₆-C₁₀ arylenyl, —Y′(R⁹)_{z′}(R¹⁰)_{z″}—C₆-C₁₀ arylenyl, substituted —Y′(R⁹)_{z′}(R¹⁰)_{z″}—C₆-C₁₀ arylenyl, C₂-C₁₀ heteroarylenyl, substituted C₂-C₁₀ heteroarylenyl, —Y′(R⁹)_{z′}(R¹⁰)_{z″}—C₂-C₁₀ heteroarylenyl, and substituted —Y′(R⁹)_{z′}(R¹⁰)_{z″}—C₂-C₁₀ heteroarylenyl;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2;
wherein each occurrence of a, b, and c is independently an integer from 0 to 10;
each occurrence of

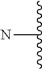 [SMILES: *N]

indicates a bond between a N atom of L and A¹ or A².

Embodiment 3 provides the LNP of Embodiment 1 or 2, wherein L is selected from the group consisting of

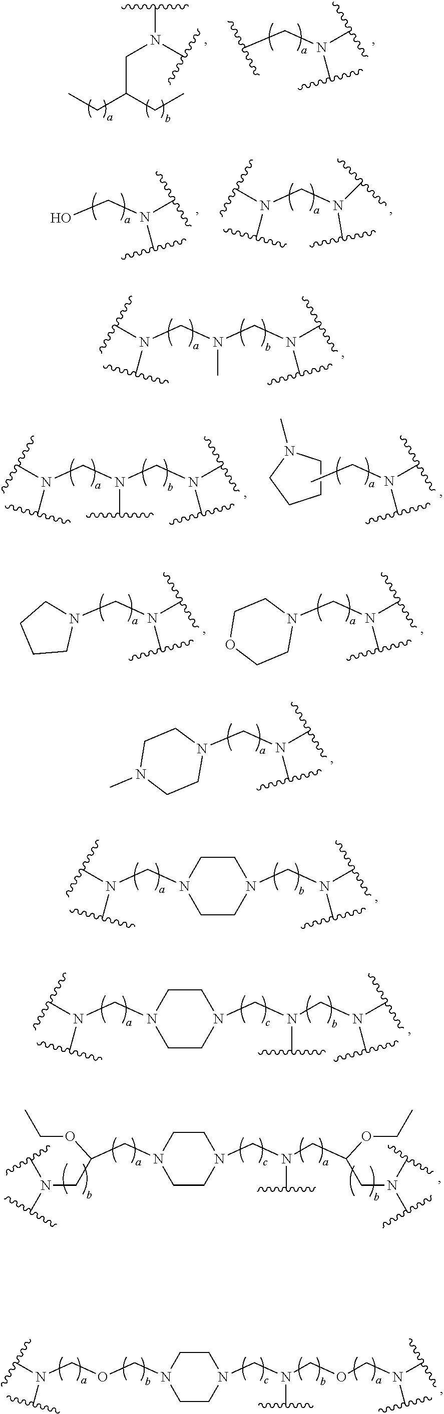

any combination thereof,
wherein:
each occurrence of a b, and c is independently an integer from 0 to 10; and
each occurrence of

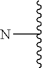 [SMILES: *N]

indicates a bond between a N atom of L and A¹ or A².

Embodiment 4 provides the LNP of any one of Embodiments 1-3, wherein the compound having the structure of Formula (I) is a compound having the structure selected from the group consisting of:

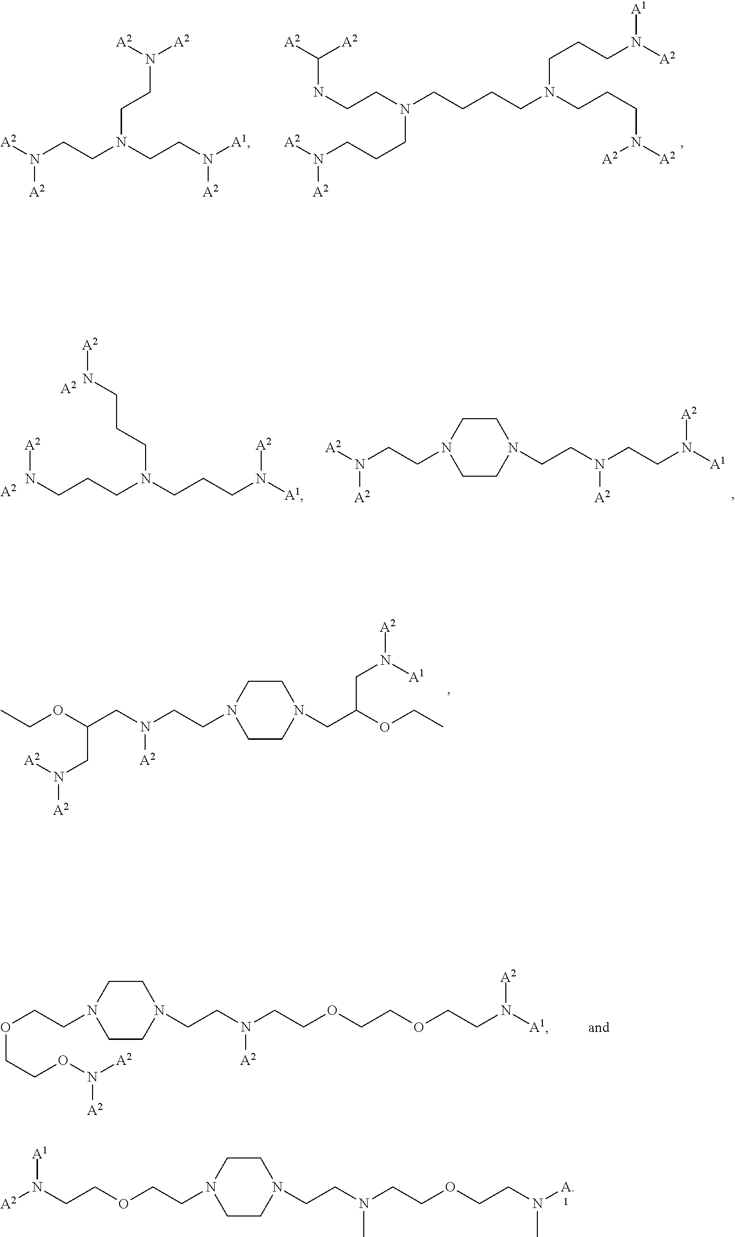

Embodiment 5 provides the LNP of Embodiment 4, wherein A¹ is

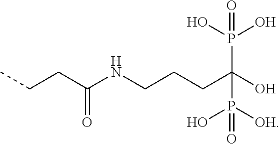

Embodiment 6 provides the LNP of Embodiment 4 or 5, wherein each occurrence of A² is independently selected from the group consisting of

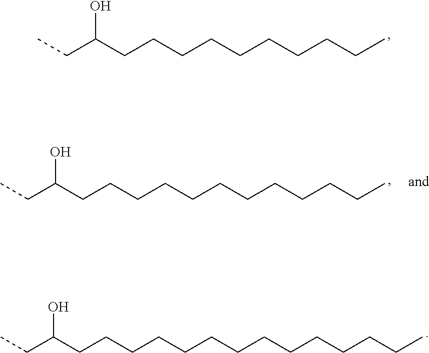

Embodiment 7 provides the LNP of any one of Embodiments 1-7, wherein the compound having the structure of Formula (I) is a compound having the structure selected from the group consisting of:

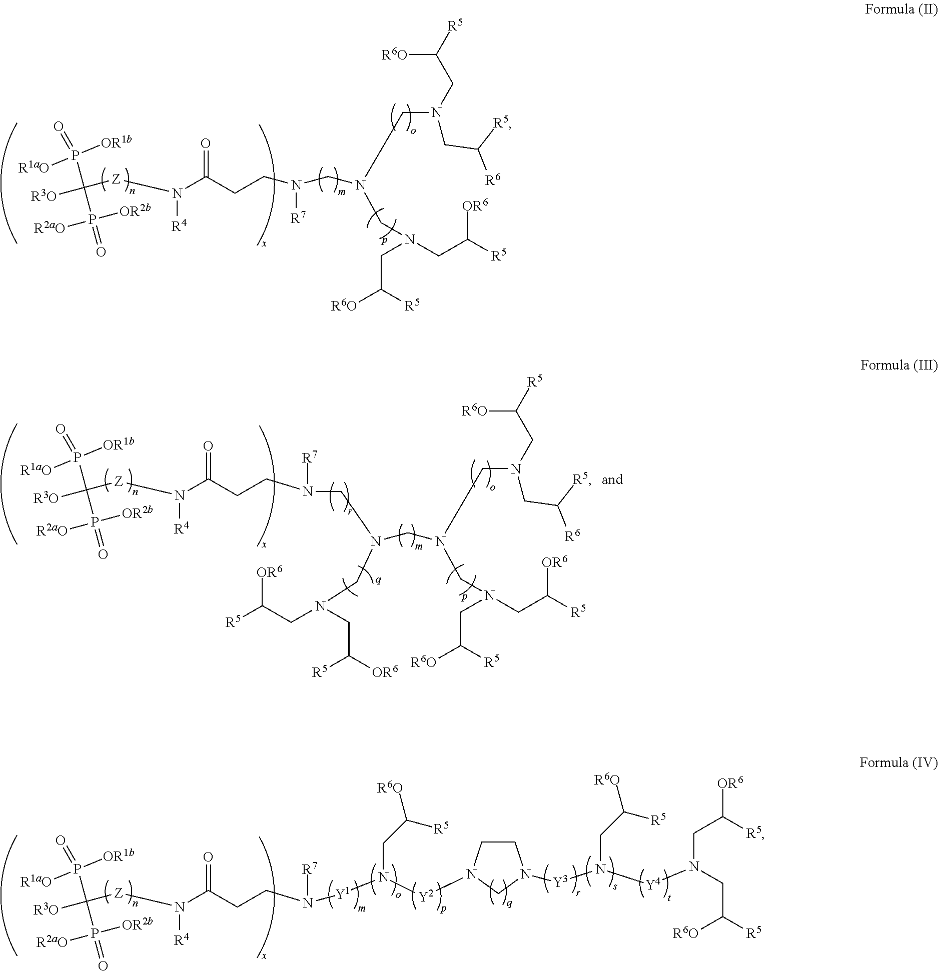

wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl. and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′} (R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof;
each occurrence of Y¹, Y², Y³, and Y⁴ is independently selected from the group consisting of O, S, C(R⁹)_{z′}(R¹⁰)_{z″}, N(R⁹)_{z′}, P(R⁹)_{z′}, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-CLO aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5.

Embodiment 8 provides the LNP of any one of Embodiments 1-7, wherein the compound having the structure of Formula (I) is a compound having the structure selected from the group consisting of

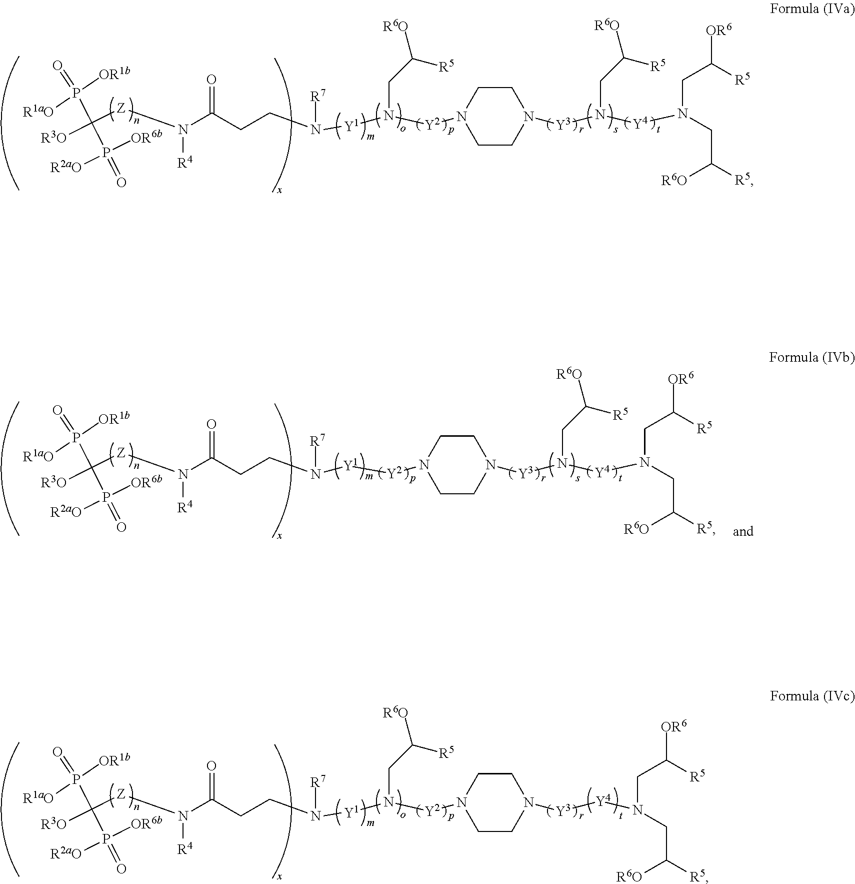

wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₉-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z′}-ester, —Y(R⁹)_{z′}(R¹⁰)_{z″}, —NO₂, —CN, ═O, ═S, sulfoxy, sulfonate, sulfate, sulfite, and sulfide, and any combination thereof;
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}—NO₂, —CN, sulfoxy, sulfonate, sulfate, sulfite, and sulfide, and any combination thereof;
each occurrence of Y¹, Y², Y³, and Y⁴ is independently selected from the group consisting of O, S, C(R⁹)_{z′}(R¹⁰)_{z″}, N(R⁹)_{z′}, P(R⁹)_{z′}, and any combination thereof; each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-CLO aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5.

Embodiment 9 provides the LNP of any one of Embodiments, wherein the compound having the structure of Formula (I) is a compound having the structure selected from the group consisting of

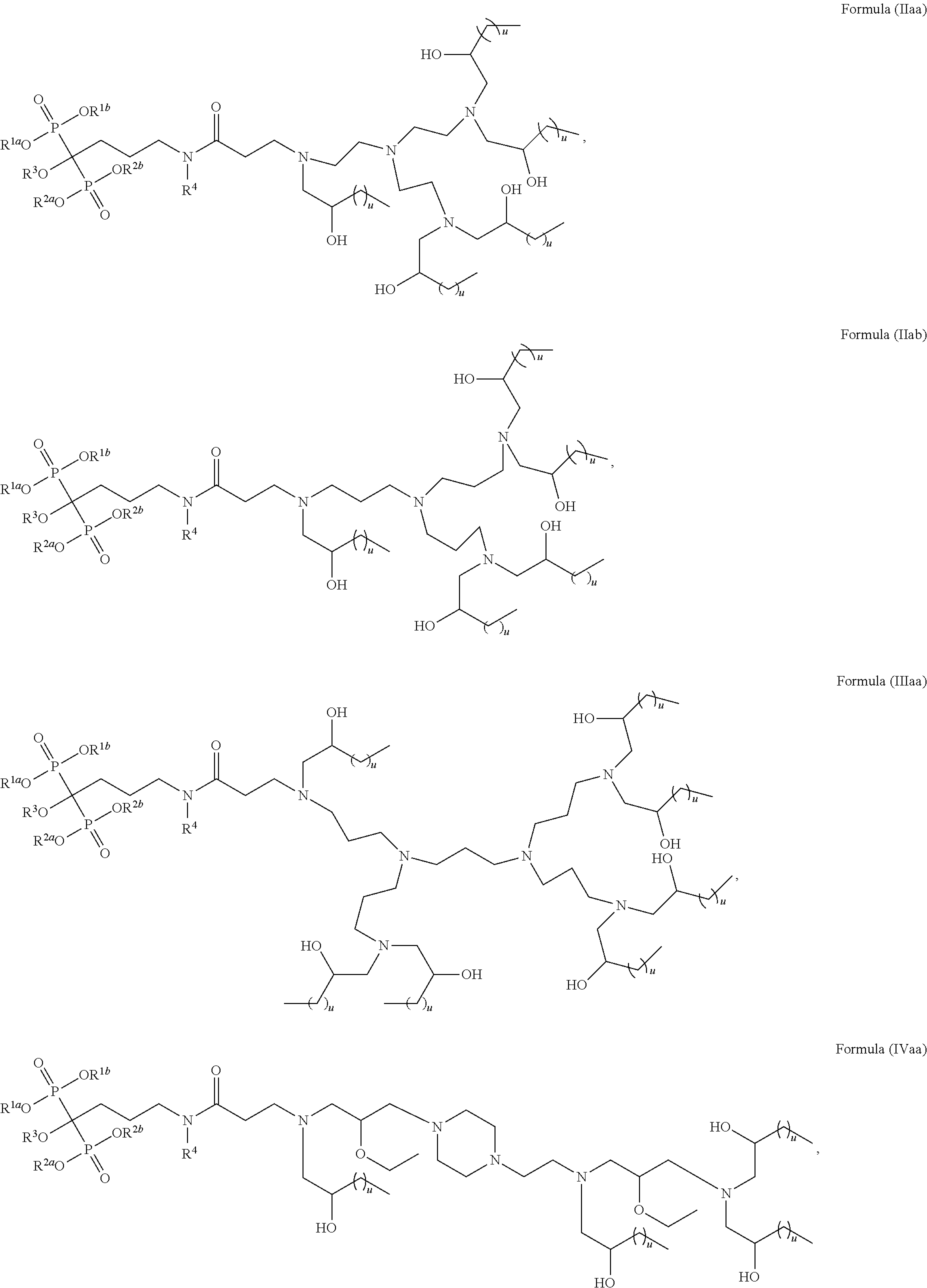

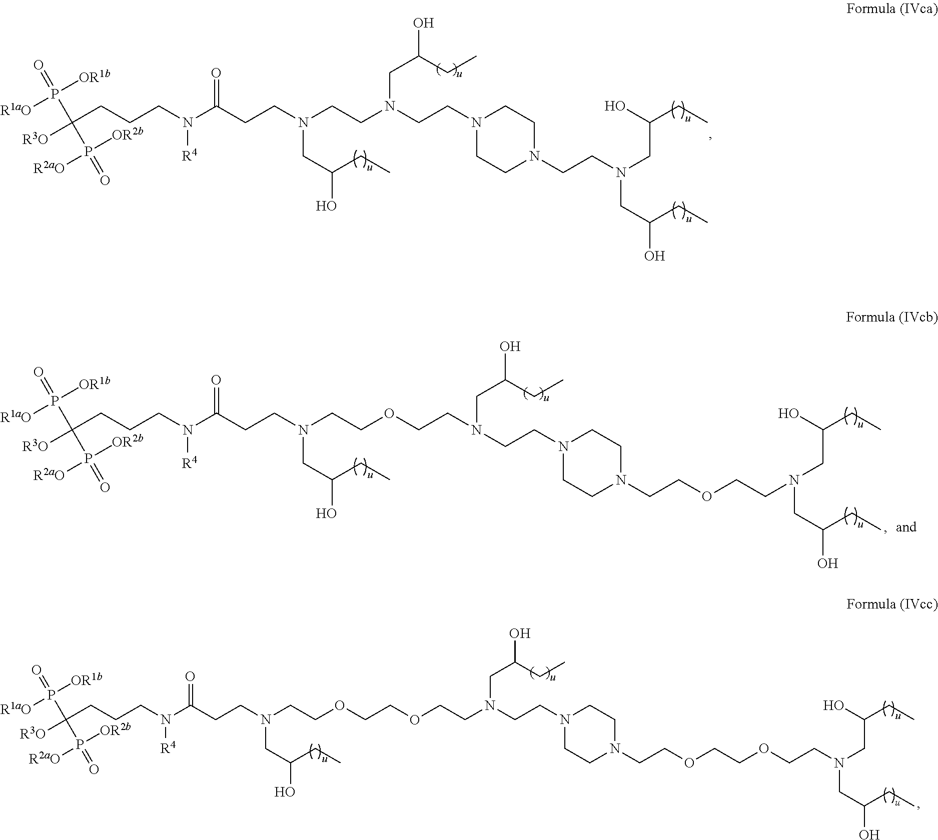

wherein:
each occurrence of R^{1a}, R^{1b}, R^{2a}, R^{2b}, R³, and R⁴ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of R⁹ and R¹⁰ is independently selected from the group consisting of halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein u is an integer from 0 to 20.

Embodiment 10 provides the LNP of any one of Embodiments 1-9, wherein the compound having the structure of Formula (I) is selected from the group consisting of

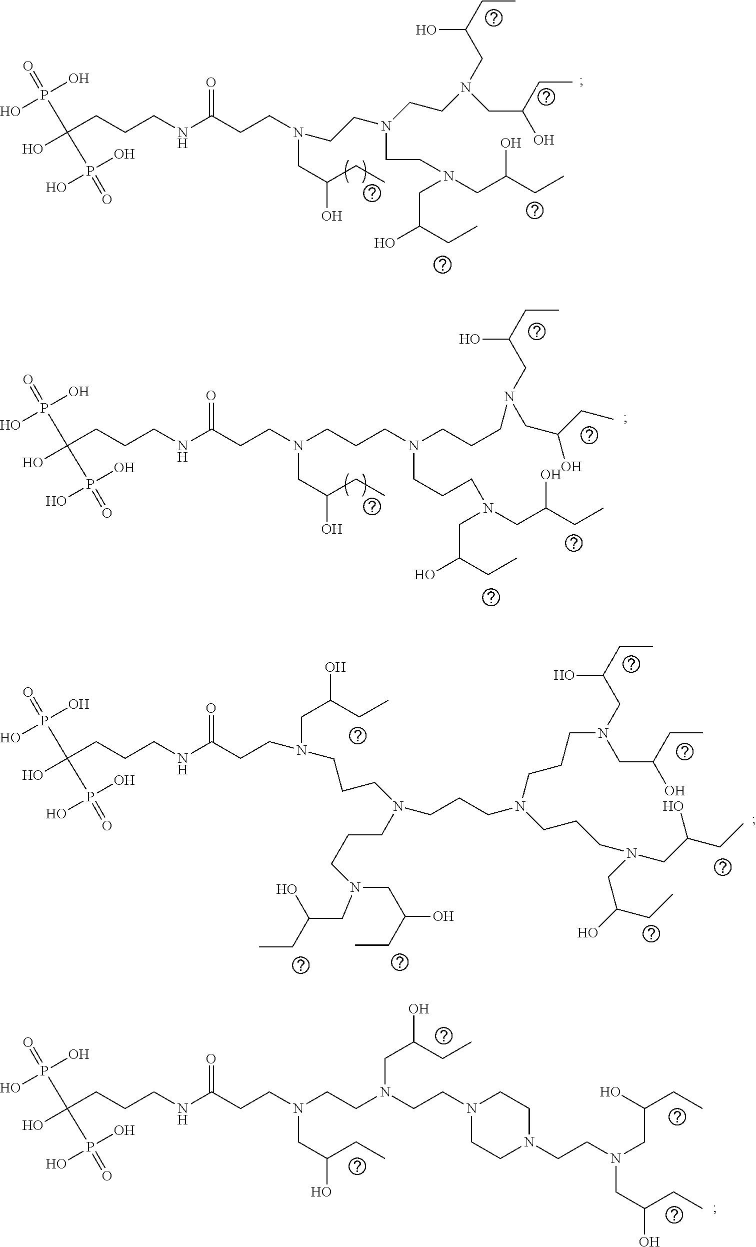

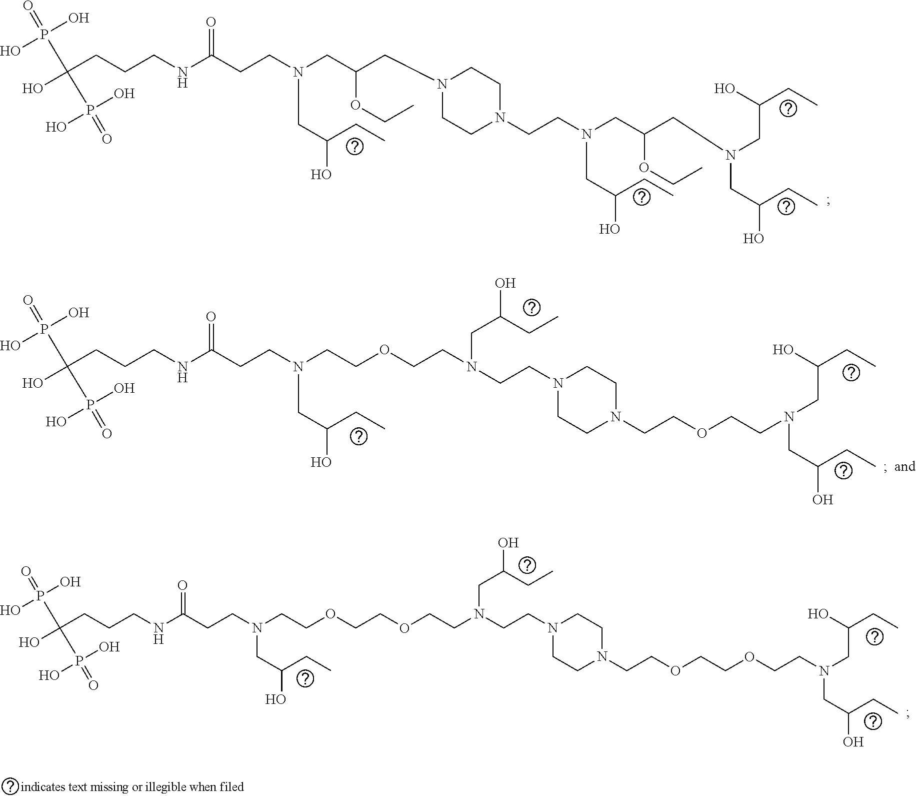

wherein u is an integer from 5 to 15.

Embodiment 11 provides the LNP of any one of Embodiments 1-10, wherein the compound having the structure of Formula (I) is:

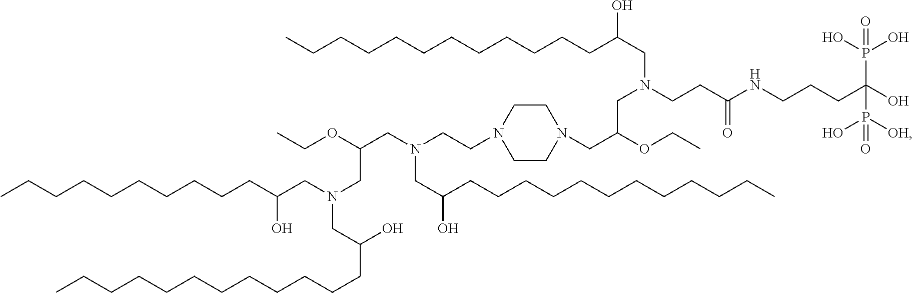 [SMILES: CCCCCCCCCCCCC(O)CN(CCC(=O)NCCCC(O)(P(=O)(O)O)P(=O)(O)O)CC(CN1CCN(CCN(CC(O)CCCCCCCCCCCC)CC(CN(CC(O)CCCCCCCCCC)CC(O)CCCCCCCCCCCC)OCC)CC1)OCC]

(4-(3-((3-(4-(2-((3-(bis(2-hydroxytetradecyl)amino)-2-ethoxypropyl)(2-hydroxytetradecyl)amino)ethyl)piperazin-1-yl)-2-ethoxypropyl)(2-hydroxytetradecyl)amino)propanamido)-1-hydroxybutane-1,1-diyl)bis(phosphonic acid).

Embodiment 12 provides the LNP of any one of Embodiments 1-11, wherein the at least one neutral phospholipid comprises at least one selected from the group consisting of dioleoyl-phosphatidylethanolamine (DOPE), dioleoylphosphatidylcholine (DOPC), distearoylphosphatidylcholine (DSPC), distearoyl-phosphatidylethanolamine (DSPE), 16-O-dimethyl PE, 18-1-trans PE, 1-stearioyl-2-oleoyl-phosphatidyethanol amine (SOPE), stearoyloleoylphosphatidylcholine (SOPC), 2-dioleoyl-3-trimethylammonium-propane (DOTAP), and any combination thereof.

Embodiment 13 provides the LNP of any one of Embodiments 1-12, wherein the at least one cholesterol lipid comprises at least one selected from the group consisting of a cholesterol, cholesteryl derivate, and any combination thereof.

Embodiment 14 provides the LNP of any one of Embodiments 1-13, wherein the at least one polymer conjugated lipid is a PEG conjugated lipid.

Embodiment 15 provides the LNP of Embodiment 14, wherein the PEG-conjugated lipid comprises at least one selected from the group consisting of 1,2-dimyristoyl-sn-glycero-3-phosphoethanolamine-N-[methoxy(polyethylene glycol)-2000](C14-PEG2000), C12-PEG2000, C12-PEG490, 1,2-dimyristoyl-rac-glycero-3-methoxypolyethylene glycol-2000 (DMG-PEG2000), 1,2-distearoyl-sn-glycero-3-phosphoethanolamine-N-[amino(polyethylene glycol)-2000](DSPE-PEG 2000 amine), and any combination thereof.

Embodiment 16 provides the LNP of any one of Embodiments 1-15, wherein the molar ratio of (a):(b):(c):(d) is about 1-80%:5-45%:5-55%:0.5-12.5%.

Embodiment 17 provides the LNP of any one of Embodiments 1-16, wherein the molar ratio of (a):(b):(c):(d) is about 35-45%:5-20%:40-55%:1-2.5%.

Embodiment 18 provides the LNP of any one of Embodiments 1-17, wherein the molar ratio of (a):(b):(c):(d) is about 30-35%:16%:46.5%:2.5%.

Embodiment 19 provides the LNP of any one of Embodiments 1-18, wherein the LNP has a diameter of between about 10 nm to about 1000 nm.

Embodiment 20 provides the LNP of any one of Embodiments 1-19, wherein the LNP has a diameter of between about 50 nm to about 500 nm.

Embodiment 21 provides the LNP of any one of Embodiments 1-20, wherein the LNP selectively binds to at least one target cell of interest.

Embodiment 22 provides the LNP of Embodiment 21, wherein the at least one target cell of interest is selected from the group consisting of a stem cell, bone cell, bone marrow cell, and any combination thereof.

Embodiment 23 provides the LNP of Embodiment 21 or 22, wherein the at least one target cell of interest is selected from the group consisting of a stem cell, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, and any combination thereof.

Embodiment 24 provides the LNP of any one of Embodiments 1-23, wherein the LNP further comprises or encapsulates at least one agent.

Embodiment 25 provides the LNP of Embodiment 24, wherein the weight ratio of (a): the at least one agent is between about 1:1 to about 10:1.

Embodiment 26 provides the LNP of Embodiment 24, wherein the agent is selected from the group consisting of a nucleic acid molecule, a small molecule, a protein, an antibody, and any combination thereof.

Embodiment 27 provides the LNP of Embodiment 26, wherein the nucleic acid molecule is a DNA molecule or an RNA molecule.

Embodiment 28 provides the LNP of Embodiment 26, wherein the nucleic acid molecule is selected from the group consisting of cDNA, mRNA, miRNA, siRNA, sgRNA, modified RNA, CRISPR-Cas9, antagomir, antisense molecule, targeted nucleic acid, and any combination thereof.

Embodiment 29 provides the LNP of Embodiment 24, wherein the LNP has a diameter of between about 10 nm to about 1000 nm.

Embodiment 30 provides the LNP of Embodiment 29, wherein the LNP has a diameter of between about 50 nm to about 500 nm.

Embodiment 31 provides the LNP of any one of Embodiments 26-30, wherein the LNP is suitable for delivering a nucleic acid to a bone cell, bone marrow cell, or a combination thereof.

Embodiment 32 provides a composition comprising at least one LNP of any one of Embodiments 1-31, optionally further comprising at least one pharmaceutically acceptable excipient.

Embodiment 33 provides the composition of Embodiment 32, wherein the composition is suitable for delivering a nucleic acid to a bone cell, bone marrow cell, or a combination thereof.

Embodiment 34 provides a method of delivering an agent to a subject in need thereof, the method comprising administering a therapeutically effectively amount of at least one LNP of any one of Embodiments 1-31 and/or the composition of Embodiment 32 or 33 the same to the subject.

Embodiment 35 provides the method of Embodiment 34, wherein the agent is selected from the group consisting of a nucleic acid molecule, a small molecule, a protein, an antibody, a therapeutic agent, and any combination thereof.

Embodiment 36 provides the method of Embodiment 35, wherein the nucleic acid molecule is a DNA molecule or an RNA molecule.

Embodiment 37 provides the method of Embodiment 35, wherein the nucleic acid molecule is selected from the group consisting of cDNA, mRNA, miRNA, siRNA, sgRNA, modified RNA, CRISPR-Cas9, antagomir, antisense molecule, targeted nucleic acid, and any combination thereof.

Embodiment 38 provides the method of Embodiment 35, wherein the LNP selectively binds to at least one target cell of interest.

Embodiment 39 provides the method of Embodiment 38, wherein the at least one target cell of interest is selected from the group consisting of a stem cell, bone cell, bone marrow cell, and any combination thereof.

Embodiment 40 provides the method of Embodiment 38 or 39, wherein the at least one target cell of interest is selected from the group consisting of a stem cell, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, and any combination thereof.

Embodiment 41 provides the method of Embodiment 34, wherein the agent is encapsulated within the LNP.

Embodiment 42 provides the method of Embodiment 34, wherein the LNP or the composition comprising the same is administered intravenously, intramuscularly, intradermally, subcutaneously, intranasally, by inhalation, or any combination thereof.

Embodiment 43 provides a method of delivering an agent to a bone in a subject, bone marrow, or a combination thereof, the method comprising administering a therapeutically effectively amount of at least one LNP of any one of Embodiments 1-31 and/or the composition of Embodiment 32 or 33 to the subject.

Embodiment 44 provides the method of Embodiment 43, wherein the agent is selected from the group consisting of a nucleic acid molecule, a small molecule, a protein, an antibody, a therapeutic agent, and any combination thereof.

Embodiment 45 provides the method of Embodiment 44, wherein the nucleic acid molecule is a DNA molecule or an RNA molecule.

Embodiment 46 provides the method of Embodiment 44, wherein the nucleic acid molecule is selected from the group consisting of cDNA, mRNA, miRNA, siRNA, sgRNA, modified RNA, CRISPR-Cas9, antagomir, antisense molecule, targeted nucleic acid, and any combination thereof.

Embodiment 47 provides the method of Embodiment 43, wherein the LNP selectively binds to at least one bone cell, bone marrow cell, or a combination thereof.

Embodiment 48 provides the method of Embodiment 43, wherein the agent is encapsulated within the LNP.

Embodiment 49 provides the method of Embodiment 43, wherein the LNP or the composition comprising the same is administered intravenously, intramuscularly, intradermally, subcutaneously, intranasally, by inhalation, or any combination thereof.

Embodiment 50 provides a method of treating, ameliorating, and/or preventing at least one disease, disorder, or condition in a subject in need thereof, the method comprising administering a therapeutically effectively amount of at least one LNP of any one of Embodiments 1-31 or the composition of Embodiment 32 or 33 the same to the subject.

Embodiment 51 provides the method of Embodiment 50, wherein the disease, disorder, or condition is selected from the group consisting of bone disease or disorder, bone marrow disease or disorder, condition associated with bone, condition associated with bone marrow, bone fracture, bone defect, cancer, leukemia, lymphoma, myeloma, other blood cancers, myeloma, sickle cell disease, inflammatory disease or disorder, and any combination thereof.

Embodiment 52 provides a method of inducing a bone regeneration in a subject in need thereof, the method comprising administering a therapeutically effectively amount of at least one LNP of any one of Embodiments 1-31 and/or the composition of Embodiment 32 or 33 the same to the subject.

Embodiment 53 provides the method of Embodiment 52, wherein the subject has at least one selected from the group consisting of bone fracture, bone defect, bone cavity, root canal, and nonunion fracture.

Embodiment 54 provides a method of replacing at least one protein in a subject in need thereof, the method comprising administering a therapeutically effectively amount of at least one LNP of any one of Embodiments 1-31 and/or the composition of Embodiment 32 or 33 the same to the subject.

Embodiment 55 provides a method of gene editing in a subject in need thereof, the method comprising administering a therapeutically effectively amount of at least one LNP of any one of Embodiments 1-31 and/or the composition of Embodiment 32 or 33 the same to the subject.

Embodiment 56 provides a method of inducing an immune response in a subject in need thereof, the method comprising administering a therapeutically effectively amount of at least one LNP any one of Embodiments 1-31 and/or the composition of Embodiment 32 or 33 the same to the subject.

Embodiment 57 provides a compound having the structure of Formula (I), or a racemate, enantiomer, diastereomer, pharmaceutically acceptable salt, solvate, or derivative thereof: (A¹)ₓ(L)_{y}(A²)_{z} (I), wherein:
each occurrence of A¹ is independently

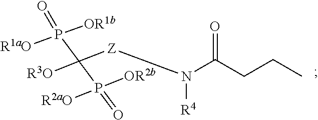

each occurrence of A² is independently

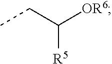

each occurrence of L is an amine linker independently selected from the group consisting of aminoalkyl linker, substituted aminoalkyl linker, diaminoalkyl linker, substituted diaminoalkyl linker, triaminoalkyl linker, substituted triaminoalkyl linker, tetraaminoalkyl linker, substituted tetraaminoalkyl linker, pentaaminoalkyl linker, substituted pentaaminoalkyl linker, polyaminoalkyl linker, substituted polyaminoalkyl linker, aminocycloalkyl linker, substituted aminocycloalkyl linker, diaminocycloalkyl linker, substituted diaminocycloalkyl linker, triaminocycloalkyl linker, substituted triaminocycloalkyl linker, tetraaminocycloalkyl linker, substituted tetraaminocycloalkyl linker, pentaaminocycloalkyl linker, substituted pentaaminocycloalkyl linker, polyaminocycloalkyl linker, substituted poly aminocycloalkyl linker, and any combination thereof;
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1l}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z′}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si;
each occurrence of z′ and z″ is independently an integer represented by 0, 1, or 2;
wherein x, y, and z are independently an integer from 0 to 20;
each occurrence of n is independently an integer from 0 to 10.

Embodiment 58 provides the compound of Embodiment 57, wherein L is selected from the group consisting of

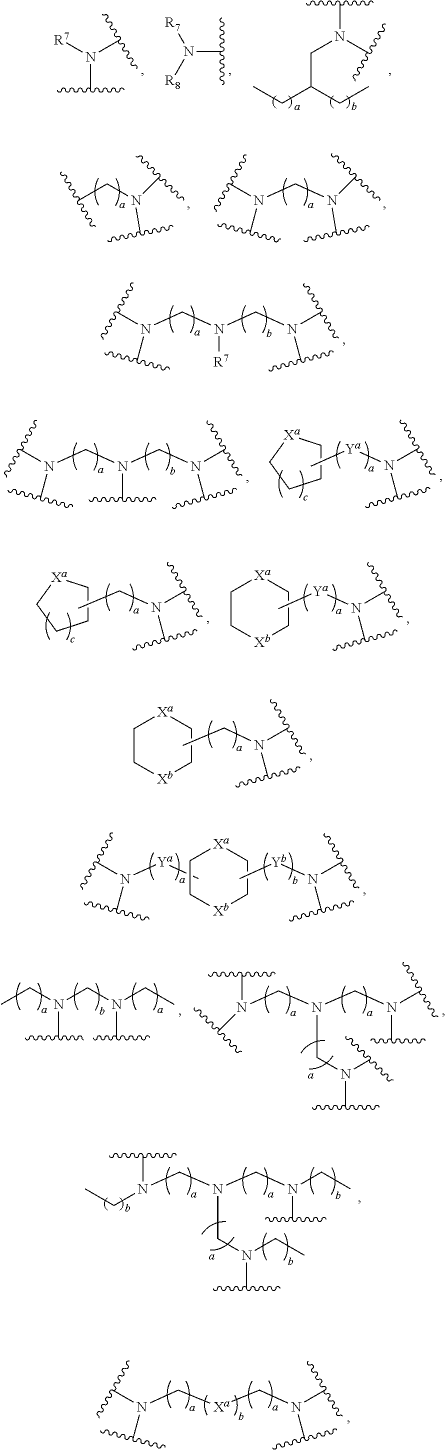

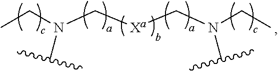

and any combination thereof,
wherein:
each occurrence of R⁷ and R⁸ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aninoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of X^{a} and X^{b} is independently selected from the group consisting of O, S, N(R⁹)_{z′}, P(R⁹)_{z′}, and any combination thereof;
each occurrence of Y^{a} and Y^{b} is independently selected from the group consisting of C₁-C₁₂ alkylenyl, substituted C₁-C₁₂ alkylenyl, C₃-C₈ cycloalkylenyl, substituted C₃-C₈cycloalkylenyl, —Y′(R⁹)_{z′}(R¹⁰)_{z″}—(C₃-C₈ cycloalkylenyl), substituted —Y′(R⁹)_{z′}(R¹⁰)_{z″}—C₃-C₈ cycloalkylenyl, C₂-C₈ heterocycloalkylenyl, substituted C₂-C₈ heterocycloalkylenyl, —Y′(R⁹)_{z′}(R¹⁰)_{z″}—C₂-C₈ heterocycloalkylenyl, substituted —Y′(R⁹)_{z′}(R¹⁰)_{z″}—C₂-C₈ heterocycloalkylenyl, C₂-C₈ alkenylenyl, substituted C₂-C₈ alkenylenyl, C₅-C₁₀ cycloalkenylenyl, substituted C₅-C₁₀ cycloalkenylenyl, —Y′(R⁹)_{z′}(R¹⁰)_{z″}—C₆-C₁₀ cycloalkenylenyl, substituted —Y′(R⁹)_{z′}(R¹⁰)_{z″}—C₅-C₁₀ cycloalkenylenyl, C₂-C₈ alkynylenyl, substituted C₂-C₈ alkynylenyl, C₈-C₁₂ cycloalkynylenyl, substituted C₅-C₁₂ cycloalkynylenyl, —Y′(R⁹)_{z′}(R¹⁰)_{z″}—C₈-C₁₂ cycloalkynylenyl, substituted —Y′(R⁹)_{z′}(R¹⁰)_{z″}—C₈-C₁₂ cycloalkynylenyl, C₆-C₁₀ arylenyl, substituted C₆-C₁₀ arylenyl, —Y′(R⁹)_{z′}(R¹⁰)_{z″}—C₆-C₁₀ arylenyl, substituted —Y′(R⁹)_{z′}(R¹⁰)_{z″}—C₆-C₁₀ arylenyl, C₂-C₁₀ heteroarylenyl, substituted C₂-C₁₀ heteroarylenyl, —Y′(R⁹)_{z′}(R¹⁰)_{z″}—C₂-C₁₀ heteroarylenyl, and substituted —Y′(R⁹)_{z′}(R¹⁰)_{z″}—C₂-C₁₀ heteroarylenyl;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2;
wherein each occurrence of a, b, and c is independently an integer from 0 to 10;
each occurrence of

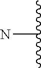 [SMILES: *N]

indicates a bond between a N atom of L and A¹ or A².

Embodiment 59 provides the compound of Embodiment 57 or 58, wherein L is selected from the group consisting of

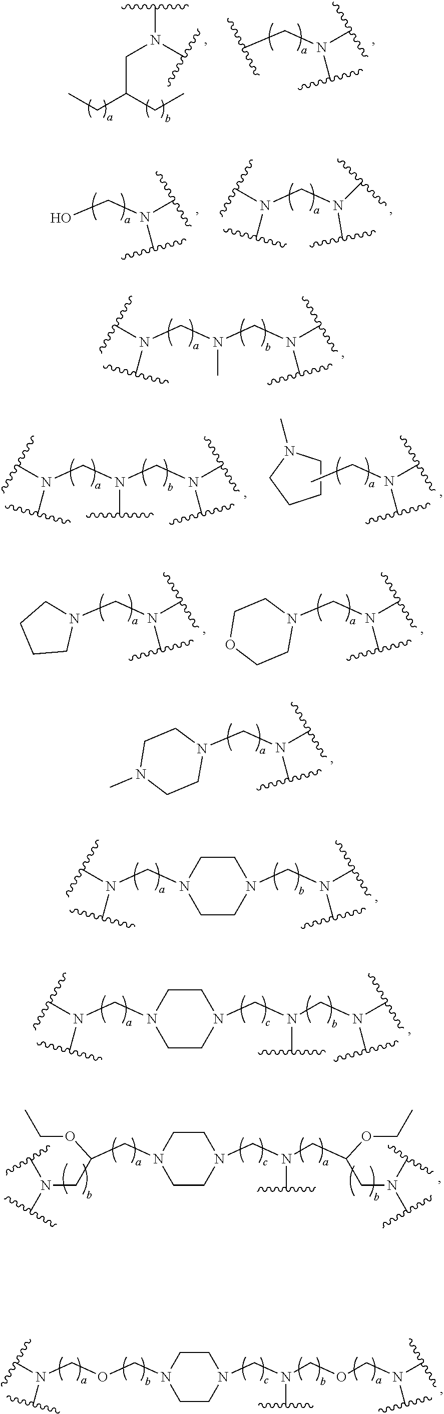

any combination thereof,
wherein:
each occurrence of a b, and c is independently an integer from 0 to 10; and
each occurrence of

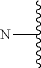 [SMILES: *N]

indicates a bond between a N atom of L and A¹ or A².

Embodiment 60 provides the compound of any one of Embodiments 57-59, wherein the compound having the structure of Formula (I) is a compound having the structure selected from the group consisting of:

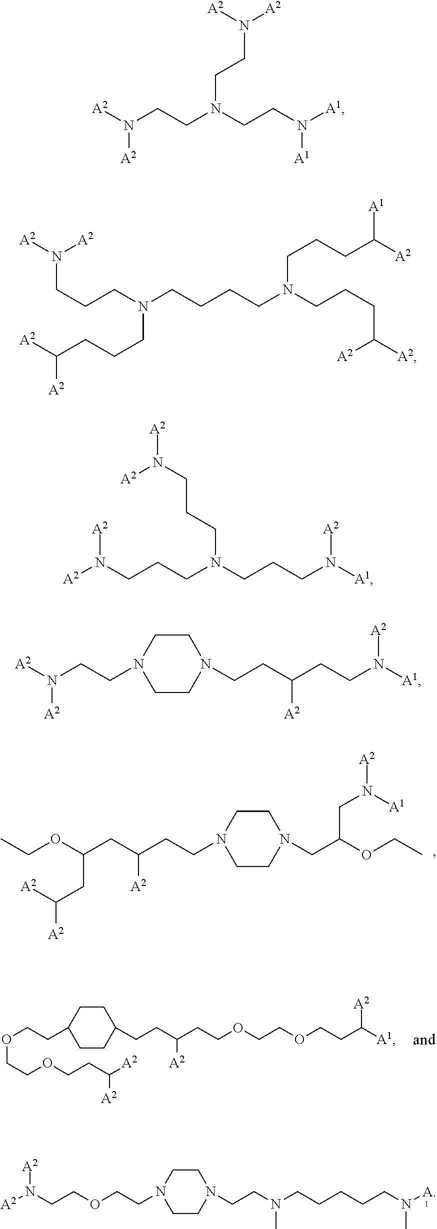

Embodiment 61 provides the compound of Embodiment 60, wherein A¹ is

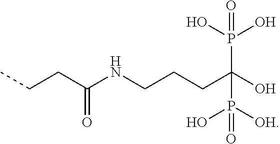

Embodiment 62 provides the compound of Embodiment 60 or 61, wherein each occurrence of A² is independently selected from the group consisting of

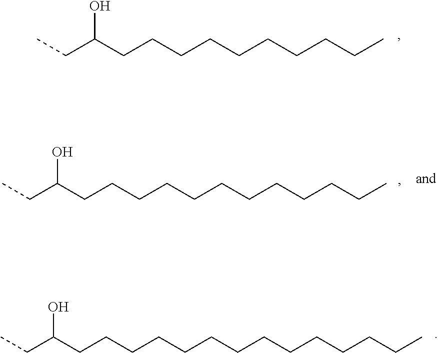

Embodiment 63 provides the compound of any one of Embodiments 57-62, wherein the compound having the structure of Formula (I) is a compound having the structure selected from the group consisting of:

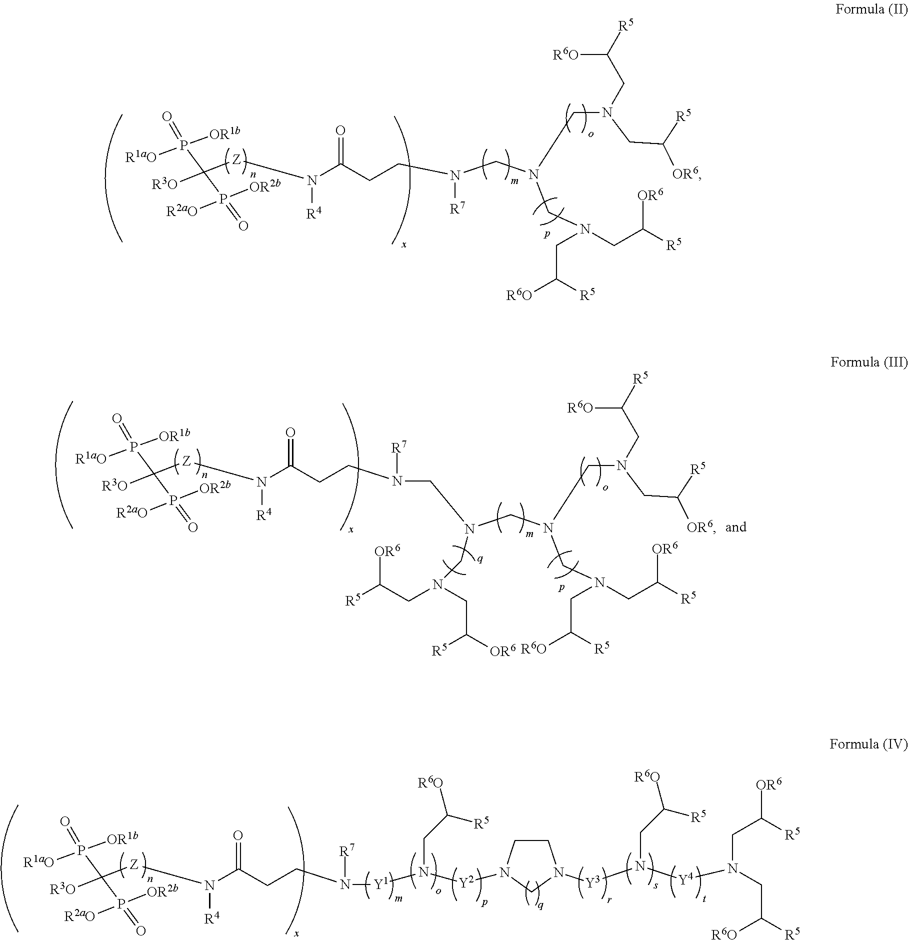

wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof;
each occurrence of Y¹, Y², Y³, and Y⁴ is independently selected from the group consisting of O, S, C(R⁹)_{z′}(R¹⁰)_{z″}, N(R⁹)_{z′}, P(R⁹)_{z′}, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5.

Embodiment 64 provides the compound of any one of Embodiments 57-63, wherein the compound having the structure of Formula (I) is a compound having the structure selected from the group consisting of:

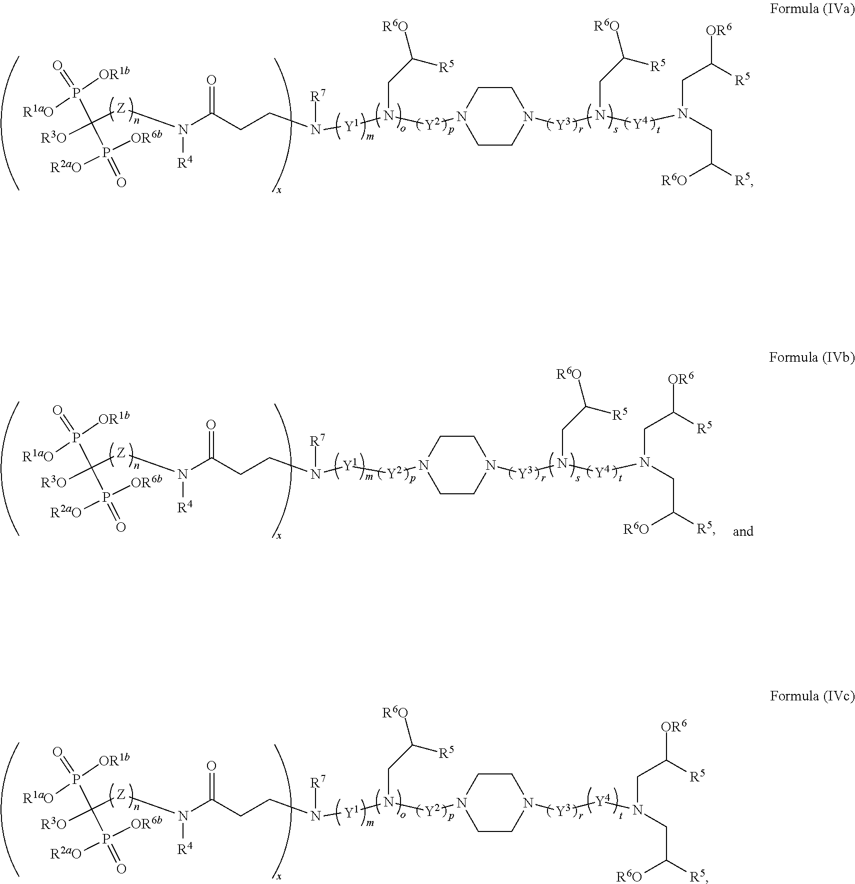

wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl. and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′} (R¹⁰)_{z″}, —NO₂, —CN, ═O, ═S, sulfoxy, sulfonate, sulfate, sulfite, and sulfide, and any combination thereof;
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aninoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof;
each occurrence of Y¹, Y², Y³, and Y⁴ is independently selected from the group consisting of O, S, C(R⁹)_{z′}(R¹⁰)_{z″}, N(R⁹)_{z′}, P(R⁹)_{z′}, and any combination thereof; each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-CLO aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5.

Embodiment 65 provides the compound of any one of Embodiments 57-64, wherein the compound having the structure of Formula (I) is a compound having the structure selected from the group consisting of:

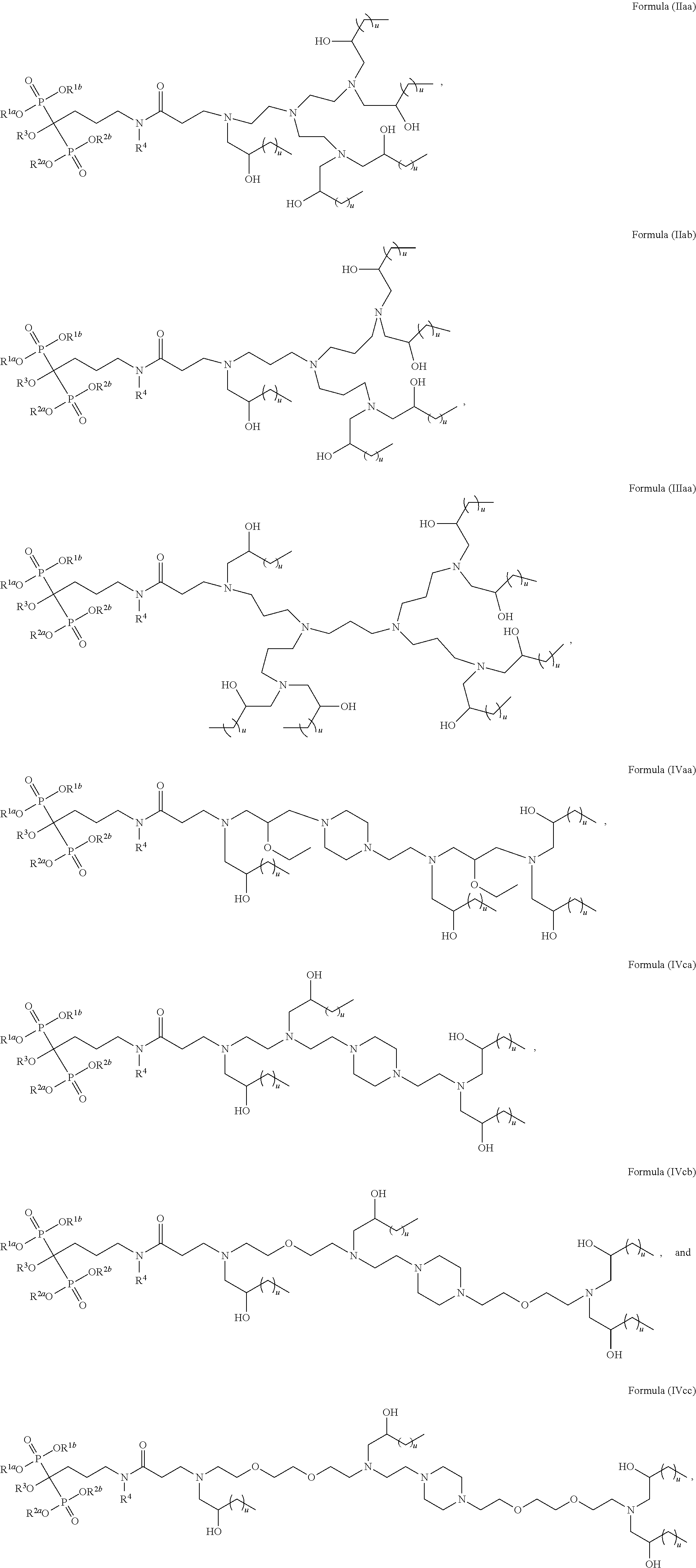

wherein:
each occurrence of R^{1a}, R^{1b}, R^{2a}, R^{2b}, R³, and R⁴ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of R⁹ and R¹⁰ is independently selected from the group consisting of halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein u is an integer from 0 to 20.

Embodiment 66 provides the compound of any one of Embodiments 57-64, wherein the compound having the structure of Formula (I) is selected from the group consisting of

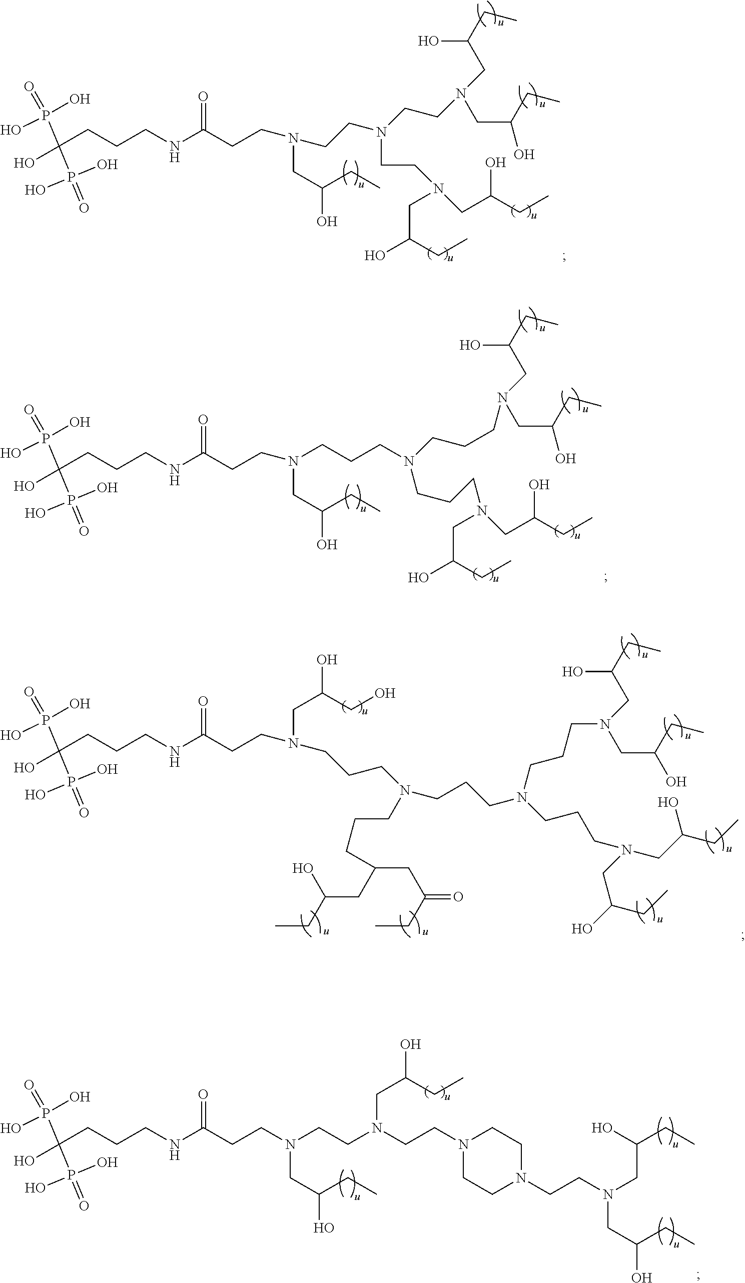

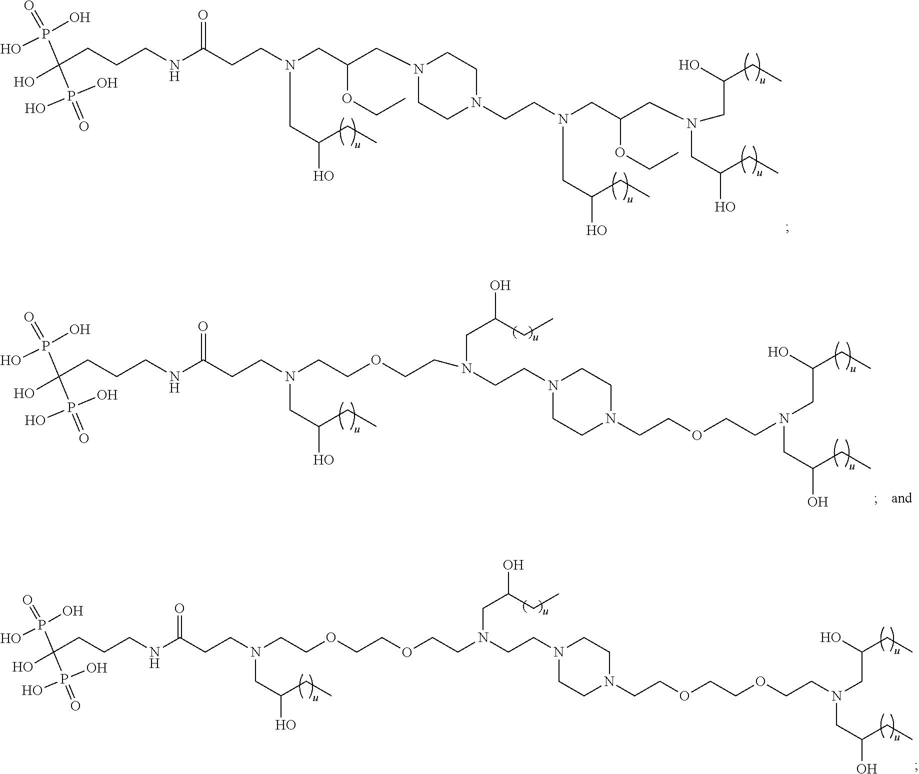

wherein u is an integer from 5 to 15.

Embodiment 67 provides the compound of any one of Embodiments 57-66, wherein the compound having the structure of Formula (I) is.

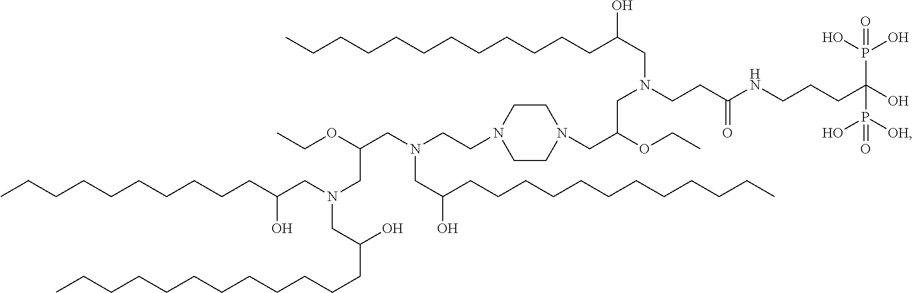 [SMILES: CCCCCCCCCCCCC(O)CN(CCC(=O)NCCCC(O)(P(=O)(O)O)P(=O)(O)O)CC(CN1CCN(CCN(CC(O)CCCCCCCCCCCC)CC(CN(CC(O)CCCCCCCCCC)CC(O)CCCCCCCCCCCC)OCC)CC1)OCC]

(4-(3-((3-(4-(2-((3-(bis(2-hydroxytetradecyl)amino)-2-ethoxypropyl)(2-hydroxytetradecyl)amino)ethyl)piperazin-1-yl)-2-ethoxypropyl)(2-hydroxytetradecyl)amino)propanamido)-1-hydroxybutane-1,1-diyl)bis(phosphonic acid)

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A lipid nanoparticle (LNP) comprising:
(a) at least one compound having the structure of Formula (I), or a racemate, enantiomer, diastereomer, pharmaceutically acceptable salt, solvate, or a derivative thereof: 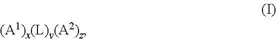
wherein:
each occurrence of A¹ is independently 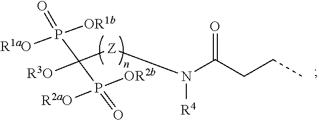
each occurrence of A² is independently 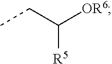
each occurrence of L is an amine linker independently selected from the group consisting of aminoalkyl linker, substituted aminoalkyl linker, diaminoalkyl linker, substituted diaminoalkyl linker, triaminoalkyl linker, substituted triaminoalkyl linker, tetraaminoalkyl linker, substituted tetraaminoalkyl linker, pentaaminoalkyl linker, substituted pentaaminoalkyl linker, polyaminoalkyl linker, substituted polyaminoalkyl linker, aminocycloalkyl linker, substituted aminocycloalkyl linker, diaminocycloalkyl linker, substituted diaminocycloalkyl linker, triaminocycloalkyl linker, substituted triaminocycloalkyl linker, tetraaminocycloalkyl linker, substituted tetraaminocycloalkyl linker, pentaaminocycloalkyl linker, substituted pentaaminocycloalkyl linker, polyaminocycloalkyl linker, substituted polyaminocycloalkyl linker, and any combination thereof;
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si;
each occurrence of z′ and z″ is independently an integer represented by 0, 1, or 2;
wherein x, y, and z are independently an integer from 0 to 20;
each occurrence of n is independently an integer from 0 to 10; and
the compound is present in a concentration range of about 1 mol % to about 99 mol %;
(b) at least one neutral phospholipid, wherein the neutral phospholipid is present in a concentration range of about 5 mol % to about 45 mol %;
(c) at least one cholesterol lipid, wherein the total cholesterol lipid is in a concentration range of about 5 mol % to about 55 mol %; and
(d) at least one polymer conjugated lipid (e.g., polyethylene glycol (PEG)-conjugated lipid), wherein the total polymer conjugated lipid is present in a concentration range of about 0.5 mol % to about 12.5 mol %.

2. The LNP of claim 1, wherein at least one of the following applies:
(a) L is selected from the group consisting of 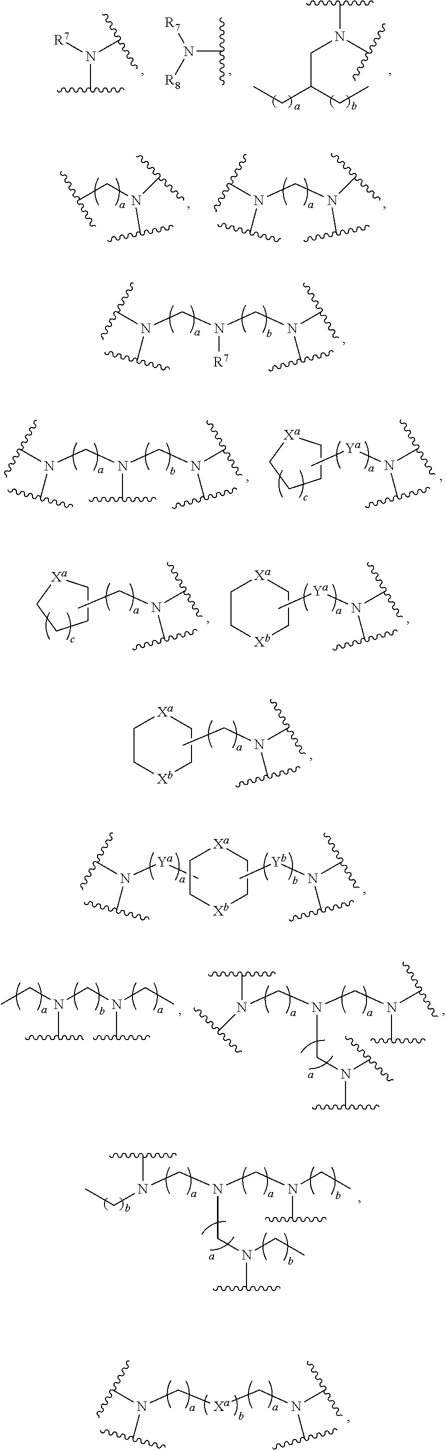 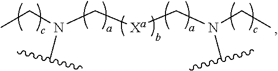
and any combination thereof,
wherein:
each occurrence of R⁷ and R⁸ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of X^{a} and X^{b} is independently selected from the group consisting of O, S, N(R⁹)_{z′}, P(R⁹)_{z′}, and any combination thereof;
each occurrence of Y^{a} and Y^{b} is independently selected from the group consisting of C₁-C₁₂ alkylenyl, substituted C₁-C₁₂ alkylenyl, C₃-C₈ cycloalkylenyl, substituted C₃-C₈ cycloalkylenyl, —Y′(R⁹)_{z′}(R¹⁰)_{z″}—(C₃-C₈ cycloalkylenyl), substituted —Y′(R⁹)_{z′}(R¹⁰)_{z″}—C₃-C₈ cycloalkylenyl, C₂-C₈ heterocycloalkylenyl, substituted C₂-C₈ heterocycloalkylenyl, —Y′(R⁹)_{z′}(R¹⁰)_{z″}—C₂-C₈ heterocycloalkylenyl, substituted-Y′(R⁹)_{z′}(R¹⁰)_{z″}—C₂-C₈ heterocycloalkylenyl, C₂-C₈ alkenylenyl, substituted C₂-C₈ alkenylenyl, C₅-C₁₀ cycloalkenylenyl, substituted C₅-C₁₀ cycloalkenylenyl, —Y′(R⁹)_{z′}(R¹⁰)_{z″}—C₅-C₁₀ cycloalkenylenyl, substituted —Y′(R⁹)_{z′}(R¹⁰)_{z″}—C₅-C₁₀ cycloalkenylenyl, C₂-C₈ alkynylenyl, substituted C₂-C₈ alkynylenyl, C₈-C₁₂ cycloalkynylenyl, substituted C₈-C₁₂ cycloalkynylenyl, —Y′(R⁹)_{z′}(R¹⁰)_{z″}—C₈-C₁₂ cycloalkynylenyl, substituted —Y′(R⁹)_{z′}(R¹⁰)_{z″}—C₈-C₁₂ cycloalkynylenyl, C₆-C₁₀ arylenyl, substituted C₆-C₁₀ arylenyl, —Y′(R⁹)_{z′}(R¹⁰)_{z″}—C₆-C₁₀ arylenyl, substituted —Y′(R⁹)_{z′}(R¹⁰)_{z″}—C₆-C₁₀ arylenyl, C₂-C₁₀ heteroarylenyl, substituted C₂-C₁₀ heteroarylenyl, —Y′(R⁹)_{z′}(R¹⁰)_{z″}—C₂-C₁₀ heteroarylenyl, and substituted —Y′(R⁹)_{z′}(R¹⁰)_{z″}—C₂-C₁₀ heteroarylenyl;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2;
wherein each occurrence of a, b, and c is independently an integer from 0 to 10;
each occurrence of 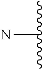 [SMILES: *N]
indicates a bond between a N atom of L and A¹ or A²; and
(b) L is selected from the group consisting of: 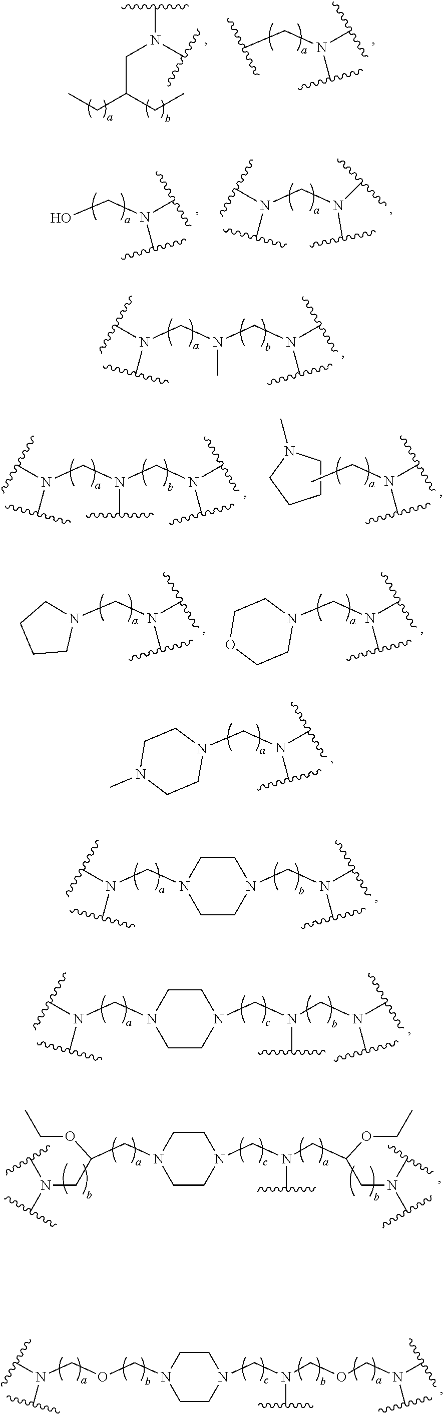
and any combination thereof,
wherein:
each occurrence of a b, and c is independently an integer from 0 to 10; and
each occurrence of 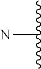 [SMILES: *N]
indicates a bond between a N atom of L and A¹ or A².

3. (canceled)

4. The LNP of claim 1, wherein at least one of the following applies:
(a) the compound having the structure of Formula (I) is a compound having the structure selected from the group consisting of: 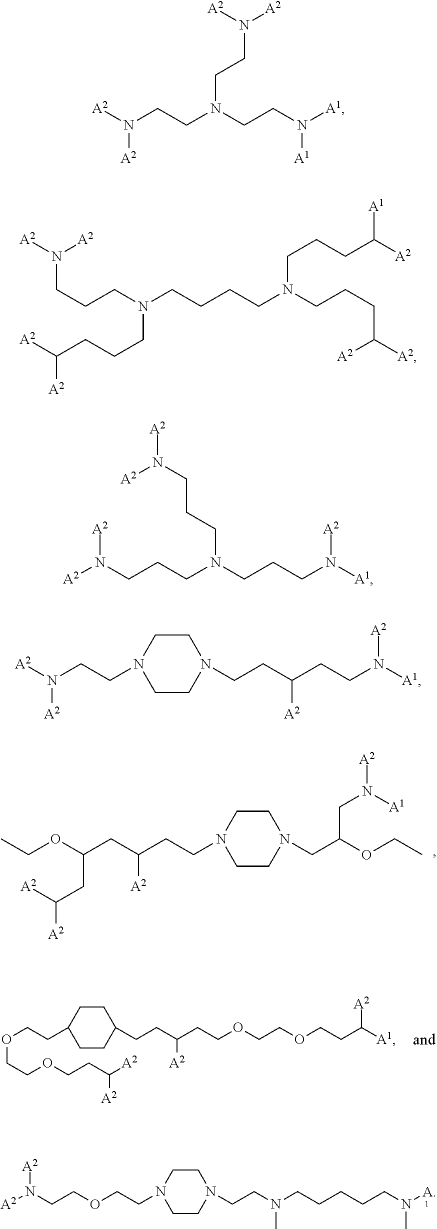
and
(b) the compound having the structure of Formula (I) is a compound having the structure selected from the group consisting of: 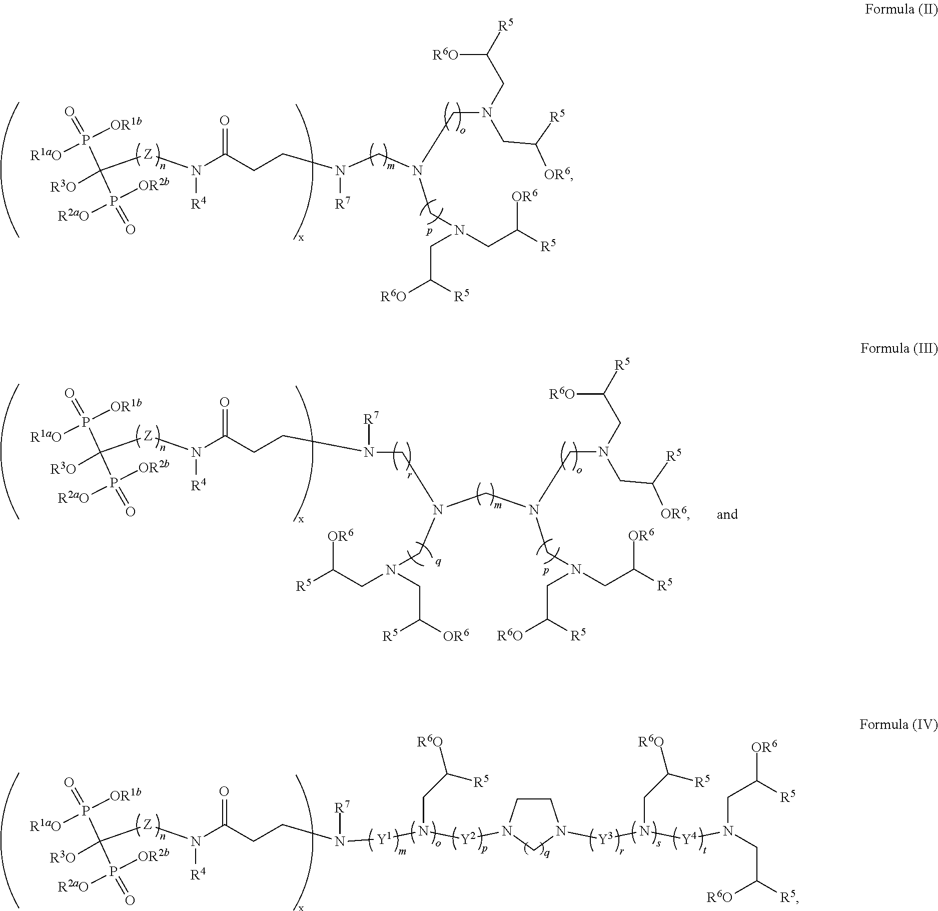
wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof;
each occurrence of Y¹, Y², Y³, and Y⁴ is independently selected from the group consisting of O, S, C(R⁹)_{z′}(R¹⁰)_{z″}, N(R⁹)_{z′}, P(R⁹)_{z′}, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5.

5. The LNP of claim 4, wherein at least one of the following applies:
(a) A¹ is 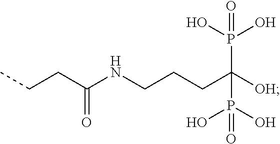
and
(b) each occurrence of A² is independently selected from the group consisting of

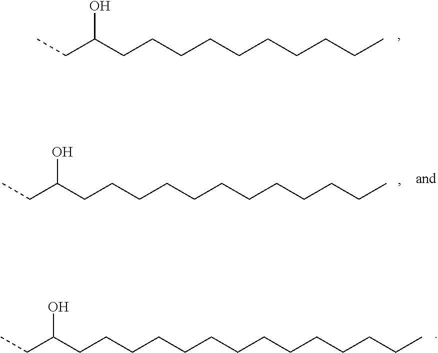

6-7. (canceled)

8. The LNP of claim 1, wherein the compound having the structure of Formula (I) is a compound having the structure selected from the group consisting of: 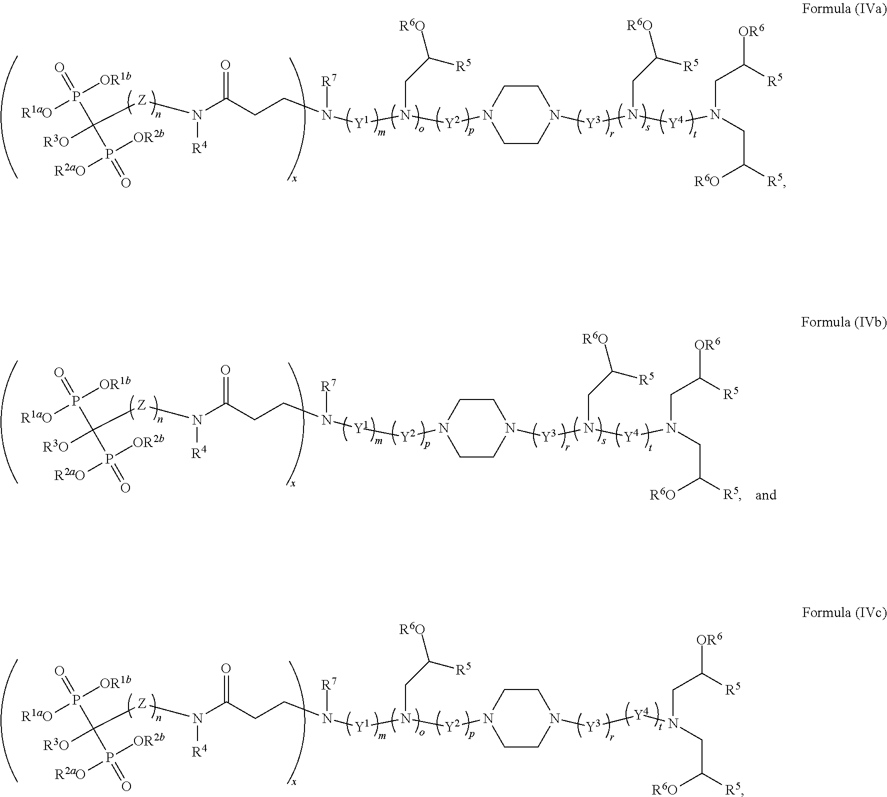
wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof;
each occurrence of Y¹, Y², Y³, and Y⁴ is independently selected from the group consisting of O, S, C(R⁹)_{z′}(R¹⁰)_{z″}, N(R⁹)_{z′}, P(R⁹)_{z′}, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently 0, 1, or 2;
wherein x is an integer from 0 to 20;
wherein m, o, p, q, r, s, and t are independently an integer from 0 to 10;
each occurrence of n is independently an integer from 0 to 5, and
each occurrence of u is independently an integer from 5 to 15;
optionally wherein at least one of the following applies:
(a) the compound having the structure of Formula (I) is a compound having the structure selected from the group consisting of: 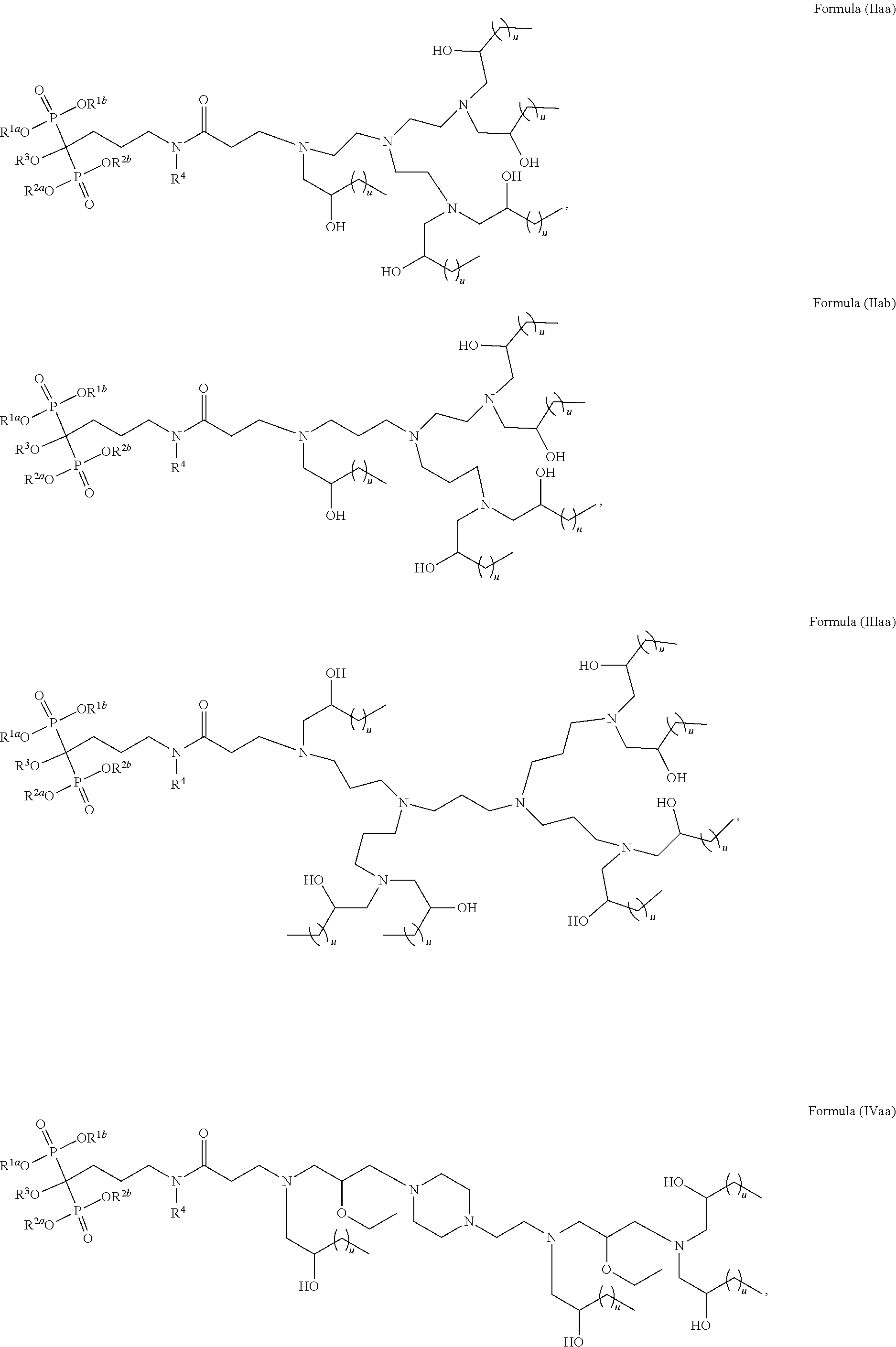 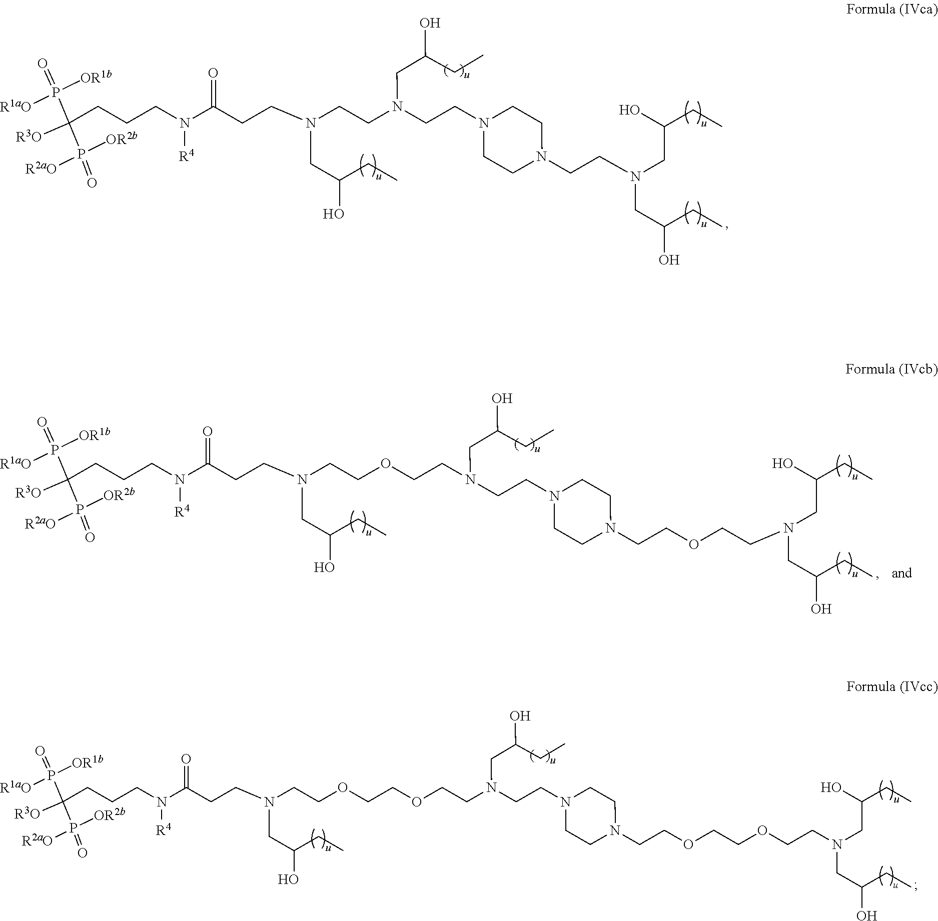
and
(b) the compound having the structure of Formula (I) is selected from the group consisting of:

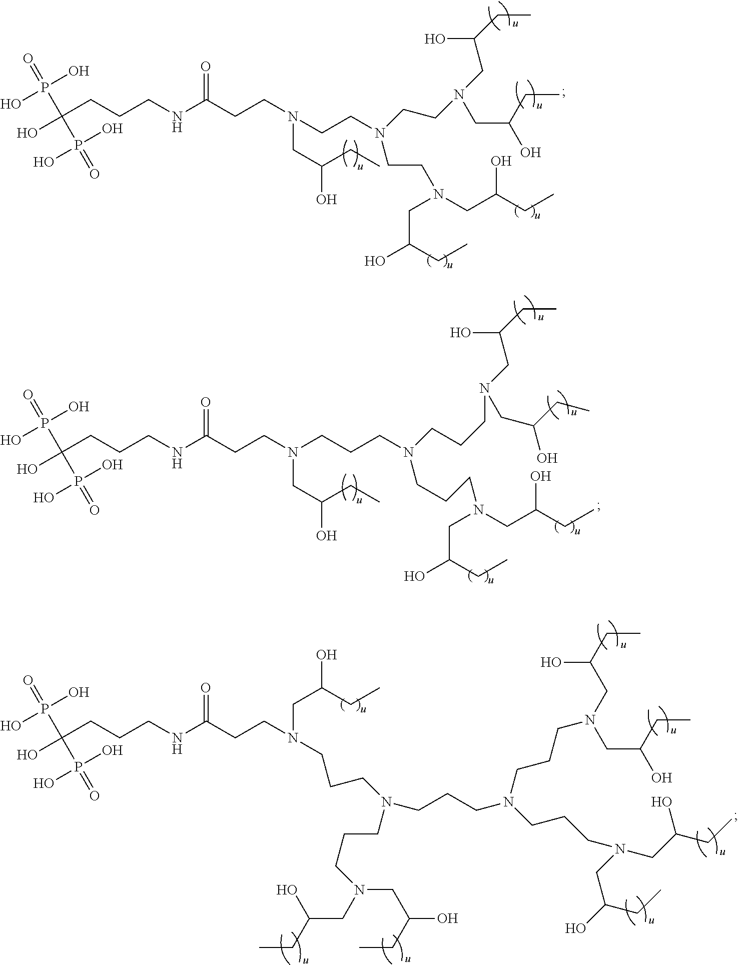

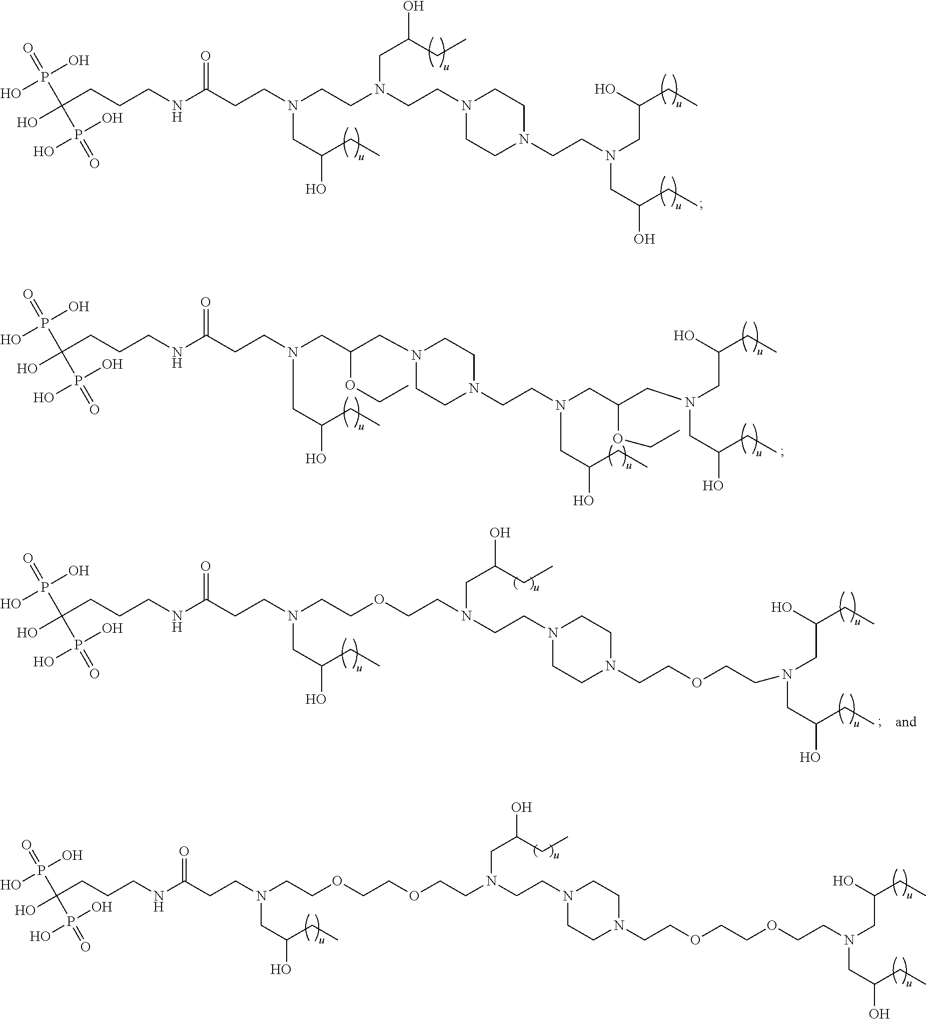

9-10. (canceled)

11. The LNP of claim 1, wherein at least one of the following applies:
(a) the compound having the structure of Formula (I) is: 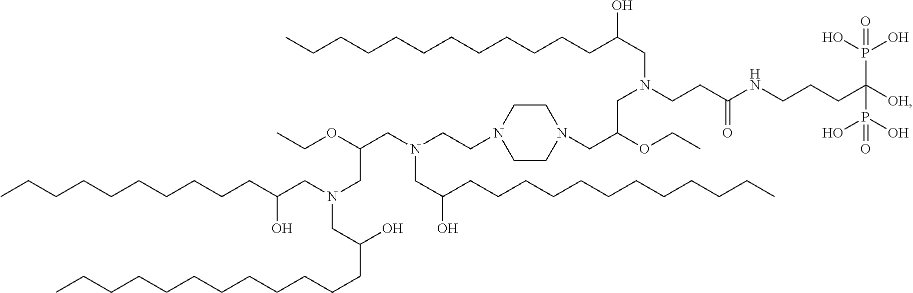 [SMILES: CCCCCCCCCCCCC(O)CN(CCC(=O)NCCCC(O)(P(=O)(O)O)P(=O)(O)O)CC(CN1CCN(CCN(CC(O)CCCCCCCCCCCC)CC(CN(CC(O)CCCCCCCCCC)CC(O)CCCCCCCCCCCC)OCC)CC1)OCC]
(4-(3-((3-(4-(2-((3-(bis(2-hydroxytetradecyl)amino)-2-ethoxypropyl)(2-hydroxytetradecyl)amino)ethyl)piperazin-1-yl)-2-ethoxypropyl)(2-hydroxytetradecyl)amino)propanamido)-1-hydroxybutane-1,1-diyl)bis(phosphonic acid),
(b) the at least one neutral phospholipid comprises at least one selected from the group consisting of dioleoyl-phosphatidylethanolamine (DOPE), dioleoylphosphatidylcholine (DOPC), distearoylphosphatidylcholine (DSPC), distearoyl-phosphatidylethanolamine (DSPE), 16-O-dimethyl PE, 18-1-trans PE, 1-stearoyl-2-oleoyl-phosphatidyethanol amine (SOPE), stearoyloleoylphosphatidylcholine (SOPC), and 2-dioleoyl-3-trimethylammonium-propane (DOTAP), or any combination thereof;
(c) the at least one cholesterol lipid comprises at least one selected from the group consisting of a cholesterol and a cholesteryl derivate, or any combination thereof; and
(d) the at least one polymer conjugated lipid is a PEG conjugated lipid, optionally wherein the PEG-conjugated lipid comprises at least one selected from the group consisting of 1,2-dimyristoyl-sn-glycero-3-phosphoethanolamine-N-[methoxy(polyethylene glycol)-2000](C14-PEG2000), C12-PEG2000, C12-PEG490, 1,2-dimyristoyl-rac-glycero-3-methoxypolyethylene glycol-2000 (DMG-PEG2000), 1,2-distearoyl-sn-glycero-3-phosphoethanolamine-N-[amino(polyethylene glycol)-2000] (DSPE-PEG 2000 amine), and any combination thereof.

12-15. (canceled)

16. The LNP of claim 1, wherein at least one of the following applies:
(a) the molar ratio of (a):(b):(c):(d) is about 1-80%:5-45%:5-55%:0.5-12.5%, optionally wherein the molar ratio of (a):(b):(c):(d) is about 35-45%:5-20%:40-55%:1-2.5%, and optionally wherein the molar ratio of (a):(b):(c):(d) is about 30-35%:16%:46.5%:2.5%; and
(b) the LNP has a diameter of between about 10 nm to about 1000 nm, optionally wherein the LNP has a diameter of between about 50 nm to about 500 nm;
(c) the LNP selectively binds to at least one target cell of interest, optionally wherein the at least one target cell of interest is selected from the group consisting of a stem cell, bone cell, bone marrow cell, and any combination thereof, and optionally wherein the at least one target cell of interest is selected from the group consisting of a stem cell, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, and any combination thereof; and
(d) the LNP further comprises or encapsulates at least one agent.

17-24. (canceled)

25. The LNP of claim 16, wherein at least one of the following applies:
(a) the weight ratio of (a): the at least one agent is between about 1:1 to about 10:1;
(b) the at least one agent is selected from the group consisting of a nucleic acid molecule, a small molecule, a protein, an antibody, and any combination thereof, optionally wherein the nucleic acid molecule is a DNA molecule or an RNA molecule, and optionally wherein the nucleic acid molecule is selected from the group consisting of cDNA, mRNA, miRNA, siRNA, sgRNA, modified RNA, CRISPR-Cas9, antagomir, antisense molecule, targeted nucleic acid, and any combination thereof;
(c) the LNP has a diameter of between about 10 nm to about 1000 nm, optionally wherein the LNP has a diameter of between about 50 nm to about 500 nm; and
(d) the LNP is suitable for delivering a nucleic acid to a bone cell, bone marrow cell, or a combination thereof.

26-31. (canceled)

32. A composition comprising at least one LNP of claim 1, optionally further comprising at least one pharmaceutically acceptable excipient, optionally wherein the composition is suitable for delivering a nucleic acid to a bone cell, bone marrow cell, or a combination thereof.

33. (canceled)

34. A method of delivering an agent to a subject in need thereof, the method comprising administering a therapeutically effectively amount of at least one LNP ofany claim 1 and/or a pharmaceutical composition thereof to the subject.

35. The method of claim 34, wherein at least one of the following applies:
(a) the agent is selected from the group consisting of a nucleic acid molecule, a small molecule, a protein, an antibody, a therapeutic agent, and any combination thereof, optionally wherein the nucleic acid molecule is a DNA molecule or an RNA molecule, optionally wherein the nucleic acid molecule is selected from the group consisting of cDNA, mRNA, miRNA, siRNA, sgRNA, modified RNA, CRISPR-Cas9, antagomir, antisense molecule, targeted nucleic acid, and any combination thereof, optionally wherein the LNP selectively binds to at least one target cell of interest, and optionally wherein the at least one target cell of interest is selected from the group consisting of a stem cell, bone cell, bone marrow cell, and any combination thereof; and
(b) the agent is encapsulated within the LNP, optionally wherein the LNP or the composition comprising the same is administered intravenously, intramuscularly, intradermally, subcutaneously, intranasally, by inhalation, or any combination thereof.

36-42. (canceled)

43. A method of delivering an agent to a bone in a subject, bone marrow, or a combination thereof, the method comprising administering a therapeutically effectively amount of at least one LNP of claim 1 and/or a pharmaceutical composition thereof to the subject.

44. The method of claim 43, wherein at least one of the following applies:
(a) the agent is selected from the group consisting of a nucleic acid molecule, a small molecule, a protein, an antibody, a therapeutic agent, and any combination thereof, optionally wherein the nucleic acid molecule is a DNA molecule or an RNA molecule, and optionally wherein the nucleic acid molecule is selected from the group consisting of cDNA, mRNA, miRNA, siRNA, sgRNA, modified RNA, CRISPR-Cas9, antagomir, antisense molecule, targeted nucleic acid, and any combination thereof;
(b) the LNP selectively binds to at least one bone cell, bone marrow cell, or a combination thereof;
(c) the agent is encapsulated within the LNP; and
(d) the LNP or the composition comprising the same is administered intravenously, intramuscularly, intradermally, subcutaneously, intranasally, by inhalation, or any combination thereof.

45-49. (canceled)

50. A method of:
(a) treating, ameliorating, and/or preventing at least one disease, disorder, or condition in a subject in need thereof;
(b) inducing a bone regeneration in a subject in need thereof;
(c) replacing at least one protein in a subject in need thereof;
(d) gene editing in a subject in need thereof; and
(e) inducing an immune response in a subject in need thereof;
the method comprising administering a therapeutically effectively amount of at least one LNP of claim 1 and/or a pharmaceutical composition thereof to the subject.

51-56. (canceled)

57. A compound having the structure of Formula (I), or a racemate, enantiomer, diastereomer, pharmaceutically acceptable salt, solvate, or derivative thereof: 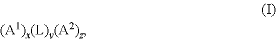
wherein:
each occurrence of A¹ is independently 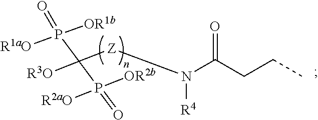
each occurrence of A² is independently 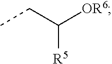
each occurrence of L is an amine linker independently selected from the group consisting of aminoalkyl linker, substituted aminoalkyl linker, diaminoalkyl linker, substituted diaminoalkyl linker, triaminoalkyl linker, substituted triaminoalkyl linker, tetraaminoalkyl linker, substituted tetraaminoalkyl linker, pentaaminoalkyl linker, substituted pentaaminoalkyl linker, polyaminoalkyl linker, substituted polyaminoalkyl linker, aminocycloalkyl linker, substituted aminocycloalkyl linker, diaminocycloalkyl linker, substituted diaminocycloalkyl linker, triaminocycloalkyl linker, substituted triaminocycloalkyl linker, tetraaminocycloalkyl linker, substituted tetraaminocycloalkyl linker, pentaaminocycloalkyl linker, substituted pentaaminocycloalkyl linker, polyaminocycloalkyl linker, substituted polyaminocycloalkyl linker, and any combination thereof;
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si;
each occurrence of z′ and z″ is independently an integer represented by 0, 1, or 2;
wherein x, y, and z are independently an integer from 0 to 20;
each occurrence of n is independently an integer from 0 to 10.

58. The compound of claim 57, wherein at least one of the following applies:
(a) L is selected from the group consisting of: 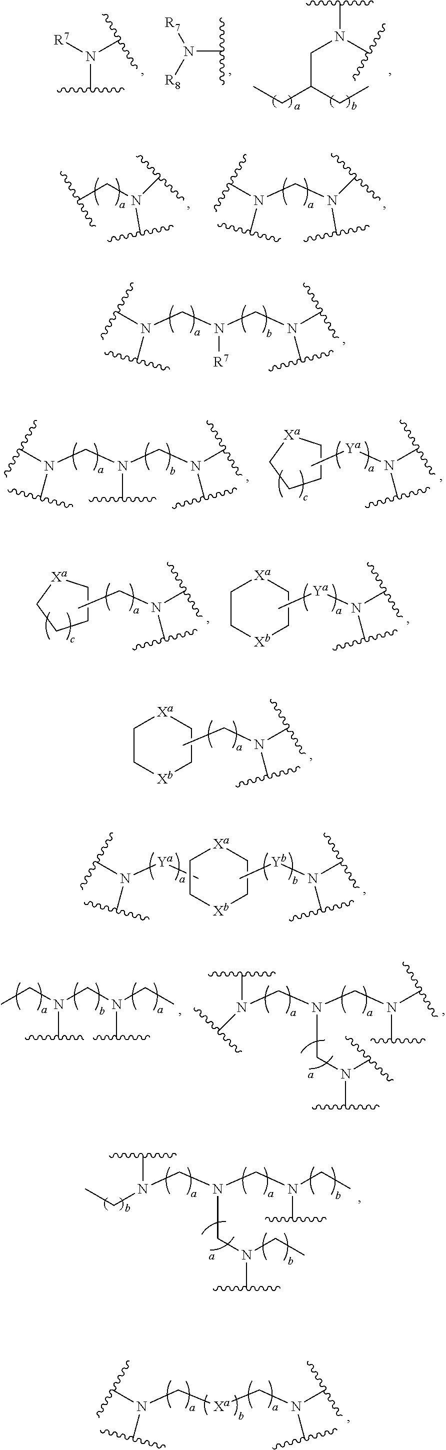 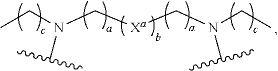
and any combination thereof,
wherein:
each occurrence of R⁷ and R⁸ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of X^{a} and X^{b} is independently selected from the group consisting of O, S, N(R⁹)_{z′}, P(R⁹)_{z′}, and any combination thereof;
each occurrence of Y^{a} and Y^{b} is independently selected from the group consisting of C₁-C₁₂ alkylenyl, substituted C₁-C₁₂ alkylenyl, C₃-C₈ cycloalkylenyl, substituted C₃-C₈ cycloalkylenyl, —Y′(R⁹)_{z′}(R¹⁰)_{z″}—(C₃-C₈ cycloalkylenyl), substituted —Y′(R⁹)_{z′}(R¹⁰)_{z″}—C₃-C₈ cycloalkylenyl, C₂-C₈ heterocycloalkylenyl, substituted C₂-C₈ heterocycloalkylenyl, —Y′(R⁹)_{z′}(R¹⁰)_{z″}—C₂-C₈ heterocycloalkylenyl, substituted-Y′(R⁹)_{z′}(R¹⁰)_{z″}—C₂-C₈ heterocycloalkylenyl, C₂-C₈ alkenylenyl, substituted C₂-C₈ alkenylenyl, C₅-C₁₀ cycloalkenylenyl, substituted C₅-C₁₀ cycloalkenylenyl, —Y′(R⁹)_{z′}(R¹⁰)_{z″}—C₅-C₁₀ cycloalkenylenyl, substituted —Y′(R⁹)_{z′}(R¹⁰)_{z″}—C₅-C₁₀ cycloalkenylenyl, C₂-C₈ alkynylenyl, substituted C₂-C₈ alkynylenyl, C₈-C₁₂ cycloalkynylenyl, substituted C₈-C₁₂ cycloalkynylenyl, —Y′(R⁹)_{z′}(R¹⁰)_{z″}—C₈-C₁₂ cycloalkynylenyl, substituted —Y′(R⁹)_{z′}(R¹⁰)_{z″}—C₈-C₁₂ cycloalkynylenyl, C₆-C₁₀ arylenyl, substituted C₆-C₁₀ arylenyl, —Y′(R⁹)_{z′}(R¹⁰)_{z″}—C₆-C₁₀ arylenyl, substituted —Y′(R⁹)_{z′}(R¹⁰)_{z″}—C₆-C₁₀ arylenyl, C₂-C₁₀ heteroarylenyl, substituted C₂-C₁₀ heteroarylenyl, —Y′(R⁹)_{z′}(R¹⁰)_{z″}—C₂-C₁₀ heteroarylenyl, and substituted —Y′(R⁹)_{z′}(R¹⁰)_{z″}—C₂-C₁₀ heteroarylenyl;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2;
wherein each occurrence of a, b, and c is independently an integer from 0 to 10;
each occurrence of 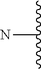 [SMILES: *N]
indicates a bond between a N atom of L and A¹ or A²;
(b) L is selected from the group consisting of: 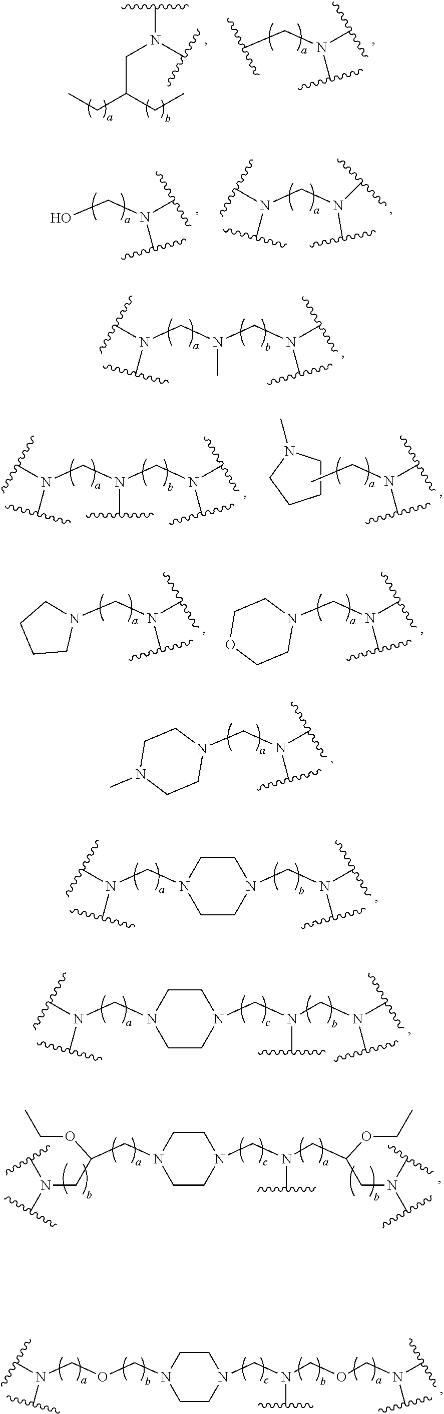
any combination thereof,
wherein:
each occurrence of a, b, and c is independently an integer from 0 to 10; and
each occurrence of 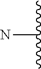 [SMILES: *N]
indicates a bond between a N atom of L and A¹ or A².

59. (canceled)

60. The compound of claim 57, wherein the compound having the structure of Formula (I) is a compound having the structure selected from the group consisting of:

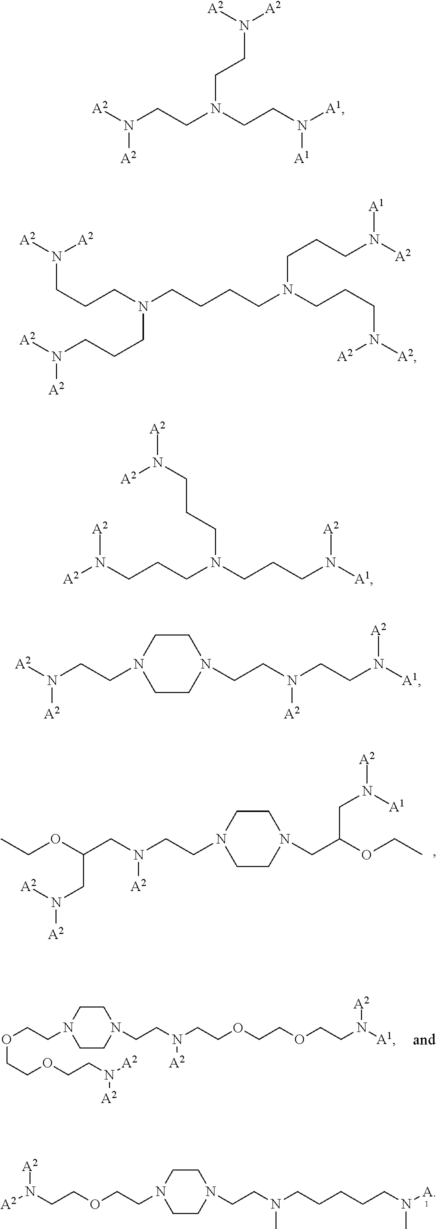

61. The compound of claim 60, wherein at least one of the following applies:
(a) A¹ is 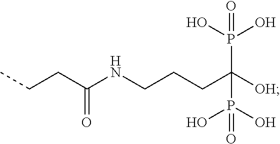
and
(b) each occurrence of A² is independently selected from the group consisting of:

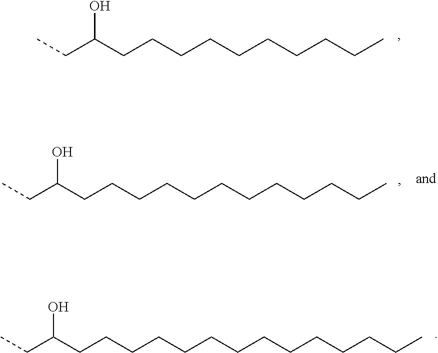

62. (canceled)

63. The compound of claim 57, wherein at least one of the following applies:
(a) the compound having the structure of Formula (I) is a compound having the structure selected from the group consisting of: 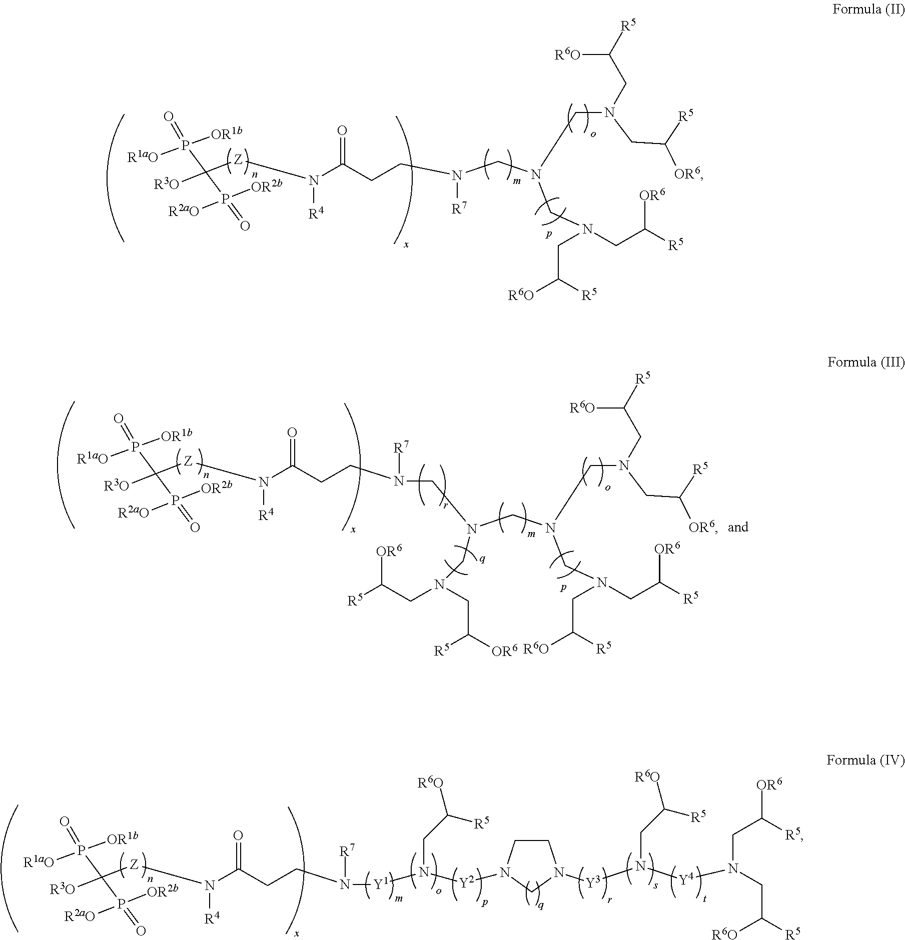
wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₅-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof;
each occurrence of Y¹, Y², Y³, and Y⁴ is independently selected from the group consisting of O, S, C(R⁹)_{z′}(R¹⁰)_{z″}, N(R⁹)_{z′}, P(R⁹)_{z′}, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5;
(b) the compound having the structure of Formula (I) is a compound having the structure selected from the group consisting of: 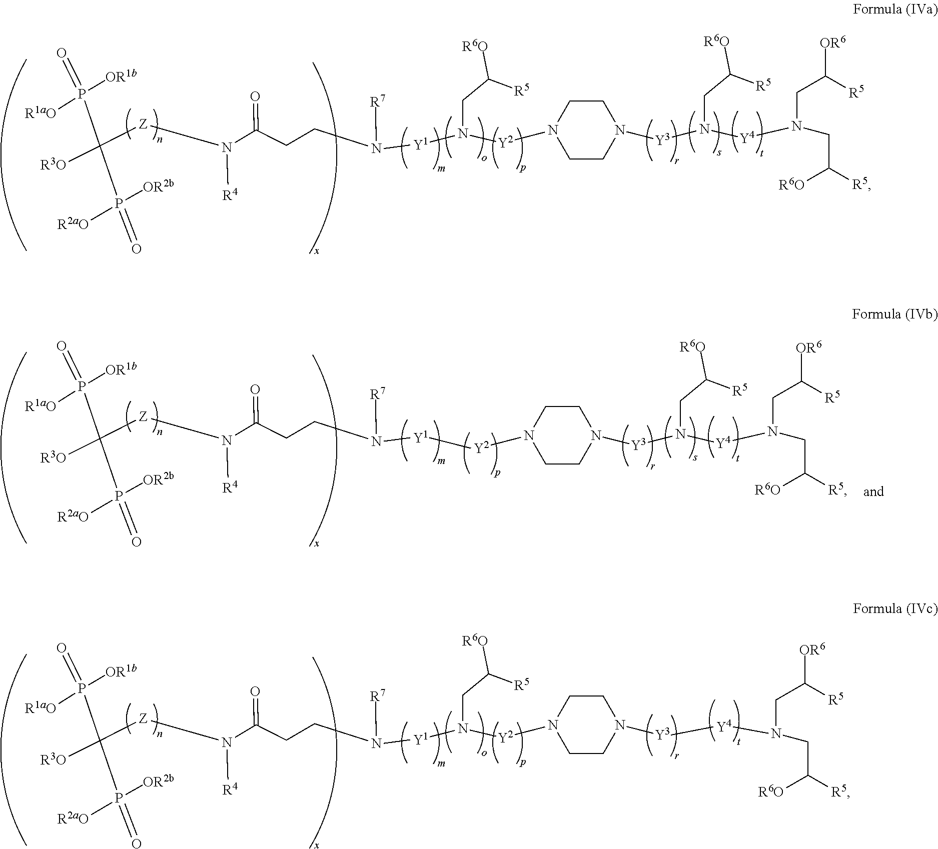
wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{11b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof;
each occurrence of Y¹, Y², Y³, and Y⁴ is independently selected from the group consisting of O, S, C(R⁹)_{z′}(R¹⁰)_{z″}, N(R⁹)_{z′}, P(R⁹)_{z′}, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5; and
(c) the compound having the structure of Formula (I) is a compound having the structure selected from the group consisting of: 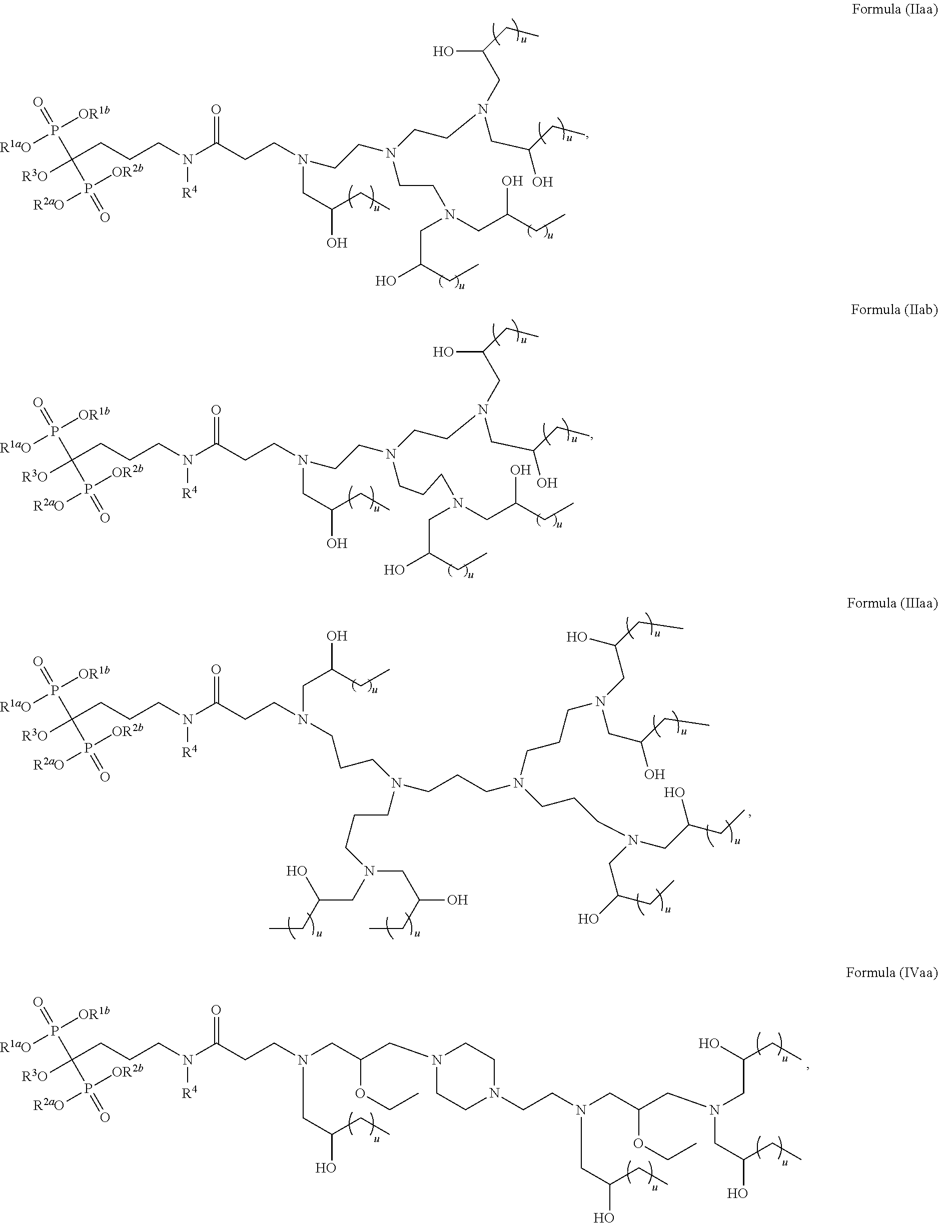 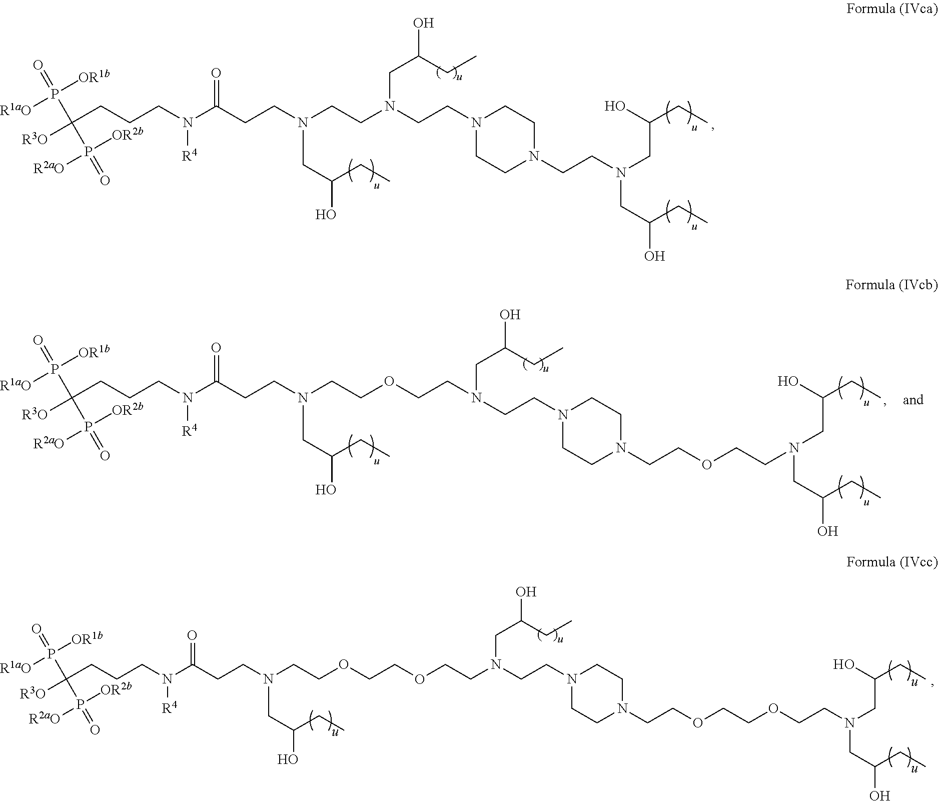
wherein:
each occurrence of R^{1a}, R^{1b}, R^{2a}, R^{2b}, R³, and R⁴ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of R⁹ and R¹⁰ is independently selected from the group consisting of halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein u is an integer from 0 to 20.

64-65. (canceled)

66. The compound of claim 57, wherein at least one of the following applies:
(a) the compound having the structure of Formula (I) is selected from the group consisting of: 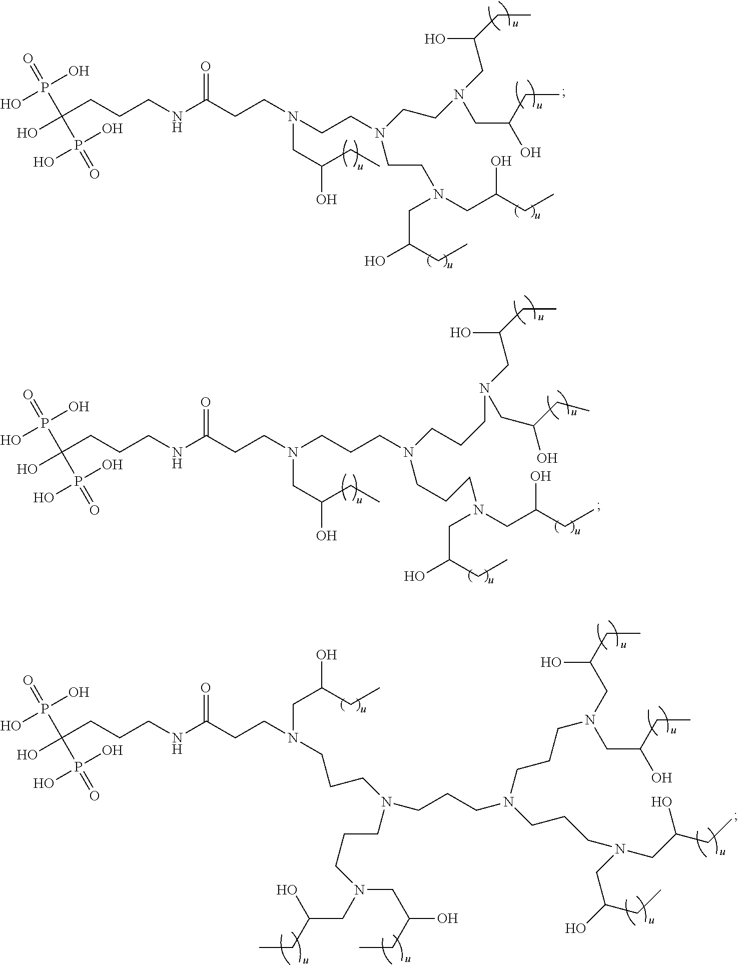 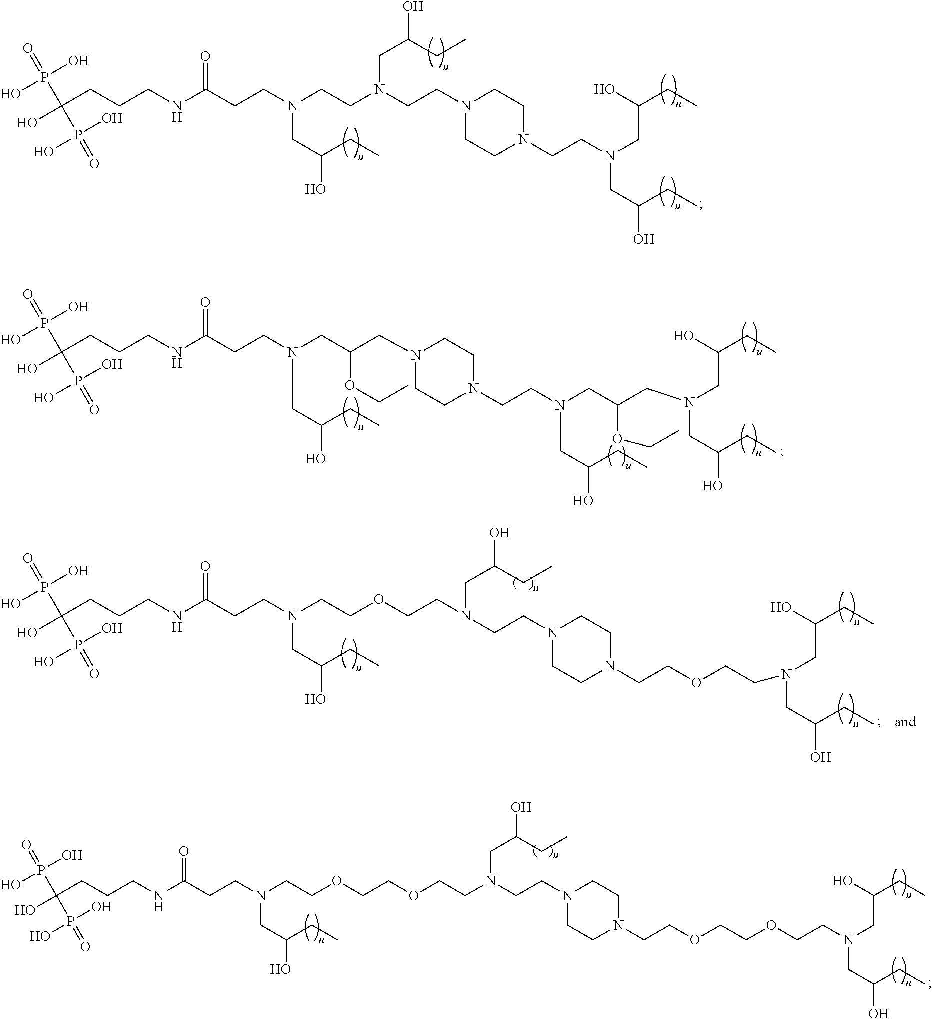
wherein u is an integer from 5 to 15, and
(b) the compound having the structure of formula (I) is: 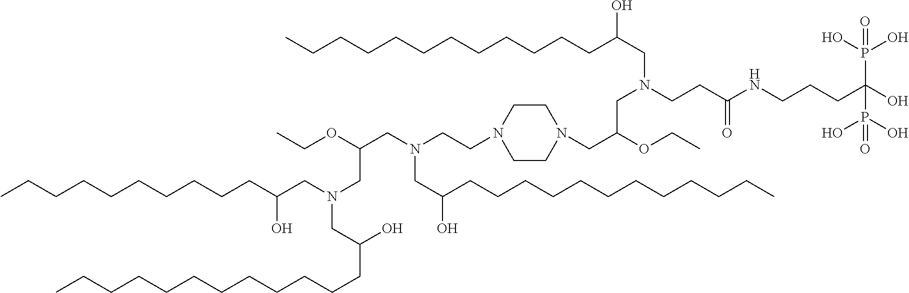 [SMILES: CCCCCCCCCCCCC(O)CN(CCC(=O)NCCCC(O)(P(=O)(O)O)P(=O)(O)O)CC(CN1CCN(CCN(CC(O)CCCCCCCCCCCC)CC(CN(CC(O)CCCCCCCCCC)CC(O)CCCCCCCCCCCC)OCC)CC1)OCC]
(4-(3-((3-(4-(2-((3-(bis(2-hydroxytetradecyl)amino)-2-ethoxypropyl)(2-hydroxytetradecyl)amino)ethyl)piperazin-1-yl)-2-ethoxypropyl)(2-hydroxytetradecyl)amino)propanamido)-1-hydroxybutane-1,1-diyl)bis(phosphonic acid).

67. (canceled)
